(19) United States
(12) Patent Application Publication
(10) Pub. No.: US 2024/0277876 A1
(43) Pub. Date: Aug. 22, 2024

(54) PET IMAGING TRACERS

(71) Applicant: The Regents of the University of California, Oakland, CA (US)

(72) Inventor: Matthew F. L. Parker, San Francisco, CA (US)

(21) Appl. No.: 18/561,275

(22) PCT Filed: May 19, 2022
(86) PCT No.: PCT/US2022/030116

§ 371 Date: Nov. 15, 2023

Related U.S. Application Data
(60) Provisional application No. 63/191,259, filed on May 20, 2021.
(60) Provisional application No. 63/191,261, filed on May 20, 2021.
(60) Provisional application No. 63/191,262, filed on May 20, 2021.

(51) Int. Cl. A61K 51/04; A61K 51/08; A61K 51/10

(52) U.S. Cl. CPC A61K 51/047; A61K 51/0491; A61K 51/0497; A61K 51/083; A61K 51/088; A61K 51/1021; A61K 51/1051

(57) ABSTRACT

18-Fluoride is an ideal positron emitting radioisotope for imaging with peptides and peptide-like molecules due to the complementary biological half-like of peptides matched to the decay half-life of 18-fluoride (109 minutes). Disclosed herein, inter alia, are PET imaging tracer compounds and methods of use thereof.

### CROSS-REFERENCES TO RELATED APPLICATIONS

This application claims the benefit of U.S. Provisional Application No. 63/191,259, filed May 20, 2021; U.S. Provisional Application No. 63/191,261, filed May 20, 2021; U.S. Provisional Application No. 63/191,262, filed May 20, 2021, which are incorporated herein by reference in their entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-694001WO_Sequence_Listing_ST25.TXT, created May 13, 2022, 1,435 bytes, machine format IBM-PC, MS Windows operating system, is hereby incorporated by reference.

### BACKGROUND

18-Fluoride is an ideal positron emitting radioisotope for imaging with peptides and peptide-like molecules due to the complementary biological half-like of peptides matched to the decay half-life of 18-fluoride (109 minutes). Currently, nearly all tracers used in the clinic and in development suffer from isolation and chromatographic challenges. Disclosed herein, inter alia, are solutions to these and other problems in the art.

### BRIEF SUMMARY

In an aspect is provided a compound, or a pharmaceutically acceptable salt thereof, having the formula:

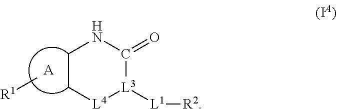

Ring A is a C₃-C₆ cycloalkylene, 3 to 6 membered heterocycloalkylene, C₅-C₆ arylene, or 5 to 6 membered heteroarylene.

L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L⁴ is a bond, —C(O)—, substituted or unsubstituted C₁-C₆ alkylene, or substituted or unsubstituted 2 to 6 membered heteroalkylene.

R¹ is a detectable moiety (e.g., radioisotope).

R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule.

R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl.

X² is —F, —Cl, —Br, or —I. The symbol n2 is an integer from 0 to 4. The symbols m2 and v2 are each independently 1 or 2.

In an aspect is provided a compound, or a pharmaceutically acceptable salt thereof, having the formula:

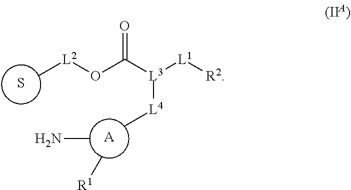

Ring A, L¹, L³, L⁴, R¹, and R² are as described herein, including in embodiments. S is a solid support.

L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a compound, or a pharmaceutically acceptable salt thereof, having the formula:

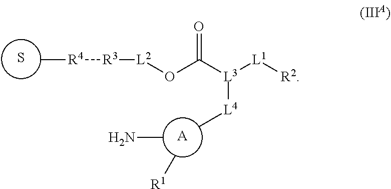

Ring A, S, L¹, L², L³, L⁴, R¹, and R² are as described herein, including in embodiments. R³ and R⁴ are an affinity ligand binding pair. The symbol ---- is a noncovalent or covalent bond.

In an aspect is provided a compound, or a pharmaceutically acceptable salt thereof, having the formula:

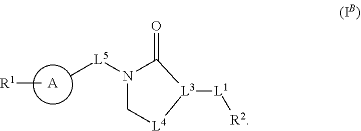

Ring A, L¹, L³, L⁴, R¹, and R² are as described herein, including in embodiments. L⁵ is a bond, —NH—, —O—, —S(O)₂—, —C(O)NH—, or substituted or unsubstituted C₁-C₆ alkylene.

In an aspect is provided a compound, or a pharmaceutically acceptable salt thereof, having the formula:

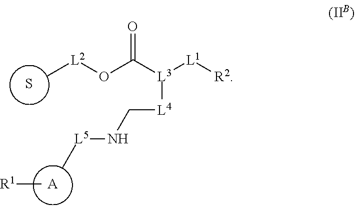

Ring A, L¹, L², L³, L⁴, L⁵, R¹, and R² are as described herein, including in embodiments. S is a solid support.

In an aspect is provided a compound, or a pharmaceutically acceptable salt thereof, having the formula:

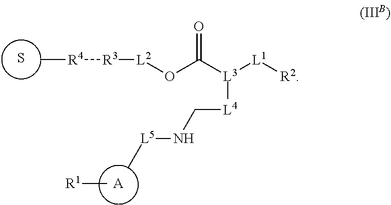

Ring A, S, L¹, L², L³, L⁴, L⁵, R¹, and R² are as described herein, including in embodiments. R³ and R⁴ are an affinity ligand binding pair. The symbol ---- is a noncovalent or covalent bond.

In an aspect is provided a compound, or a pharmaceutically acceptable salt thereof, having the formula:

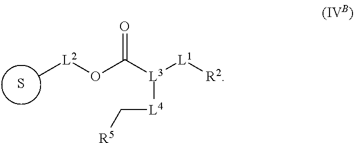

S, L¹, L², L³, L⁴, and R² are as described herein, including in embodiments. R⁵ is a leaving group.

In an aspect is provided a compound, or a pharmaceutically acceptable salt thereof, having the formula:

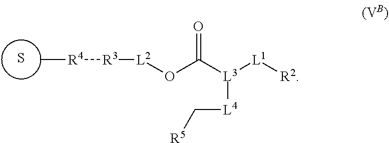

S, L¹, L², L³, L⁴, R², R³, R⁴, and R⁵ are as described herein, including in embodiments.

In an aspect is provided a compound, or a pharmaceutically acceptable salt thereof, having the formula:

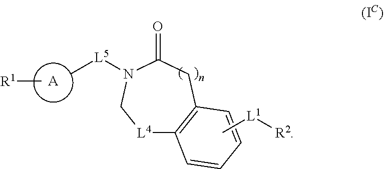

Ring A, L¹, L⁴, L⁵, R¹, and R² are as described herein, including in embodiments. The symbol n is 0 or 1.

In an aspect is provided a compound, or a pharmaceutically acceptable salt thereof, having the formula:

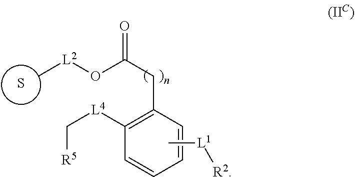

L¹, L², L⁴, R², and n are as described herein, including in embodiments. R⁵ is a leaving group. S is a solid support.

In an aspect is provided a compound, or a pharmaceutically acceptable salt thereof, having the formula:

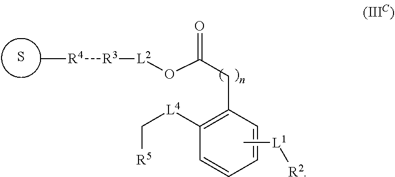

L¹, L², L⁴, R², R⁵, and n are as described herein, including in embodiments. R³ and R⁴ are an affinity ligand binding pair. The symbol ---- is a noncovalent or covalent bond.

In an aspect is provided a pharmaceutical composition including a compound described herein, or a pharmaceutically acceptable salt thereof, and a pharmaceutically acceptable excipient.

In an aspect is provided a method of detecting a level of a compound in a subject, the method including: (i) administering to the subject an effective amount of a compound described herein, or a pharmaceutically acceptable salt thereof, and (ii) detecting the level of the compound in the subject.

In an aspect is provided a method of detecting the level of CD44v6 in a subject, the method including administering to the subject an effective amount of a compound described herein, or a pharmaceutically acceptable salt thereof.

In an aspect is provided a method of making compound (I^{A}), or a pharmaceutically acceptable salt thereof, the method including mixing compound (II^{A}) or compound (III^{A}) in a reaction vessel; wherein compound (I^{A}) has the formula:

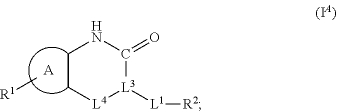

compound (II^{A}) has the formula:

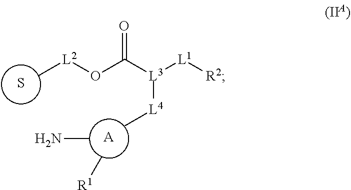

and compound (III^{A}) has the formula:

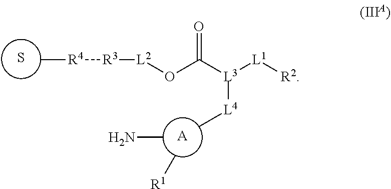

Ring A, S, L¹, L², L³, L⁴, R¹, R², R³, and R⁴ are as described herein, including in embodiments. The symbol ---- is a noncovalent or covalent bond.

In an aspect is provided a method of making compound (I^{B}), or a pharmaceutically acceptable salt thereof, the method including mixing compound (II^{B}) or compound (III^{B}) in a reaction vessel; wherein compound (I^{B}) has the formula:

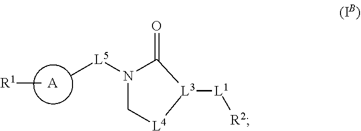

compound (II^{B}) has the formula:

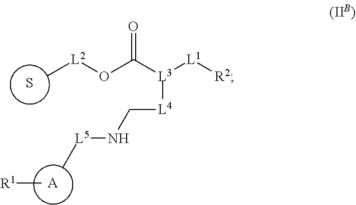

and compound (III^{B}) has the formula:

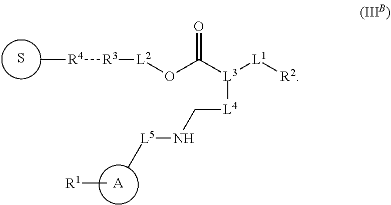

Ring A, S, L¹, L², L³, L⁴, R¹, R², R³, and R⁴ are as described herein, including in embodiments. The symbol ---- is a noncovalent or covalent bond.

In an aspect is provided a method of making compound (I^{B}), or a pharmaceutically acceptable salt thereof, the method including mixing compound (IV^{B}) or compound (V^{B}) and compound (A) in a reaction vessel; wherein compound (I^{B}) has the formula:

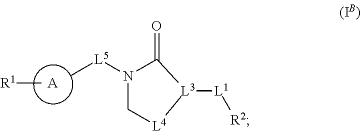

compound (IV^{B}) has the formula:

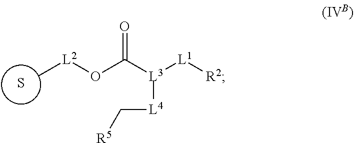

compound (V^{B}) has the formula:

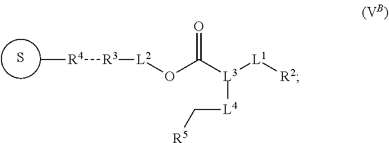

and compound (A) has the formula:

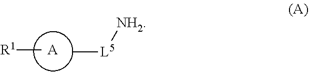

Ring A, S, L¹, L², L³, L⁴, L⁵, R¹, R², R³, R⁴, and R⁵ are as described herein, including in embodiments. The symbol ---- is a noncovalent or covalent bond.

In an aspect is provided a method of making compound (I^{C}), or a pharmaceutically acceptable salt thereof, the method including mixing compound (II^{C}) or compound (III^{C}) and compound (A) in a reaction vessel; wherein compound (I^{C}) has the formula:

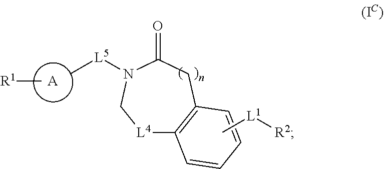

compound (II^{C}) has the formula:

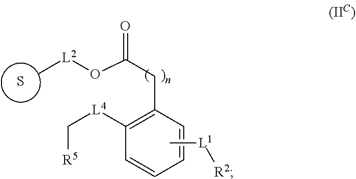

compound (III^{C}) has the formula:

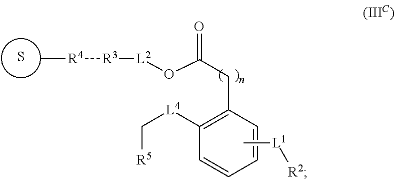

and compound (A) has the formula:

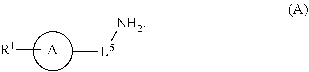

Ring A, S, L¹, L², L⁴, L⁵, R¹, R², R³, R⁴, R⁵, and n are as described herein, including in embodiments. The symbol ---- is a noncovalent or covalent bond.

### BRIEF DESCRIPTION OF THE DRAWINGS

FIG. 1. Overview of proposed assisted cleavage radiosynthesis of peptides from solid support (depicted as circles) with examples of linkers and labelled prosthetics.

FIG. 2. Quantification of the radio tracer library from solid support under pH 7.4 buffered saline and room temperature.

FIGS. 3A-3B. FIG. 3A: Schematic overview of the radiolabeling and release of sample peptide from solid support. The radiolabeled prosthetic (¹⁸F—Ar—C(O)—) is attached to the linker (proline). Acid treatment removes all protecting groups from the peptide and prosthetic. The radiolabeled peptide is liberated in pH 7.4 buffered saline at room temperature. Peptide sequence: GSGSGSGALAYADA (SEQ ID NO:1). FIG. 3B: HPLC analysis of the liberated peptide.

FIG. 4. Schematic overview of the prosthetic precursor synthesis and radiolabeling.

FIG. 5. Synthetic schemes of selected examples.

FIG. 6. Synthetic scheme of a solid supported GSGSGSGALAYADA (SEQ ID NO:1) peptide.

FIG. 7. Synthetic scheme of a solid supported DATFNWVFPVSVTFP (SEQ ID NO:2) peptide.

FIG. 8. Synthetic scheme of a solid supported RAGAYYVSSYRPGAW (SEQ ID NO:3) peptide.

FIG. 9. Synthetic scheme of a solid supported LPRDYAS (SEQ ID NO:4) peptide.

FIG. 10. Synthetic scheme of a solid supported DYGKNSW (SEQ ID NO:5) peptide.

FIG. 11. Synthetic scheme of a biotin GSGSGSGALAYADA (SEQ ID NO:1) peptide.

FIG. 12. Synthetic scheme of a dibenzoazacylooctyne (DBCO) GSGSGSGALAYADA (SEQ ID NO:1) peptide.

FIG. 13. Synthetic schemes of selected examples.

FIG. 14. Synthetic scheme of a solid supported GSGSGSGALAYADA (SEQ ID NO:1) peptide.

FIG. 15. Synthetic scheme of a solid supported DATFNWVFPVSVTFP (SEQ ID NO:2) peptide.

FIG. 16. Synthetic scheme of a solid supported RAGAYYVSSYRPGAW (SEQ ID NO:3) peptide.

FIG. 17. Synthetic scheme of a solid supported LPRDYAS (SEQ ID NO:4) peptide.

FIG. 18. Synthetic scheme of a solid supported DYGKNSW (SEQ ID NO:5) peptide.

FIG. 19. Synthetic scheme of a biotin GSGSGSGALAYADA (SEQ ID NO:1) peptide.

FIG. 20. Synthetic scheme of a DBCO GSGSGSGALAYADA (SEQ ID NO:1) peptide.

FIG. 21. Synthetic scheme of a solid supported GSGSGSGALAYADA (SEQ ID NO:1) peptide.

FIG. 22. Synthetic scheme of a solid supported DATFNWVFPVSVTFP (SEQ ID NO:2) peptide.

FIG. 23. Synthetic scheme of a solid supported RAGAYYVSSYRPGAW (SEQ ID NO:3) peptide.

FIG. 24. Synthetic scheme of a solid supported LPRDYAS (SEQ ID NO:4) peptide.

FIG. 25. Synthetic scheme of a solid supported DYGKNSW (SEQ ID NO:5) peptide.

FIG. 26. Synthetic scheme of a biotin GSGSGSGALAYADA (SEQ ID NO:1) peptide.

FIG. 27. Synthetic scheme of a DBCO GSGSGSGALAYADA (SEQ ID NO:1) peptide.

FIG. 28. Synthesis of a benzoyl hydrazide precursor and radiolabeling.

FIGS. 29A-29C. Labeling (FIG. 29A) and biological evaluation (FIGS. 29B-29C) of a prostate specific membrane antigen (PSMA) binding molecule.

FIGS. 30A-30C. Labeling (FIG. 30A) and biological evaluation (FIGS. 30B-30C) of an integrin receptor binding molecule.

FIGS. 31A-31C. Labeling (FIG. 31A) and biological evaluation (FIGS. 31B-31C) of a somatostatin receptor binding molecule.

FIGS. 32A-32C. Labeling (FIG. 32A) and biological evaluation (FIGS. 32B-32C) of a bacteria specific maltohexaose molecule.

FIGS. 33A-33C. Labeling (FIG. 33A) and biological evaluation (FIGS. 33B-33C) of a Her2 receptor binding nanobody.

FIGS. 34A-34C. Labeling (FIG. 34A) and biological evaluation (FIGS. 34B-34C) of a tumor necrosis factor alpha binding antibody fragment.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono-, or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). In embodiments, the heteroalkyl is fully saturated. In embodiments, the heteroalkyl is monounsaturated. In embodiments, the heteroalkyl is polyunsaturated. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkylene. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

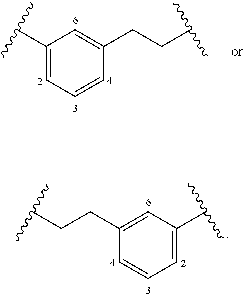 [SMILES: *CCc1cccc(*)c1 ; *CCc1cccc(*)c1]

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, —N₃, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), selenium (Se), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₅ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₅ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In some embodiments, the compound is a chemical species set forth in the application (e.g., Examples section, figures, or tables below).

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1} L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

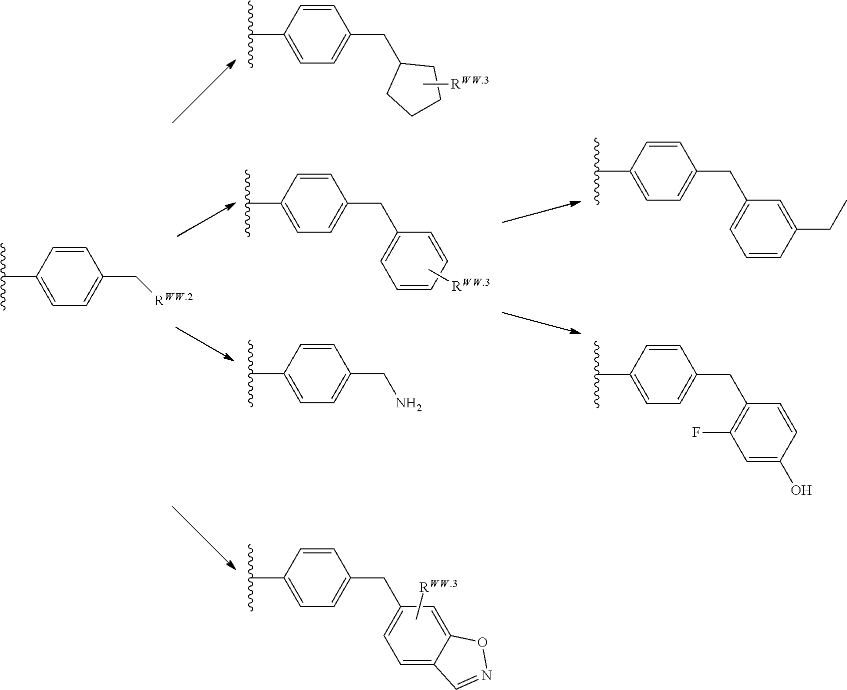

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100.B3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable agent” or “detectable moiety” is a substance, element, compound, or composition; or moiety thereof, detectable by appropriate means such as spectroscopic, photochemical, biochemical, immunochemical, chemical, magnetic resonance imaging, or other physical means. For example, useful detectable agents include ¹⁸F, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “biomolecule” is used in accordance with its plain and ordinary meaning and refers to a molecule found in nature or derivatives thereof, including macromolecules such as proteins, carbohydrates, lipids, and nucleic acids, as well as small molecules such as primary metabolites, secondary metabolites, and natural products. A biomolecule may be present as a moiety attached to the remainder of a compound. A biomolecule includes but is not limited to nucleic acids (e.g., DNA and RNA), peptide nucleic acids, sugars, peptides, proteins, antibodies, aptamers, lipids, and small molecule affinity ligands (e.g., inhibitors, biotin, and haptens).

The term “small molecule” is used in accordance with its well understood meaning and refers to a low molecular weight organic compound that may regulate a biological process. In embodiments, the small molecule is a compound that weighs less than 1000 daltons. In embodiments, the small molecule is a compound that weighs less than 900 daltons. In embodiments, the small molecule weighs less than 800 daltons. In embodiments, the small molecule weighs less than 700 daltons. In embodiments, the small molecule weighs less than 600 daltons. In embodiments, the small molecule weighs less than 500 daltons. In embodiments, the small molecule weighs less than 450 daltons. In embodiments, the small molecule weighs less than 400 daltons.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g., triflate), tosylates, mesylates, water, alcohols, nitrate, phosphate, thioether, amines, ammonia, fluoride, carboxylates, phenoxides, boronic acid, boronate esters, and alkoxides. In embodiments, the leaving group is designed to facilitate the reaction.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of PharmaceuticalScience, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL.sub.2), interferon alfa-2a; interferon alfa-2b; interferon alfa-n1; interferon alfa-n3; interferon beta-la; interferon gamma-lb; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol™ (i.e., paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP—XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DAIE), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The terms “polypeptide”, “peptide”, and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “antibody” is used in accordance with its plain and ordinary meaning and refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “nanobody” is used in accordance with its ordinary meaning and refers to an antibody fragment consisting of a single monomeric variable antibody domain. The term “Her2 receptor binding nanobody” as used herein refers to a nanobody capable of binding to the Her2 receptor.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments, contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist”, “activator”, “upregulator”, etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor”, “repressor”, “antagonist”, or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease means that the disease is caused by (in whole or in part), or a symptom of the disease is caused by (in whole or in part) the substance or substance activity or function.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propogated to other signaling pathway components. For example, binding of a thioredoxin protein with a compound as described herein may reduce the interactions between the thioredoxin protein and downstream effectors or signaling pathway components, resulting in changes in cell growth, proliferation, or survival.

In this disclosure, “comprises”, “comprising”, “containing”, and “having” and the like can have the meaning ascribed to them in U.S. patent law and can mean “includes”, “including”, and the like. “Consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with a compound, pharmaceutical composition, or method provided herein.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “cardiovascular disorder” or “cardiovascular disease” is used in accordance with its plain ordinary meaning. In embodiments, cardiovascular diseases that may be treated with a compound, pharmaceutical composition, or method described herein include, but are not limited to, stroke, heart failure, hypertension, hypertensive heart disease, myocardial infarction, angina pectoris, tachycardia, cardiomyopathy, rheumatic heart disease, cardiomyopathy, heart arrhythmia, congenital heart disease, valvular heart disease, carditis, aortic aneurysms, peripheral artery disease, thromboembolic disease, and venous thrombosis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. In embodiments, treating refers to treating a subject having a disease.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of a disease or disease symptoms in a patient. As indicated above, the prevention may be complete (no detectable symptoms) or partial, such that fewer symptoms are observed than would likely occur absent treatment.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” is meant that a composition described herein is administered at the same time, just prior to, or just after the administration of one or more additional therapies. The compounds provided herein can be administered alone or can be coadministered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

The term “CD44” refers to a glycoprotein involved in cell-cell interactions, cell adhesion, and migration. In embodiments, the CD44 protein is encoded by the CD44 gene. The term includes any recombinant or naturally-occurring form of CD44 or variants thereof that maintain CD44 activity (e.g., within at least 30%, 40%, 50%, 60%, 70%, 80%, 90%, 95%, or 100% activity compared to wildtype CD44). In embodiments, the CD44 protein has the amino acid sequence set forth in or corresponding to Entrez 960, UniProt P16070, RefSeq (protein) NP_000601.3, RefSeq (protein) NP_001001389.1, RefSeq (protein) NP_001001390.1, RefSeq (protein) NP_001001391.1, RefSeq (protein) NP_001001392.1, RefSeq (protein) NP_001189484.1, RefSeq (protein) NP_001189485.1, or RefSeq (protein) NP_001189456.1. In embodiments, the amino acid sequence or nucleic acid sequence is the sequence known at the time of filing of the present application.

The term “CD44v6” refers to an isoform of the family of CD44 glycoproteins.

The term “prostate specific membrane antigen” or “PSMA” refers to an enzyme that catalyzes the hydrolysis of N-acetylaspartylglutamate (NAAG) to glutamate and N-acetylaspartate (NAA). In embodiments, the PSMA protein is encoded by the FOLH1 gene. The term includes any recombinant or naturally-occurring form of PSMA or variants thereof that maintain PSMA activity (e.g., within at least 30%, 40%, 50%, 60%, 70%, 80%, 90%, 95%, or 100% activity compared to wildtype PSMA). In embodiments, the PSMA protein has the amino acid sequence set forth in or corresponding to Entrez 2346, UniProt Q04609, RefSeq (protein) NP_001014986.1, RefSeq (protein) NP_001180400.1, RefSeq (protein) NP_001180401.1, RefSeq (protein) NP_001180402.1, or RefSeq (protein) NP_004467.1. In embodiments, the amino acid sequence or nucleic acid sequence is the sequence known at the time of filing of the present application.

The term “integrin” is used in accordance with its ordinary meaning and refers to a transmembrane receptor that facilitates cell-cell and cell-extracellular matrix adhesion.

The term “somatostatin” refers to a protein involved in regulation of the endocrine system. In embodiments, the somatostatin protein is encoded by the SST gene. The term includes any recombinant or naturally-occurring form of somatostatin or variants thereof that maintain somatostatin activity (e.g., within at least 30%, 40%, 50%, 60%, 70%, 80%, 90%, 95%, or 100% activity compared to wildtype somatostatin). In embodiments, the somatostatin protein has the amino acid sequence set forth in or corresponding to Entrez 6750, UniProt P61278, or RefSeq (protein) NP_001039.1. In embodiments, the amino acid sequence or nucleic acid sequence is the sequence known at the time of filing of the present application.

The term “human epidermal growth factor receptor 2” or “Her2” or “HER2” refers to a protein involved in autophosphorylation of tyrosine residues. In embodiments, the Her2 protein is encoded by the ERBB2 gene. The term includes any recombinant or naturally-occurring form of Her2 or variants thereof that maintain Her2 activity (e.g., within at least 30%, 40%, 50%, 60%, 70%, 80%, 90%, 95%, or 100% activity compared to wildtype Her2). In embodiments, the Her2 protein has the amino acid sequence set forth in or corresponding to Entrez 2064, UniProt P04626, RefSeq (protein) NP_001005862.1, RefSeq (protein) NP_001276865.1, RefSeq (protein) NP_001276867.1, or RefSeq (protein) NP_004439.2. In embodiments, the amino acid sequence or nucleic acid sequence is the sequence known at the time of filing of the present application.

The term “tumor necrosis factor alpha” or “TNF-α” refers to a protein involved in cell signaling. In embodiments, the TNF-α protein is encoded by the TNF gene. The term includes any recombinant or naturally-occurring form of TNF-α or variants thereof that maintain TNF-α activity (e.g., within at least 30%, 40%, 50%, 60%, 70%, 80%, 90%, 95%, or 100% activity compared to wildtype TNF-α). In embodiments, the TNF-α protein has the amino acid sequence set forth in or corresponding to Entrez 7124, UniProt P01375, or RefSeq (protein) NP_000585.2. In embodiments, the amino acid sequence or nucleic acid sequence is the sequence known at the time of filing of the present application.

The term “positron emission tomography” or “PET” is used in accordance with its plain ordinary meaning and refers to an imaging technique that uses radioactive substances to visualize and measure metabolic processes. In embodiments, uses for PET scan include, but are not limited to, checking brain function; diagnosing cancer, heart problems, and brain disorders; examining blood flow to the heart; and determining spread of cancer and response to therapy.

### II. Compounds

In an aspect is provided a compound, or a pharmaceutically acceptable salt thereof, having the formula:

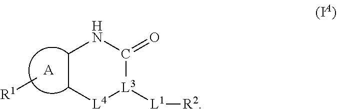

Ring A is a C₃-C₆ cycloalkylene, 3 to 6 membered heterocycloalkylene, phenylene, or 5 to 6 membered heteroarylene.

L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L³ is a substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L⁴ is a bond, —C(O)—, substituted or unsubstituted C₁-C₆ alkylene, or substituted or unsubstituted 2 to 6 membered heteroalkylene.

R¹ is a detectable moiety (e.g., radioisotope).

R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), or a monovalent form of a biomolecule.

R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered).

X² is —F, —Cl, —Br, or —I.

The symbol n2 is an integer from 0 to 4.

The symbols m2 and v2 are each independently 1 or 2.

In embodiments, the compound, or a pharmaceutically acceptable salt thereof, has the formula:

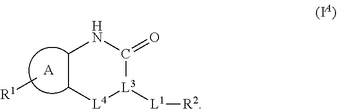

Ring A is a C₃-C₆ cycloalkylene, 3 to 6 membered heterocycloalkylene, phenylene, or 5 to 6 membered heteroarylene. L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR⁰—, —NR¹⁰C(NH)NR¹⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). L³ is a substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). L⁴ is —C(O)—, substituted or unsubstituted C₁-C₆ alkylene, or substituted or unsubstituted 2 to 6 membered heteroalkylene. R¹ is a detectable moiety (e.g., radioisotope). R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered). X² is —F, —Cl, —Br, or —I. The symbol n2 is an integer from 0 to 4. The symbols m2 and v2 are each independently 1 or 2.

In an aspect is provided a compound, or a pharmaceutically acceptable salt thereof, having the formula:

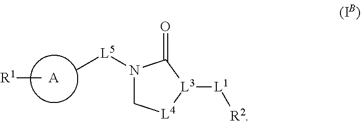

Ring A is a C₃-C₆ cycloalkylene, 3 to 6 membered heterocycloalkylene, phenylene, or 5 to 6 membered heteroarylene.

L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L³ is a substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L⁴ is a bond, —C(O)—, substituted or unsubstituted C₁-C₆ alkylene, or substituted or unsubstituted 2 to 6 membered heteroalkylene.

L⁵ is a bond, —NH—, —O—, —S(O)₂—, —C(O)NH—, or substituted or unsubstituted C₁-C₆ alkylene.

R¹ is a detectable moiety (e.g., radioisotope).

R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), or a monovalent form of a biomolecule.

R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered).

X² is —F, —Cl, —Br, or —I.

The symbol n2 is an integer from 0 to 4.

The symbols m2 and v2 are each independently 1 or 2.

In embodiments, the compound, or a pharmaceutically acceptable salt thereof, has the formula:

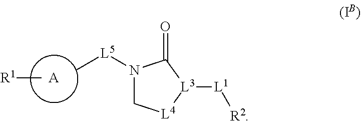

Ring A is a C₃-C₆ cycloalkylene, 3 to 6 membered heterocycloalkylene, phenylene, or 5 to 6 membered heteroarylene. L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). L³ is a substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). L⁴ is —C(O)—, substituted or unsubstituted C₁-C₆ alkylene, or substituted or unsubstituted 2 to 6 membered heteroalkylene. L⁵ is a bond, —NH—, —O—, —S(O)₂—, —C(O)NH—, or substituted or unsubstituted C₁-C₆ alkylene. R¹ is a detectable moiety (e.g., radioisotope). R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered). X² is —F, —Cl, —Br, or —I. The symbol n2 is an integer from 0 to 4. The symbols m2 and v2 are each independently 1 or 2.

In an aspect is provided a compound, or a pharmaceutically acceptable salt thereof, having the formula:

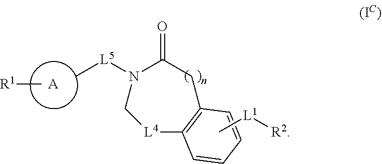

Ring A is a C₃-C₆ cycloalkylene, 3 to 6 membered heterocycloalkylene, phenylene, or 5 to 6 membered heteroarylene.

L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L⁴ is a bond, —C(O)—, substituted or unsubstituted C₁-C₆ alkylene, or substituted or unsubstituted 2 to 6 membered heteroalkylene.

L⁵ is a bond, —NH—, —O—, —S(O)₂—, —C(O)NH—, or substituted or unsubstituted C₁-C₆ alkylene.

R¹ is a detectable moiety (e.g., radioisotope).

R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), or a monovalent form of a biomolecule.

R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered).

X² is —F, —Cl, —Br, or —I.

The symbol n2 is an integer from 0 to 4.

The symbols m2 and v2 are each independently 1 or 2.

The symbol n is 0 or 1.

In embodiments, the compound, or a pharmaceutically acceptable salt thereof, has the formula:

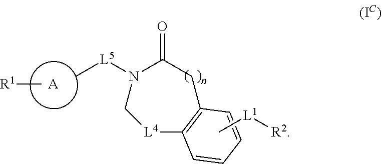

Ring A is a C₃-C₆ cycloalkylene, 3 to 6 membered heterocycloalkylene, phenylene, or 5 to 6 membered heteroarylene. L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR⁰—, —NR¹⁰C(NH)NR¹⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). L⁴ is a bond, —C(O)—, substituted or unsubstituted C₁-C₆ alkylene, or substituted or unsubstituted 2 to 6 membered heteroalkylene. L⁵ is a bond, —NH—, —O—, —S(O)₂—, —C(O)NH—, or substituted or unsubstituted C₁-C₆ alkylene. R¹ is a detectable moiety (e.g., radioisotope). R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered). X² is —F, —Cl, —Br, or —I. The symbol n2 is an integer from 0 to 4. The symbols m2 and v2 are each independently 1 or 2. The symbol n is 0 or 1.

In embodiments, Ring A is a C₃-C₆ cycloalkylene, 3 to 6 membered heterocycloalkylene, or phenylene. In embodiments, Ring A is a C₃-C₆ cycloalkylene. In embodiments, Ring A is a 3 to 6 membered heterocycloalkylene. In embodiments, Ring A is phenylene. In embodiments, Ring A is a 5 to 6 membered heteroarylene.

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a substituted or unsubstituted heteroalkylene or substituted or unsubstituted heterocycloalkylene. In embodiments, L³ is an unsubstituted heteroalkylene. In embodiments, L³ is an unsubstituted heterocycloalkylene. In embodiments, L³ is a substituted or unsubstituted 2 to 4 membered heteroalkylene or substituted or unsubstituted 3 to 6 membered heterocycloalkylene. In embodiments, L³ is an unsubstituted 2 membered heteroalkylene. In embodiments, L³ is an unsubstituted 3 membered heteroalkylene. In embodiments, L³ is an unsubstituted 4 membered heteroalkylene. In embodiments, L³ is an unsubstituted 3 membered heterocycloalkylene. In embodiments, L³ is an unsubstituted 4 membered heterocycloalkylene. In embodiments, L³ is an unsubstituted 5 membered heterocycloalkylene. In embodiments, L³ is an unsubstituted 6 membered heterocycloalkylene.

In embodiments, the compound has the formula:

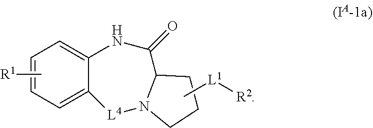

L¹, L⁴, R¹, and R² are as described herein, including in embodiments.

In embodiments, the compound has the formula:

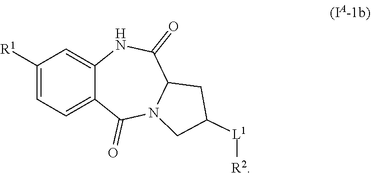

L¹, R¹ and R² are as described herein, including in embodiments.

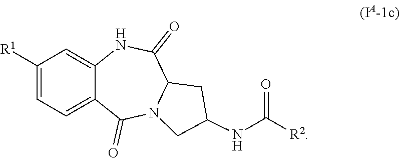

In embodiments, the compound has the formula:

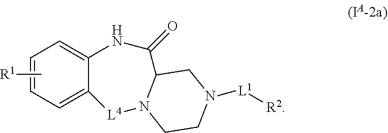

L¹, L⁴, R¹, and R² are as described herein, including in embodiments.

In embodiments, the compound has the formula:

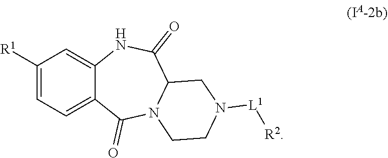

L¹, R¹, and R² are as described herein, including in embodiments.

In embodiments, the compound has the formula:

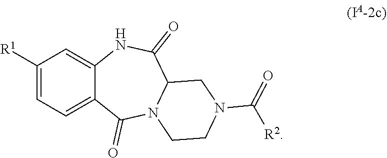

R¹ and R² are as described herein, including in embodiments.

In embodiments, the compound has the formula:

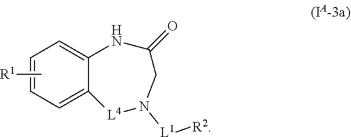

L¹, L⁴, R¹, and R² are as described herein, including in embodiments.

In embodiments, the compound has the formula:

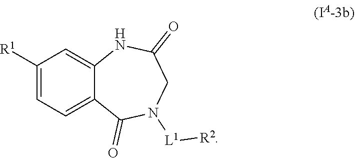

L¹, R¹, and R² are as described herein, including in embodiments.

In embodiments, the compound has the formula:

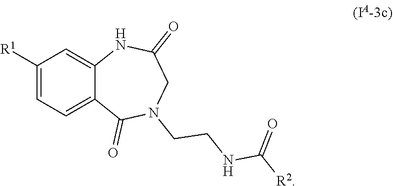

R¹ and R² are as described herein, including in embodiments.

In embodiments, the compound has the formula:

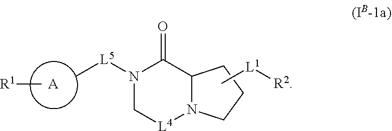

Ring A, L¹, L⁴, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

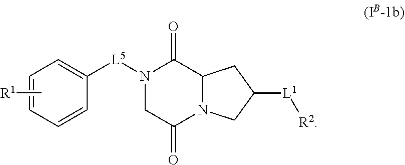

L¹, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

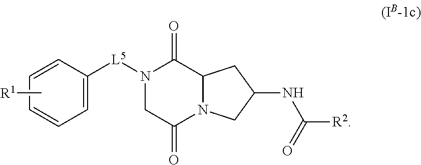

L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

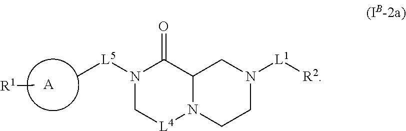

Ring A, L¹, L⁴, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

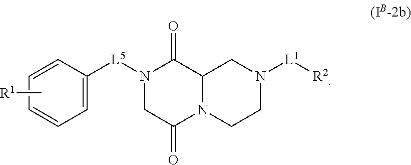

L¹, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

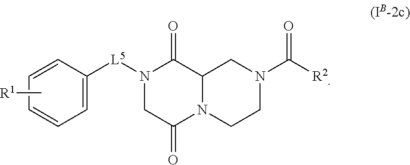

L⁵, R¹, and R² are as described herein, including in embodiments. In embodiments, L is a bond. In embodiments, L⁵ is —NH—. In embodiments, L⁵ is —O—. In embodiments, L⁵ is —C(O)NH—.

In embodiments, the compound has the formula:

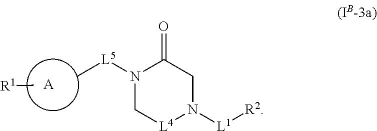

Ring A, L¹, L⁴, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

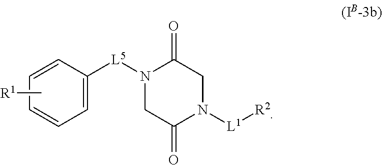

L¹, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

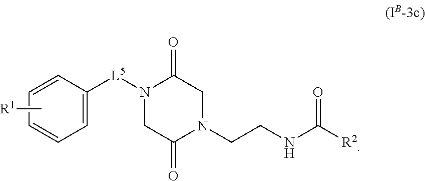

L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

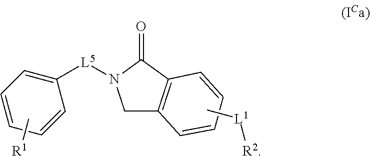

L¹, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

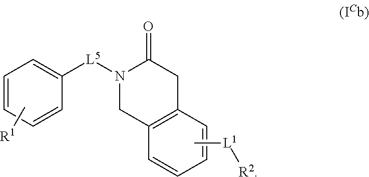

L¹, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

In embodiments, L¹ is —NHC(O)—. In embodiments, L¹ is —C(O)—. In embodiments, L¹ is a substituted or unsubstituted heteroalkylene. In embodiments, L¹ is a substituted or unsubstituted 2 to 10 membered heteroalkylene. In embodiments, L¹ is a substituted or unsubstituted 2 to 6 membered heteroalkylene. In embodiments, L¹ is a substituted 2 to 6 membered heteroalkylene. In embodiments, L¹ is -(unsubstituted C₁-C₄ alkylene)-NHC(O)—. In embodiments, L¹ is —CH₂CH₂NHC(O)—. In embodiments, L¹ is —NHC(O)-(unsubstituted C₁-C₄ alkylene)-C(O)—. In embodiments, L¹ is —NHC(O)—(CH₂)—NH. In embodiments, L¹ is —NHC(O)—(CH₂)₂—NH. In embodiments, L¹ is —NHC(O)—(CH₂)₃—NH. In embodiments, L¹ is —NHC(O)—(CH₂)₄—NH.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is independently hydrogen or substituted or unsubstituted alkyl. In embodiments, R¹⁰ is independently hydrogen or substituted or unsubstituted C₁-C₄ alkyl. In embodiments, R¹⁰ is independently hydrogen. In embodiments, R¹⁰ is independently unsubstituted C₁-C₄ alkyl. In embodiments, R¹⁰ is independently unsubstituted methyl. In embodiments, R¹⁰ is independently unsubstituted ethyl. In embodiments, R¹⁰ is independently unsubstituted propyl. In embodiments, R¹⁰ is independently unsubstituted n-propyl. In embodiments, R¹⁰ is independently unsubstituted isopropyl. In embodiments, R¹⁰ is independently unsubstituted butyl. In embodiments, R¹⁰ is independently unsubstituted n-butyl. In embodiments, R¹⁰ is independently unsubstituted tert-butyl.

In embodiments, a substituted L⁴ (e.g., substituted alkylene and/or substituted heteroalkylene) is substituted with at least one substituent group, size-limited substituent group, or lower substituent group; wherein if the substituted L⁴ is substituted with a plurality of groups selected from substituent groups, size-limited substituent groups, and lower substituent groups; each substituent group, size-limited substituent group, and/or lower substituent group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, L⁴ is —C(O)—, substituted or unsubstituted C₁-C₆ alkylene, or substituted or unsubstituted 2 to 6 membered heteroalkylene. In embodiments, L⁴ is a bond, —C(O)—, or unsubstituted methylene. In embodiments, L⁴ is —C(O)— or unsubstituted methylene. In embodiments, L⁴ is a bond. In embodiments, L⁴ is —C(O)—. In embodiments, L⁴ is unsubstituted methylene.

In embodiments, a substituted L⁵ (e.g., substituted alkylene) is substituted with at least one substituent group, size-limited substituent group, or lower substituent group; wherein if the substituted L⁵ is substituted with a plurality of groups selected from substituent groups, size-limited substituent groups, and lower substituent groups; each substituent group, size-limited substituent group, and/or lower substituent group may optionally be different. In embodiments, when L⁵ is substituted, it is substituted with at least one substituent group. In embodiments, when L⁵ is substituted, it is substituted with at least one size-limited substituent group. In embodiments, when L⁵ is substituted, it is substituted with at least one lower substituent group.

In embodiments, L⁵ is a bond, —NH—, —O—, or —C(O)NH—. In embodiments, L⁵ is a bond. In embodiments, L⁵ is —NH—. In embodiments, L⁵ is —O—. In embodiments, L⁵ is —S(O)₂—. In embodiments, L⁵ is —C(O)NH—. In embodiments, L⁵ is —NHC(O)—. In embodiments, L⁵ is substituted or unsubstituted C₁-C₆ alkylene.

In embodiments, n is 0. In embodiments, n is 1.

In embodiments, R¹ is a radioisotope. In embodiments, R¹ is a PET detectable radioisotope. In embodiments, R¹ is —¹⁸F. In embodiments, R¹ is —⁷⁶Br. In embodiments, R¹ is —⁷⁷Br. In embodiments, R¹ is —¹²³I. In embodiments, R¹ is —¹²⁴I. In embodiments, R¹ is —¹²⁵I. In embodiments, R¹ is —¹³¹I.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is a monovalent form of a drug or substituted heteroalkyl. In embodiments, R² is a monovalent form of a drug. In embodiments, R² is a substituted heteroalkyl. In embodiments, R² is a substituted 2 to 100 membered heteroalkyl. In embodiments, R² is a substituted 2 to 80 membered heteroalkyl. In embodiments, R² is a substituted 2 to 60 membered heteroalkyl. In embodiments, R² is a substituted 2 to 40 membered heteroalkyl. In embodiments, R² is a substituted 2 to 20 membered heteroalkyl. In embodiments, R² is a monovalent form of a peptide. In embodiments, R² is a monovalent form of a peptide having the sequence GSGSGSGALAYADA (SEQ ID NO:1). In embodiments, R² is a monovalent form of a peptide having the sequence DATFNWVFPVSVTFP (SEQ ID NO:2). In embodiments, R² is a monovalent form of a peptide having the sequence RAGAYYVSSYRPGAW (SEQ ID NO:3). In embodiments, R² is a monovalent form of a peptide having the sequence LPRDYAS (SEQ ID NO:4). In embodiments, R² is a monovalent form of a peptide having the sequence DYGKNSW (SEQ ID NO:5).

In embodiments, R² is a monovalent form of a biomolecule.

In embodiments, R² is a monovalent form of a prostate specific membrane antigen (PSMA) binding molecule. In embodiments, R² is a monovalent form of a compound as described in Carlucci, G. et al., J. Nucl. Med. 2021, 62, 149-155, which is herein incorporated by reference in its entirety for all purposes. In embodiments, R² is a monovalent form of a compound as described in Banerjee, S. R. et al., Bioconjugate Chem. 2016, 27, 1447-1455, which is herein incorporated by reference in its entirety for all purposes. In embodiments, R² is

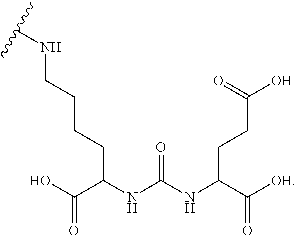 [SMILES: *NCCCCC(NC(=O)NC(CCC(=O)O)C(=O)O)C(=O)O]

In embodiments, R² is

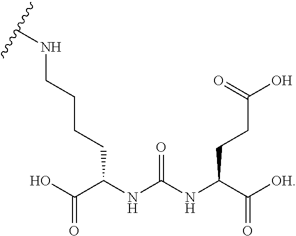 [SMILES: *NCCCC[C@H](NC(=O)N[C@@H](CCC(=O)O)C(=O)O)C(=O)O]

In embodiments, R² is a monovalent form of an integrin receptor binding molecule. In embodiments, R² is a monovalent form of a compound as described in Dumont, R. A. et al., J. Nucl. Med. 2011, 52, 1276-1284, which is herein incorporated by reference in its entirety for all purposes. In embodiments, R² is

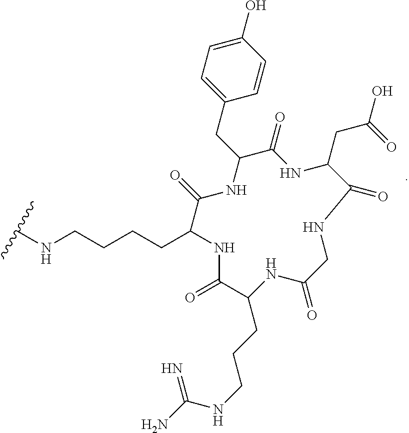 [SMILES: *NCCCCC1NC(=O)C(CCCNC(=N)N)NC(=O)CNC(=O)C(CC(=O)O)NC(=O)C(Cc2ccc(O)cc2)NC1=O]

In embodiments, R² is

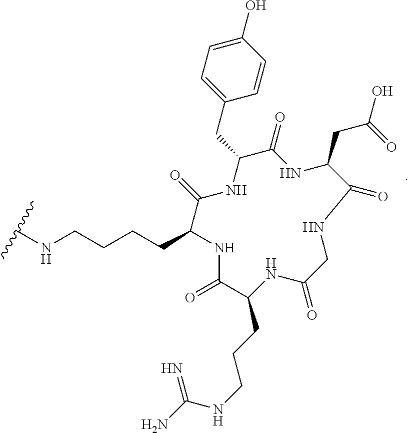 [SMILES: *NCCCC[C@@H]1NC(=O)[C@H](CCCNC(=N)N)NC(=O)CNC(=O)[C@H](CC(=O)O)NC(=O)[C@@H](Cc2ccc(O)cc2)NC1=O]

In embodiments, R² is a monovalent form of a somatostatin binding molecule. In embodiments, R² is a monovalent form of a compound as described in Hennrich, U. et al., Pharmaceuticals 2020, 13, 38, which is herein incorporated by reference in its entirety for all purposes. In embodiments, R² is

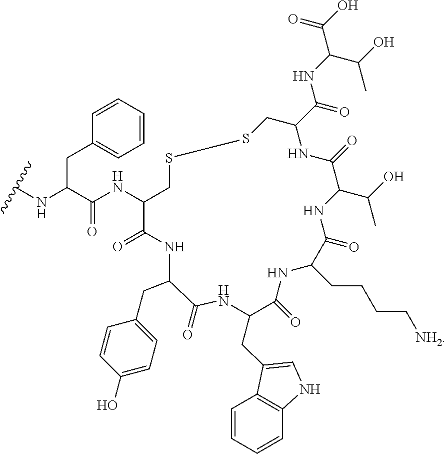 [SMILES: *NC(Cc1ccccc1)C(=O)NC1CSSCC(C(=O)NC(C(=O)O)C(C)O)NC(=O)C(C(C)O)NC(=O)C(CCCCN)NC(=O)C(Cc2c[nH]c3ccccc23)NC(=O)C(Cc2ccc(O)cc2)NC1=O]

In embodiments, R² is

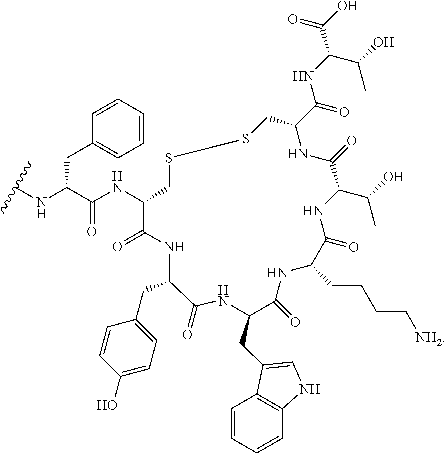 [SMILES: *N[C@H](Cc1ccccc1)C(=O)N[C@@H]1CSSC[C@H](C(=O)N[C@H](C(=O)O)[C@@H](C)O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O]

In embodiments, R² is a monovalent form of a bacteria specific maltohexaose molecule. In embodiments, R² is a monovalent form of a compound as described in Ning, X. et al., Angew. Chem. Int. Ed. 2014, 53, 14096-14101, which is herein incorporated by reference in its entirety for all purposes. In embodiments, R² is

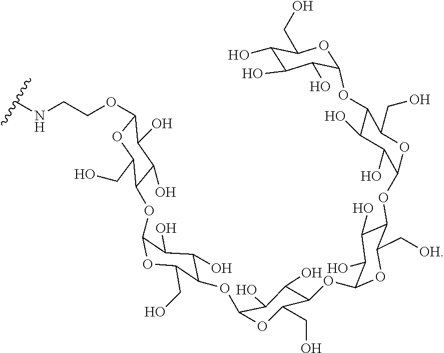 [SMILES: *NCCOC1OC(CO)C(OC2OC(CO)C(OC3OC(CO)C(OC4OC(CO)C(OC5OC(CO)C(OC6OC(CO)C(O)C(O)C6O)C(O)C5O)C(O)C4O)C(O)C3O)C(O)C2O)C(O)C1O]

In embodiments, R² is a monovalent form of a Her2 receptor binding molecule. In embodiments, R² is a monovalent form of a Her2 receptor binding nanobody. In embodiments, R² is a monovalent form of a compound as described in Vaidyanathan, G. et al., J. Nucl. Med. 2016, 57, 967-973, which is herein incorporated by reference in its entirety for all purposes. In embodiments, R² is

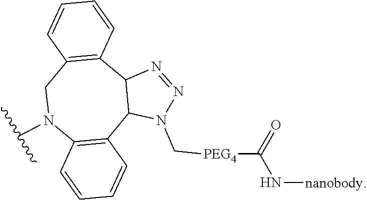

In embodiments, R² is

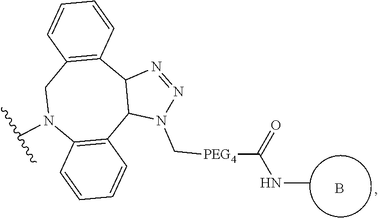

wherein B is a monovalent form of a Her2 receptor binding nanobody.

In embodiments, R² is a monovalent form of a TNF-α binding antibody molecule. In embodiments, R² is a monovalent form of a compound as described in Lee, J. U. et al., Int. J. Mol. Sci. 2017, 18, 228, which is herein incorporated by reference in its entirety for all purposes. In embodiments, R² is a monovalent form of infliximab. In embodiments, R² is a monovalent form of adalimumab. In embodiments, R² is a monovalent form of golimumab. In embodiments, R² is a monovalent form of certolizumab pegol. In embodiments, R² is

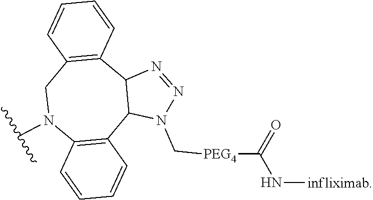

In embodiments, R² is

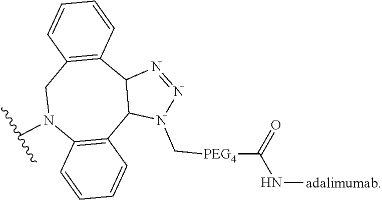

In embodiments, R² is

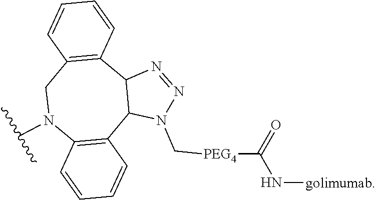

In embodiments, R² is

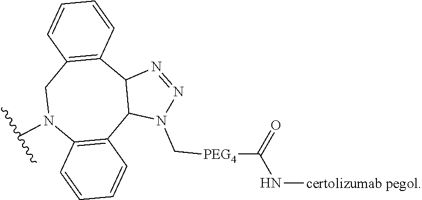

In embodiments, R² is

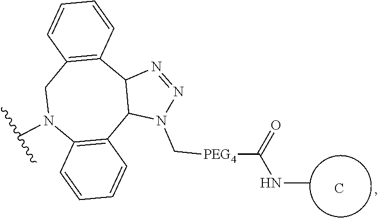

wherein is a monovalent form of a TNF-α binding antibody.

In an aspect is provided a compound, or a pharmaceutically acceptable salt thereof, having the formula:

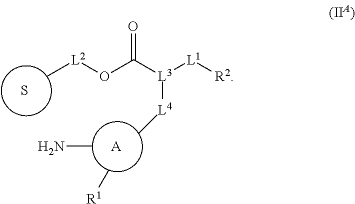

Ring A, L¹, L³, L⁴, R¹, and R² are as described herein, including in embodiments.

L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

S is a solid support.

In embodiments, the compound has the formula:

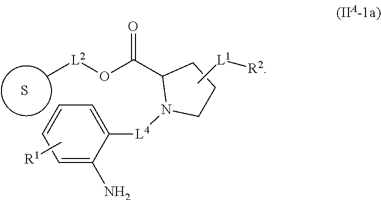

S, L¹, L², L⁴, R¹, and R² are as described herein, including in embodiments.

In embodiments, the compound has the formula:

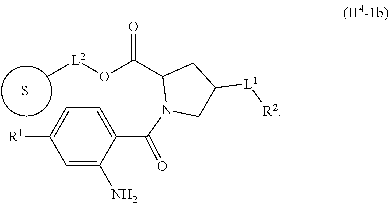

S, L¹, L², R¹, and R² are as described herein, including in embodiments.

In embodiments, the compound has the formula:

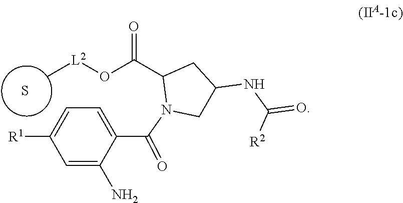

S, L², R¹, and R² are as described herein, including in embodiments.

In embodiments, the compound has the formula:

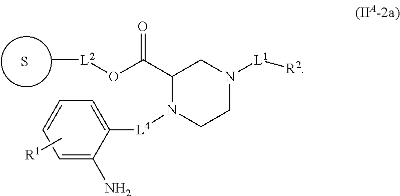

S, L¹, L², L⁴, R¹, and R² are as described herein, including in embodiments.

In embodiments, the compound has the formula:

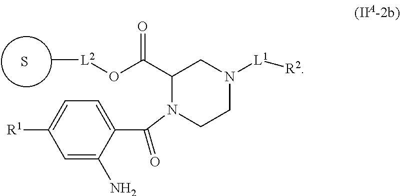

S, L¹, L², R¹, and R² are as described herein, including in embodiments.

In embodiments, the compound has the formula:

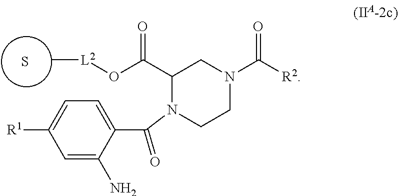

S, L², R¹, and R² are as described herein, including in embodiments.

In embodiments, the compound has the formula:

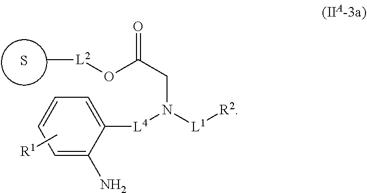

S, L¹, L², L⁴, R¹, and R² are as described herein, including in embodiments.

In embodiments, the compound has the formula:

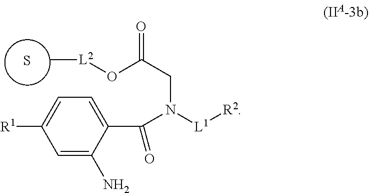

S, L¹, L², R¹, and R² are as described herein, including in embodiments.

In embodiments, the compound has the formula:

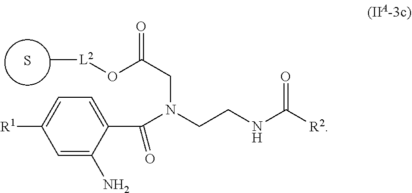

S, L², R¹, and R² are as described herein, including in embodiments.

In an aspect is provided a compound, or a pharmaceutically acceptable salt thereof, having the formula:

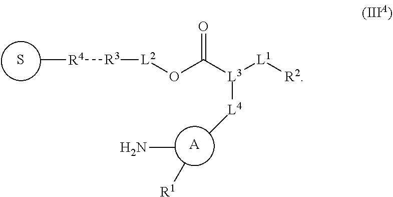

Ring A, S, L¹, L², L³, L⁴, R¹, and R² are as described herein, including in embodiments. R³ and R⁴ are an affinity ligand binding pair. The symbol ---- is a noncovalent or covalent bond.

In embodiments, the compound has the formula:

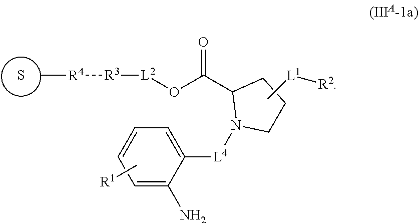

S, L¹, L², L⁴, R¹, R², R³, and R⁴ are as described herein, including in embodiments.

In embodiments, the compound has the formula:

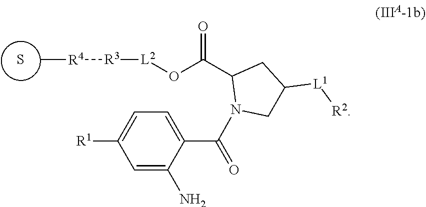

S, L¹, L², R¹, R², R³, and R⁴ are as described herein, including in embodiments.

In embodiments, the compound has the formula:

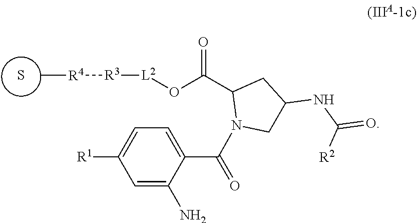

S, L², R¹, R², R³, and R⁴ are as described herein, including in embodiments.

In embodiments, the compound has the formula:

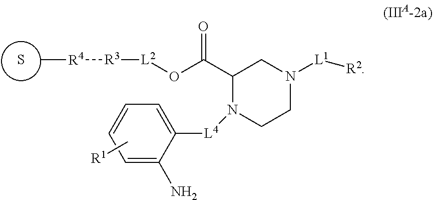

S, L¹, L², L⁴, R¹, R², R³, and R⁴ are as described herein, including in embodiments.

In embodiments, the compound has the formula:

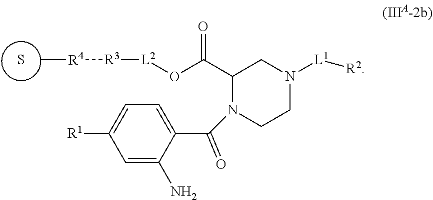

S, L¹, L², R¹, R², R³, and R⁴ are as described herein, including in embodiments.

In embodiments, the compound has the formula:

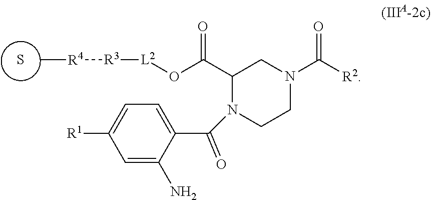

S, L², R¹, R², R³, and R⁴ are as described herein, including in embodiments.

In embodiments, the compound has the formula:

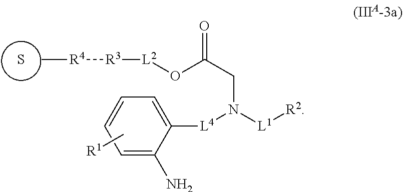

S, L¹, L², L⁴, R¹, R², R³, and R⁴ are as described herein, including in embodiments.

In embodiments, the compound has the formula:

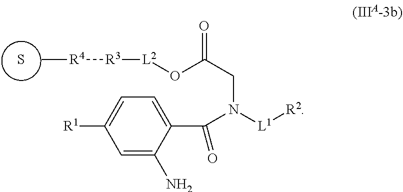

S, L¹, L², R¹, R², R³, and R⁴ are as described herein, including in embodiments.

In embodiments, the compound has the formula:

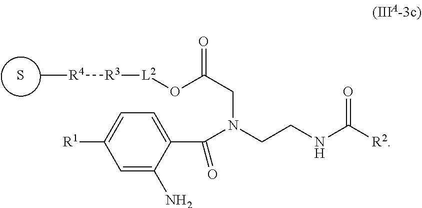

S, L², R¹, R², R³, and R⁴ are as described herein, including in embodiments.

In an aspect is provided a compound, or a pharmaceutically acceptable salt thereof, having the formula:

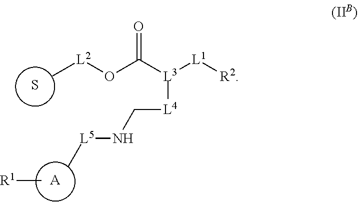

Ring A, L¹, L³, L⁴, L⁵, R¹, and R² are as described herein, including in embodiments.

L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

S is a solid support.

In embodiments, the compound has the formula:

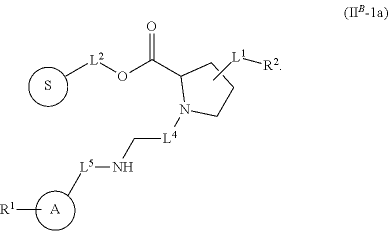

Ring A, S, L¹, L², L⁴, L⁵, R¹, and R² are as described herein, including in embodiments.

In embodiments, the compound has the formula:

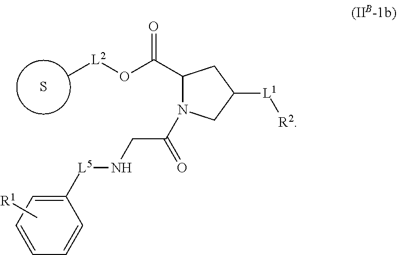

S, L¹, L², L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

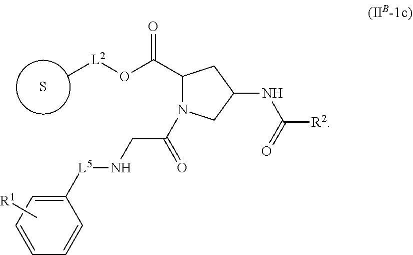

S, L², L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

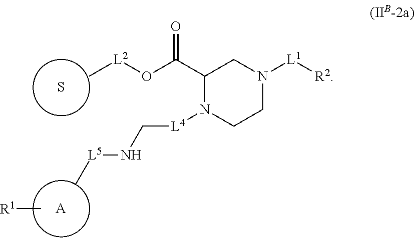

Ring A, S, L¹, L², L⁴, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

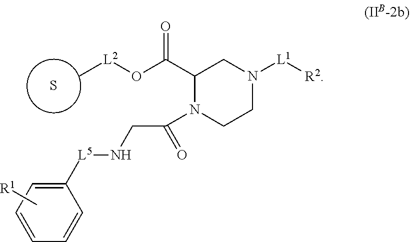

S, L¹, L², L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

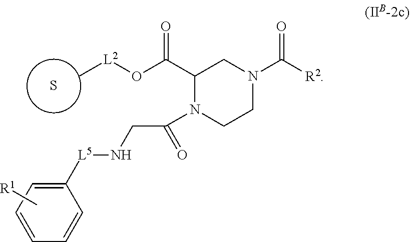

S, L², L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

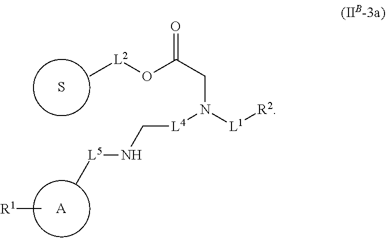

Ring A, S, L¹, L², L⁴, L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

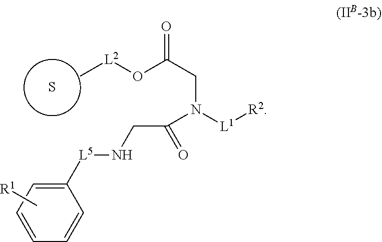

S, L¹, L², L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

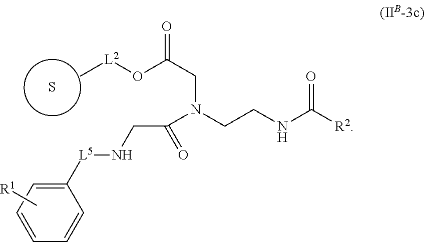

S, L², L⁵, R¹, and R² are as described herein, including in embodiments. In embodiments, L⁵ is a bond. In embodiments, L⁵ is —NH—. In embodiments, L⁵ is —O—. In embodiments, L⁵ is —C(O)NH—.

In an aspect is provided a compound, or a pharmaceutically acceptable salt thereof, having the formula:

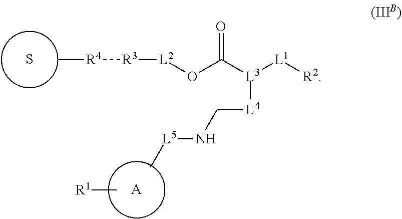

Ring A, S, L¹, L², L³, L⁴, L⁵, R¹, and R² are as described herein, including in embodiments. R³ and R⁴ are an affinity ligand binding pair. The symbol ---- is a noncovalent or covalent bond.

In embodiments, the compound has the formula:

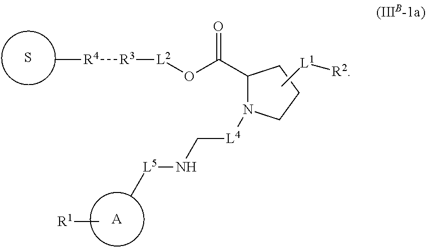

Ring A, S, L¹, L², L⁴, L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

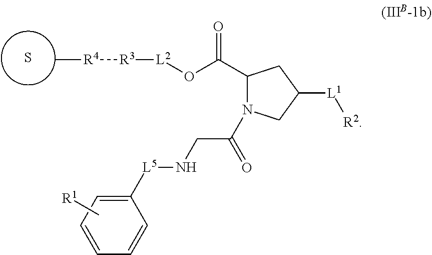

S, L¹, L², L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

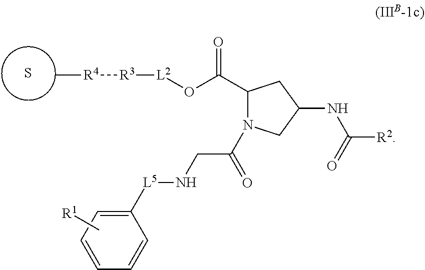

S, L², L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

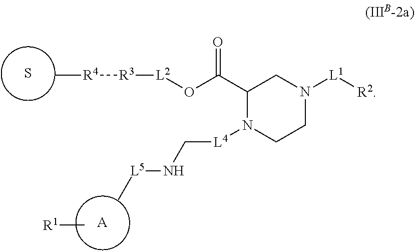

Ring A, S, L¹, L², L⁴, L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

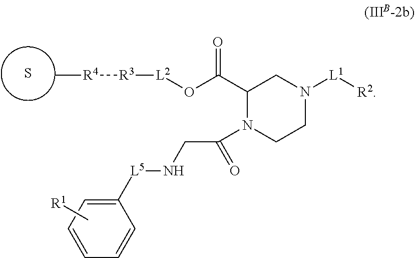

S, L¹, L², L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

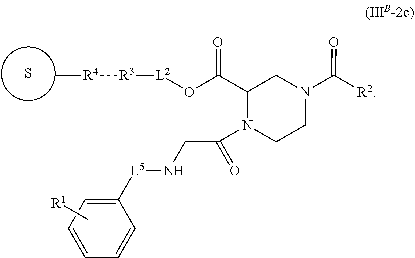

S, L², L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

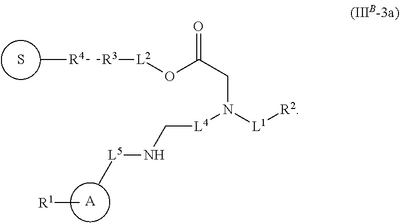

Ring A, S, L¹, L², L⁴, L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

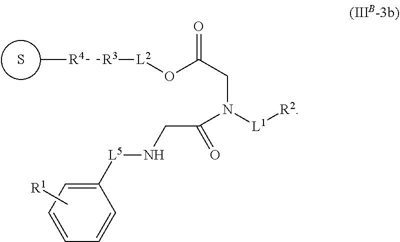

S, L¹, L², L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In embodiments, the compound has the formula:

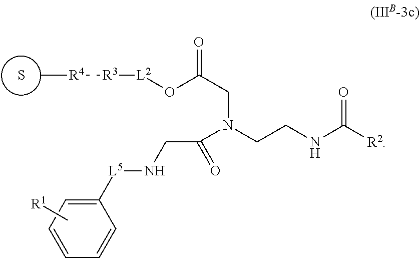

S, L², L⁵, R¹, R², R³, and R⁴ are as described herein, including in embodiments. In embodiments, L⁵ is a bond. In embodiments, L⁵ is —NH—. In embodiments, L⁵ is —O—. In embodiments, L⁵ is —C(O)NH—.

In an aspect is provided a compound, or a pharmaceutically acceptable salt thereof, having the formula:

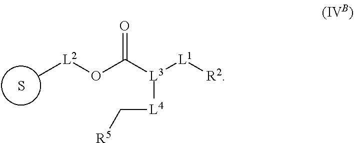

S, L¹, L², L³, L⁴, and R² are as described herein, including in embodiments. R⁵ is a leaving group.

In embodiments, the compound has the formula:

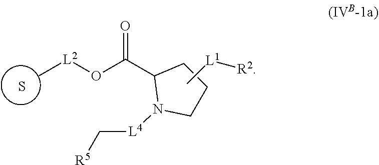

S, L¹, L², L⁴, R², and R⁵ are as described herein, including in embodiments.

In embodiments, the compound has the formula:

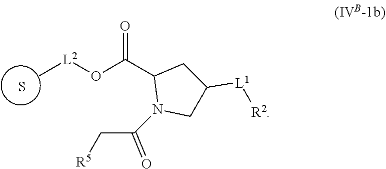

S, L¹, L², R², and R⁵ are as described herein, including in embodiments.

In embodiments, the compound has the formula:

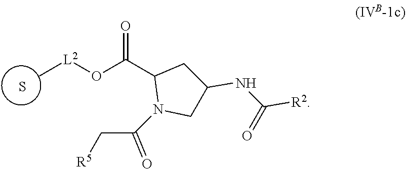

S, L², R², and R⁵ are as described herein, including in embodiments.

In embodiments, the compound has the formula:

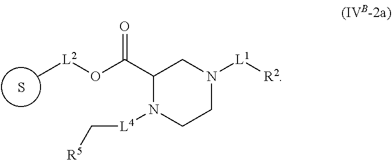

S, L¹, L², L⁴, R², and R⁵ are as described herein, including in embodiments.

In embodiments, the compound has the formula:

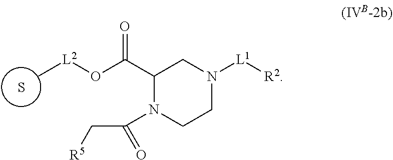

S, L¹, L², R², and R⁵ are as described herein, including in embodiments.

In embodiments, the compound has the formula:

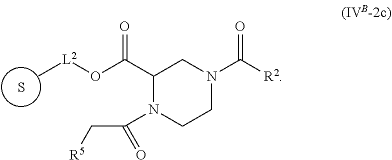

S, L², R², and R⁵ are as described herein, including in embodiments.

In embodiments, the compound has the formula:

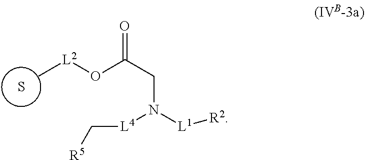

S, L¹, L², L⁴, R², and R⁵ are as described herein, including in embodiments.

In embodiments, the compound has the formula:

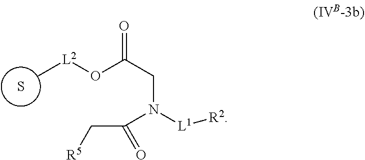

S, L¹, L², R², and R⁵ are as described herein, including in embodiments.

In embodiments, the compound has the formula:

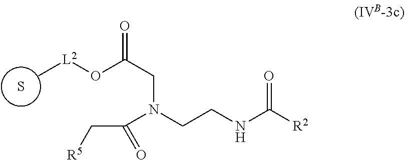

S, L², R², and R⁵ are as described herein, including in embodiments.

In an aspect is provided a compound, or a pharmaceutically acceptable salt thereof, having the formula:

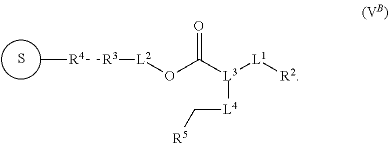

S, L¹, L², L³, L⁴, R², R³, R⁴, and R⁵ are as described herein, including in embodiments.

In embodiments, the compound has the formula:

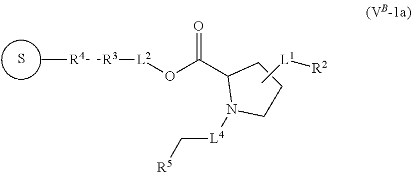

S, L¹, L², L⁴, R², R³, R⁴, and R⁵ are as described herein, including in embodiments.

In embodiments, the compound has the formula:

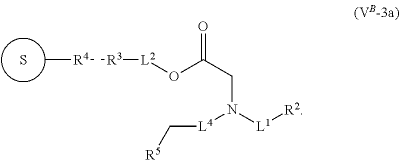

S, L¹, L², R², R³, R⁴, and R⁵ are as described herein, including in embodiments.

In embodiments, the compound has the formula:

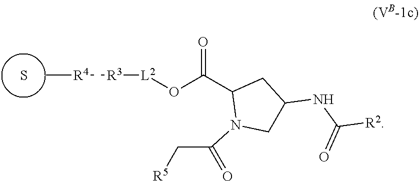

S, L¹, L², R², R³, R⁴, and R⁵ are as described herein, including in embodiments.

In embodiments, the compound has the formula:

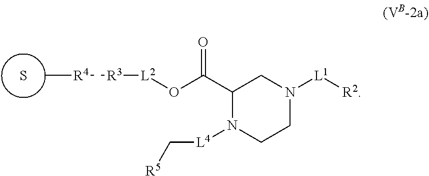

S, L¹, L², L⁴, R², R³, R⁴, and R⁵ are as described herein, including in embodiments.

In embodiments, the compound has the formula:

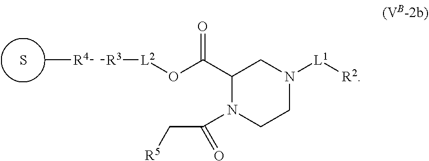

S, L¹, L², R², R³, R⁴, and R⁵ are as described herein, including in embodiments.

In embodiments, the compound has the formula:

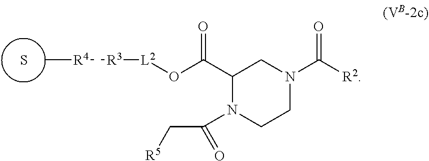

S, L¹, L², R², R³, R⁴, and R⁵ are as described herein, including in embodiments.

In embodiments, the compound has the formula:

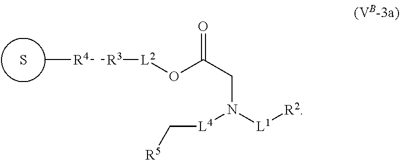

S, L¹, L², L⁴, R², R³, R⁴, and R⁵ are as described herein, including in embodiments.

In embodiments, the compound has the formula:

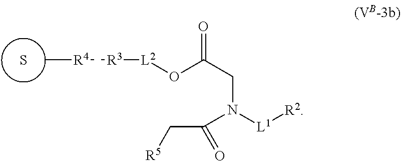

S, L¹, L², R², R³, R⁴, and R⁵ are as described herein, including in embodiments.

In embodiments, the compound has the formula:

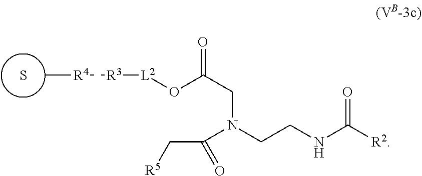

S, L¹, L², R², R³, R⁴, and R⁵ are as described herein, including in embodiments.

In an aspect is provided a compound, or a pharmaceutically acceptable salt thereof, having the formula:

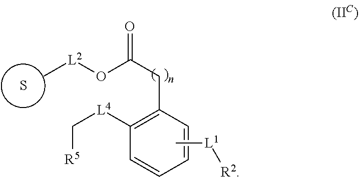

L¹, L⁴, R², and n are as described herein, including in embodiments.

L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is a leaving group.

S is a solid support.

In embodiments, the compound has the formula:

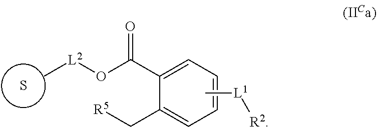

S, L¹, L², R², and R⁵ are as described herein, including in embodiments.

In embodiments, the compound has the formula:

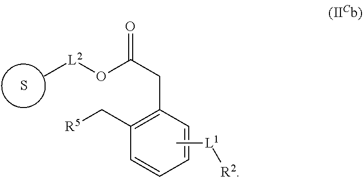

S, L¹, L², R², and R⁵ are as described herein, including in embodiments.

In an aspect is provided a compound, or a pharmaceutically acceptable salt thereof, having the formula:

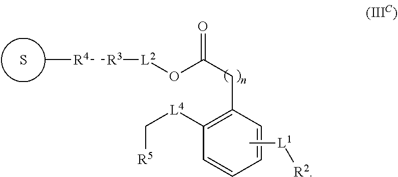

S, L¹, L², L⁴, R², R⁵, and n are as described herein, including in embodiments. R³ and R⁴ are an affinity ligand binding pair. The symbol ---- is a noncovalent or covalent bond.

In embodiments, the compound has the formula:

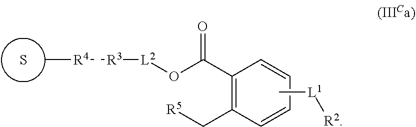

S, L¹, L², R², R³, R⁴, and R⁵ are as described herein, including in embodiments.

In embodiments, the compound has the formula:

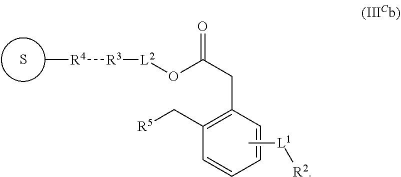

S, L¹, L², R², R³, R⁴, and R⁵ are as described herein, including in embodiments.

In embodiments, ---- is a noncovalent bond. In embodiments, ---- is a covalent bond.

In embodiments, S is a chromatographic material.

In embodiments, solid support refers to any solid or semi-solid material. In embodiments, solid support is an inert, porous solid. In embodiments, the solid support is an active solid. In embodiments, solid support is activated alumina, powdered cellulose, silicic acid, kieselguhr, paper, glass fiber, plastic, agarose, sepharose, silica and derivatives thereof, polymers, or any other suitable solid support.

In embodiments, solid support is glass, synthetic polymer or natural polymer. The solid support may be a column, linear strip, or flow through device. In embodiments, solid support is glass. In embodiments, solid support is synthetic polymer. In embodiments, solid support is natural polymer.

In embodiments, solid support contains functional groups such as carboxyl, amino, aldehyde, alcohol, or other reactive groups that be used to bind other large or small molecules. The binding of the functional group to the solid support or matrix may be achieved directly or through a convenient linker arm. In embodiments, solid support contains carboxyl functional groups. In embodiments, solid support contains amino functional groups. In embodiments, solid support contains aldehyde functional groups. In embodiments, solid support contains alcohol functional groups.

In embodiments, a substituted L² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is a substituted or unsubstituted alkylene or substituted or unsubstituted heteroalkylene. In embodiments, L² is a substituted or unsubstituted C₁-C₄ alkylene. In embodiments, L² is an unsubstituted C₁-C₄ alkylene. In embodiments, L² is an unsubstituted methylene. In embodiments, L² is an unsubstituted ethylene. In embodiments, L² is an unsubstituted propylene. In embodiments, L² is an unsubstituted butylene. In embodiments, L² is a substituted or unsubstituted 2 to 4 membered heteroalkylene. In embodiments, L² is an unsubstituted 2 to 4 membered heteroalkylene.

In embodiments, L² is -L^{2A}-L^{2B}-L^{2C}-L^{2D}-L^{2E}-.

L^{2A}, L^{2B}, L²C, L^{2D}, and L^{2E} are independently a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L^{2A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2A} is substituted with a plurality of groups selected from substituent groups, size-limited substituent groups, and lower substituent groups; each substituent group, size-limited substituent group, and/or lower substituent group may optionally be different. In embodiments, when L^{2A} is substituted, it is substituted with at least one substituent group. In embodiments, when L^{2A} is substituted, it is substituted with at least one size-limited substituent group. In embodiments, when L^{2A} is substituted, it is substituted with at least one lower substituent group.

In embodiments, a substituted L^{2B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2B} is substituted with a plurality of groups selected from substituent groups, size-limited substituent groups, and lower substituent groups; each substituent group, size-limited substituent group, and/or lower substituent group may optionally be different. In embodiments, when L^{2B} is substituted, it is substituted with at least one substituent group. In embodiments, when L^{2B} is substituted, it is substituted with at least one size-limited substituent group. In embodiments, when L^{2B} is substituted, it is substituted with at least one lower substituent group.

In embodiments, a substituted L^{2C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2C} is substituted with a plurality of groups selected from substituent groups, size-limited substituent groups, and lower substituent groups; each substituent group, size-limited substituent group, and/or lower substituent group may optionally be different. In embodiments, when L^{2C} is substituted, it is substituted with at least one substituent group. In embodiments, when L^{2C} is substituted, it is substituted with at least one size-limited substituent group. In embodiments, when L^{2C} is substituted, it is substituted with at least one lower substituent group.

In embodiments, a substituted L^{2D} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2D} is substituted with a plurality of groups selected from substituent groups, size-limited substituent groups, and lower substituent groups; each substituent group, size-limited substituent group, and/or lower substituent group may optionally be different. In embodiments, when L^{2D} is substituted, it is substituted with at least one substituent group. In embodiments, when L^{2D} is substituted, it is substituted with at least one size-limited substituent group. In embodiments, when L^{2D} is substituted, it is substituted with at least one lower substituent group.

In embodiments, a substituted L^{2E} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2E} is substituted with a plurality of groups selected from substituent groups, size-limited substituent groups, and lower substituent groups; each substituent group, size-limited substituent group, and/or lower substituent group may optionally be different. In embodiments, when L^{2E} is substituted, it is substituted with at least one substituent group. In embodiments, when L^{2E} is substituted, it is substituted with at least one size-limited substituent group. In embodiments, when L^{2E} is substituted, it is substituted with at least one lower substituent group.

In embodiments, L^{2A} is a bond, substituted or unsubstituted alkylene, or substituted or unsubstituted heteroalkylene. In embodiments, L^{2A} is a bond. In embodiments, L^{2A} is a substituted or unsubstituted C₂-C₁₂ alkylene. In embodiments, L^{2A} is a substituted or unsubstituted 2 to 12 membered heteroalkylene. In embodiments, L^{2A} is a substituted 2 to 12 membered heteroalkylene. In embodiments, L^{2A} is an oxo-substituted 2 to 12 membered heteroalkylene. In embodiments, L^{2A} is an unsubstituted 2 to 12 membered heteroalkylene.

In embodiments, L^{2B} is a bond, substituted or unsubstituted alkylene, or substituted or unsubstituted heteroalkylene. In embodiments, L^{2B} is a bond. In embodiments, L^{2B} is a substituted or unsubstituted C₂-C₁₂ alkylene. In embodiments, L^{2B} is a substituted or unsubstituted 2 to 12 membered heteroalkylene. In embodiments, L^{2B} is a substituted 2 to 12 membered heteroalkylene. In embodiments, L^{2B} is an oxo-substituted 2 to 12 membered heteroalkylene. In embodiments, L^{2B} is an unsubstituted 2 to 12 membered heteroalkylene.

In embodiments, L^{2C} is a bond or substituted or unsubstituted heteroarylene. In embodiments, L^{2C} is a bond. In embodiments, L^{2C} is a substituted or unsubstituted 5 to 19 membered heteroarylene. In embodiments, L^{2C} is a substituted or unsubstituted 5 to 19 membered fused ring heteroarylene. In embodiments, L^{2C} is an unsubstituted 5 to 19 membered fused ring heteroarylene. In embodiments, L^{2C} is

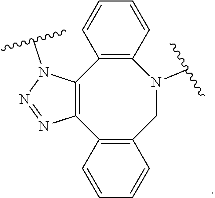 [SMILES: *N1Cc2ccccc2-c2nnn(*)c2-c2ccccc21]

In embodiments, L^{2D} is a bond, substituted or unsubstituted alkylene, or substituted or unsubstituted heteroalkylene. In embodiments, L^{2D} is a bond. In embodiments, L^{2D} is a substituted or unsubstituted C₁-C₄ alkylene. In embodiments, L^{2D} is an unsubstituted C₁-C₄ alkylene. In embodiments, L^{2D} is an unsubstituted methylene. In embodiments, L^{2D} is an unsubstituted ethylene. In embodiments, L^{2D} is an unsubstituted propylene. In embodiments, L^{2D} is an unsubstituted butylene. In embodiments, L^{2D} is a substituted or unsubstituted 2 to 4 membered heteroalkylene. In embodiments, L^{2D} is an unsubstituted 2 to 4 membered heteroalkylene.

In embodiments, L^{2E} is a bond, substituted or unsubstituted alkylene, or substituted or unsubstituted heteroalkylene. In embodiments, L^{2E} is a bond. In embodiments, L^{2E} is a substituted or unsubstituted C₁-C₄ alkylene. In embodiments, L^{2E} is an unsubstituted C₁-C₄ alkylene. In embodiments, L^{2E} is an unsubstituted methylene. In embodiments, L^{2E} is an unsubstituted ethylene. In embodiments, L^{2E} is an unsubstituted propylene. In embodiments, L^{2D} is an unsubstituted butylene. In embodiments, L^{2E} is a substituted or unsubstituted 2 to 4 membered heteroalkylene. In embodiments, L^{2E} is an unsubstituted 2 to 4 membered heteroalkylene.

In embodiments, L² is

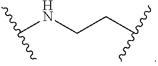 [SMILES: *CCN*]

In embodiments, L² is

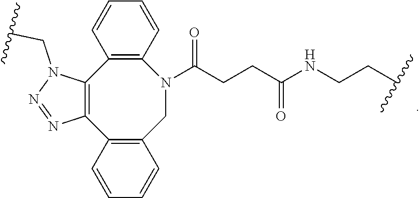 [SMILES: *CCNC(=O)CCC(=O)N1Cc2ccccc2-c2nnn(C*)c2-c2ccccc21]

In embodiments, a substituted R²⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰ is substituted with a plurality of groups selected from substituent groups, size-limited substituent groups, and lower substituent groups; each substituent group, size-limited substituent group, and/or lower substituent group may optionally be different. In embodiments, when R²⁰ is substituted, it is substituted with at least one substituent group. In embodiments, when R²⁰ is substituted, it is substituted with at least one size-limited substituent group. In embodiments, when R²⁰ is substituted, it is substituted with at least one lower substituent group.

In embodiments, R²⁰ is independently hydrogen or substituted or unsubstituted alkyl. In embodiments, R²⁰ is independently hydrogen or substituted or unsubstituted C₁-C₄ alkyl. In embodiments, R²⁰ is independently hydrogen. In embodiments, R²⁰ is independently unsubstituted C₁-C₄ alkyl. In embodiments, R²⁰ is independently unsubstituted methyl. In embodiments, R²⁰ is independently unsubstituted ethyl. In embodiments, R²⁰ is independently unsubstituted propyl. In embodiments, R²⁰ is independently unsubstituted n-propyl. In embodiments, R²⁰ is independently unsubstituted isopropyl. In embodiments, R²⁰ is independently unsubstituted butyl. In embodiments, R²⁰ is independently unsubstituted n-butyl. In embodiments, R²⁰ is independently unsubstituted tert-butyl.

In embodiments, R³ and R⁴ are a biotin-streptavidin binding pair. In embodiments, R³ and R⁴ are a biotin-avidin binding pair. In embodiments, R³ is a monovalent form of biotin. In embodiments, R⁴ is a monovalent form of streptavidin. In embodiments, R⁴ is a monovalent form of avidin.

In embodiments, R⁵ is a halogen,

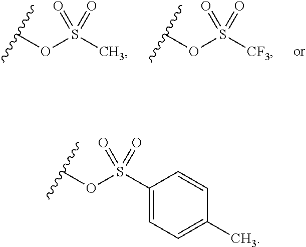 [SMILES: *OS(C)(=O)=O ; *OS(=O)(=O)C(F)(F)F ; *OS(=O)(=O)c1ccc(C)cc1]

In embodiments, R⁵ is a halogen. In embodiments, R⁵ is —F. In embodiments, R⁵ is —Cl. In embodiments, R⁵ is —Br. In embodiments, R⁵ is —I. In embodiments, R⁵ is

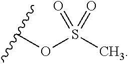 [SMILES: *OS(C)(=O)=O]

In embodiments, R⁵ is

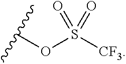 [SMILES: *OS(=O)(=O)C(F)(F)F]

In embodiments, R⁵ is

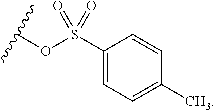 [SMILES: *OS(=O)(=O)c1ccc(C)cc1]

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, Respectively, as Explained in the Definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R¹⁰¹, R¹⁰², and R^{10.3}, respectively.

In embodiments, when R²⁰ is substituted, R²⁰ is substituted with one or more first substituent groups denoted by R^{20.1} as explained in the definitions section above in the description of “first substituent group(s)”. In embodiments, when an R^{20.1} substituent group is substituted, the R^{20.1} substituent group is substituted with one or more second substituent groups denoted by R^{20.2} as explained in the definitions section above in the description of “first substituent group(s)”. In embodiments, when an R^{20.2} substituent group is substituted, the R^{20.2} substituent group is substituted with one or more third substituent groups denoted by R^{20.3} as explained in the definitions section above in the description of “first substituent group(s)”. In the above embodiments, R²⁰, R^{20.1}, R²⁰², and R^{20.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁰, R^{20.1}, R^{20.2}, and R²⁰³, respectively.

In embodiments, when R¹⁰⁰ is substituted, R¹⁰⁰ is substituted with one or more first substituent groups denoted by R^{100.1} as explained in the definitions section above in the description of “first substituent group(s)”. In embodiments, when an R^{100.1} substituent group is substituted, the R^{100.1} substituent group is substituted with one or more second substituent groups denoted by R^{100.2} as explained in the definitions section above in the description of “first substituent group(s)”. In embodiments, when an R^{100.2} substituent group is substituted, the R^{100.2} substituent group is substituted with one or more third substituent groups denoted by R^{100.3} as explained in the definitions section above in the description of “first substituent group(s)”. In the above embodiments, R¹⁰⁰, R^{100.1}, R^{100.2}, and R^{100.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰⁰, R^{100.1}, R^{100.2} and R^{100.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L¹, R^{L1.1}, R^{L1.2}, and R^{L1.3}, respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L², R^{L2.1}, R^{L2.2}, and R^{L2.3}, respectively.

In embodiments, when L^{2A} is substituted, L^{2A} is substituted with one or more first substituent groups denoted by R^{L2A.1} as explained in the definitions section above in the description of “first substituent group(s)”. In embodiments, when an R^{L2A.1} substituent group is substituted, the R^{L2A.1} substituent group is substituted with one or more second substituent groups denoted by R^{L2A.2} as explained in the definitions section above in the description of “first substituent group(s)”. In embodiments, when an R^{L2A.2} substituent group is substituted, the R^{L2A.2} substituent group is substituted with one or more third substituent groups denoted by R^{L2A.3} as explained in the definitions section above in the description of “first substituent group(s)”. In the above embodiments, L²A, R^{L2A.1}, R^{L2A.2}, and R^{L2A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2A}, R^{L2A.1}, R^{L2A.2}, and R^{L2A.3}, respectively.

In embodiments, when L^{2B} is substituted, L^{2B} is substituted with one or more first substituent groups denoted by R^{L2B.1} as explained in the definitions section above in the description of “first substituent group(s)”. In embodiments, when an R^{L2B.1} substituent group is substituted, the R^{L2B.1} substituent group is substituted with one or more second substituent groups denoted by R^{L2B.2} as explained in the definitions section above in the description of “first substituent group(s)”. In embodiments, when an R^{L2B.2} substituent group is substituted, the R^{L2B.2} substituent group is substituted with one or more third substituent groups denoted by R^{L2B.3} as explained in the definitions section above in the description of “first substituent group(s)”. In the above embodiments, L^{2B}, R^{L2B.1}, R^{L2B.2}, and R^{L2B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2B}, R^{L2B.1}, R^{L2B.2}, and R^{L2B.3}, respectively.

In embodiments, when L²C is substituted, L²C is substituted with one or more first substituent groups denoted by R^{L2C.1} as explained in the definitions section above in the description of “first substituent group(s)”. In embodiments, when an R^{L2C.1} substituent group is substituted, the R^{L2C.1} substituent group is substituted with one or more second substituent groups denoted by R^{L2C.2} as explained in the definitions section above in the description of “first substituent group(s)”. In embodiments, when an R^{L2C.2} substituent group is substituted, the R^{L2C.2} substituent group is substituted with one or more third substituent groups denoted by R^{L2C.3} as explained in the definitions section above in the description of “first substituent group(s)”. In the above embodiments, L^{2C}, R^{L2C.1}, R^{L2C.2}, and R^{L2C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2C}, R^{L2C.1}, R^{L2C.2}, and R^{L2C.3}, respectively.

In embodiments, when L^{2D} is substituted, L^{2D} is substituted with one or more first substituent groups denoted by R^{L2D.1} as explained in the definitions section above in the description of “first substituent group(s)”. In embodiments, when an R^{L2D.1} substituent group is substituted, the R^{L2D.1} substituent group is substituted with one or more second substituent groups denoted by R^{L2D.2} as explained in the definitions section above in the description of “first substituent group(s)”. In embodiments, when an R^{L2D.2} substituent group is substituted, the R^{L2D.2} substituent group is substituted with one or more third substituent groups denoted by R^{L2D.3} as explained in the definitions section above in the description of “first substituent group(s)”. In the above embodiments, L^{2D}, R^{L2D.1}, R^{L2D.2}, and R^{L2D.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2D}, R^{L2D.1}, R^{L2D.2}, and R^{L2D.3}, respectively.

In embodiments, when L^{2E} is substituted, L^{2E} is substituted with one or more first substituent groups denoted by R^{L2E.1} as explained in the definitions section above in the description of “first substituent group(s)”. In embodiments, when an R^{L2E.1} substituent group is substituted, the R^{L2E.1} substituent group is substituted with one or more second substituent groups denoted by R^{L2E.2} as explained in the definitions section above in the description of “first substituent group(s)”. In embodiments, when an R^{L2E.2} substituent group is substituted, the R^{L2E.2} substituent group is substituted with one or more third substituent groups denoted by R^{L2E.3} as explained in the definitions section above in the description of “first substituent group(s)”. In the above embodiments, L^{2E}, R^{L2E.1}, R^{L2E.2}, and R^{L2E.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2E}, R^{L2E.1}, R^{L2E.2}, and R^{L2E.3}, respectively.

In embodiments, when L³ is substituted, L³ is substituted with one or more first substituent groups denoted by R^{L3.1} as explained in the definitions section above in the description of “first substituent group(s)”. In embodiments, when an R^{L3.1} substituent group is substituted, the R^{L3.1} substituent group is substituted with one or more second substituent groups denoted by R^{L3.2} as explained in the definitions section above in the description of “first substituent group(s)”. In embodiments, when an R^{L3.2} substituent group is substituted, the R^{L3.2} substituent group is substituted with one or more third substituent groups denoted by R^{L0.3} as explained in the definitions section above in the description of “first substituent group(s)”. In the above embodiments, L³, R^{L3.1}, R^{L3.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L³, R^{L3.1}, R^{L3.2}, and R^{L3.3}, respectively.

In embodiments, when L⁴ is substituted, L⁴ is substituted with one or more first substituent groups denoted by R^{L4.1} as explained in the definitions section above in the description of “first substituent group(s)”. In embodiments, when an R^{L4.1} substituent group is substituted, the R^{L4.1} substituent group is substituted with one or more second substituent groups denoted by R^{L4.2} as explained in the definitions section above in the description of “first substituent group(s)”. In embodiments, when an R^{L4.2} substituent group is substituted, the R^{L4.2} substituent group is substituted with one or more third substituent groups denoted by R^{L4.3} as explained in the definitions section above in the description of “first substituent group(s)”. In the above embodiments, L⁴, R^{L4.1}, R^{L4.2}, and R^{L4.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L⁴, R^{L4.1}, R^{L4.2}, and R^{L4.3}, respectively.

In embodiments, when L⁵ is substituted, L⁵ is substituted with one or more first substituent groups denoted by R^{L5.1} as explained in the definitions section above in the description of “first substituent group(s)”. In embodiments, when an R^{L5.1} substituent group is substituted, the R^{L5.1} substituent group is substituted with one or more second substituent groups denoted by R^{L5.2} as explained in the definitions section above in the description of “first substituent group(s)”. In embodiments, when an R^{L5.2} substituent group is substituted, the R^{L5.2} substituent group is substituted with one or more third substituent groups denoted by R^{L5.3} as explained in the definitions section above in the description of “first substituent group(s)”. In the above embodiments, L⁵, R^{L5.1}, R^{L5.2}, and R^{L5.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L⁵, R^{L5.1}, R^{L5.2}, and R^{L5.3}, respectively.

In embodiments, when L¹⁰ is substituted, L¹⁰ is substituted with one or more first substituent groups denoted by R^{L10.1} as explained in the definitions section above in the description of “first substituent group(s)”. In embodiments, when an R^{L10.1} substituent group is substituted, the R^{L10.1} substituent group is substituted with one or more second substituent groups denoted by R^{L10.2} as explained in the definitions section above in the description of “first substituent group(s)”. In embodiments, when an R^{L10.2} substituent group is substituted, the R^{L10.2} substituent group is substituted with one or more third substituent groups denoted by R^{L10.3} as explained in the definitions section above in the description of “first substituent group(s)”. In the above embodiments, L¹⁰, R^{L10.1}, R^{L10.2}, and R^{L10.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰, R^{L10.1}, R^{L10.2}, and R^{L10.3}, respectively.

In embodiments, the compound is

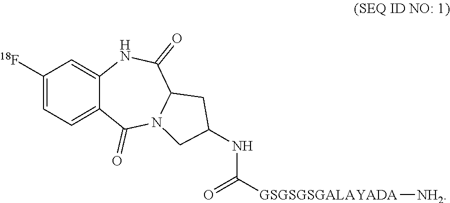

In embodiments, the compound is

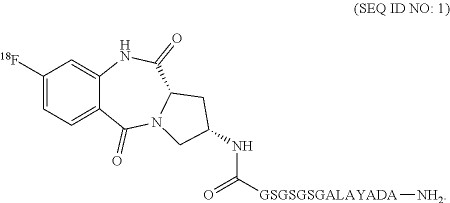

In embodiments, the compound is

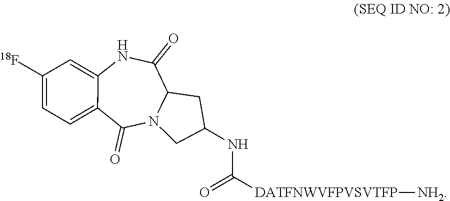

In embodiments, the compound is

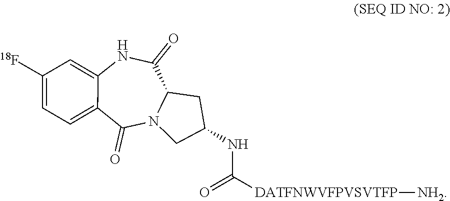

In embodiments, the compound is

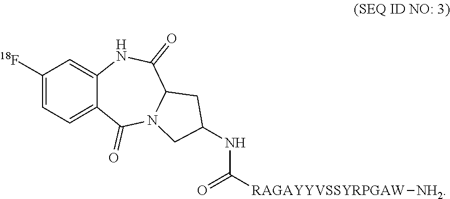

In embodiments, the compound is

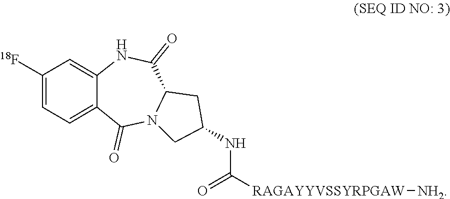

In embodiments, the compound is

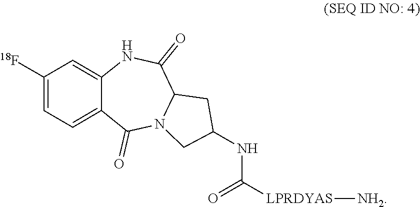

In embodiments, the compound is

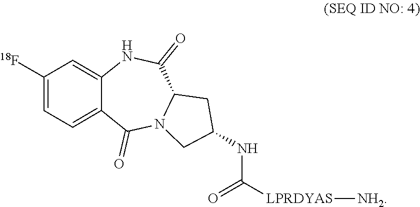

In embodiments, the compound is

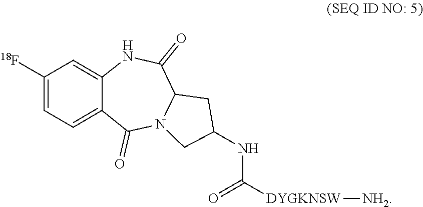

In embodiments, the compound is

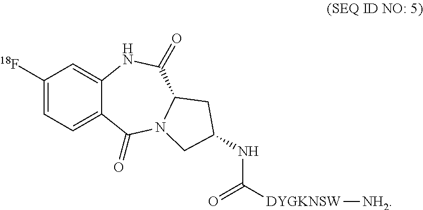

In embodiments, the compound is

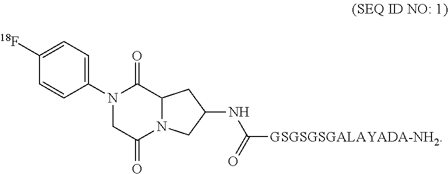

In embodiments, the compound is

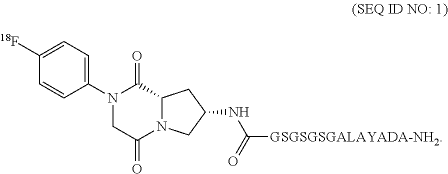

In embodiments, the compound is

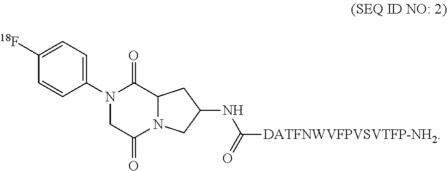

In embodiments, the compound is

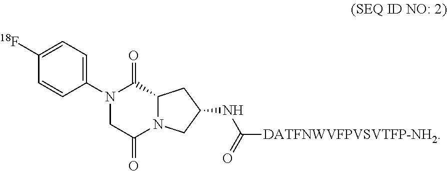

In embodiments, the compound is

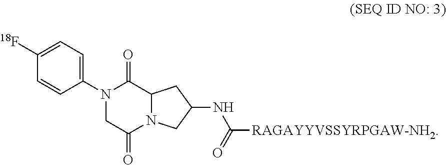

In embodiments, the compound is

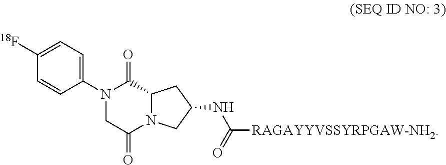

In embodiments, the compound is

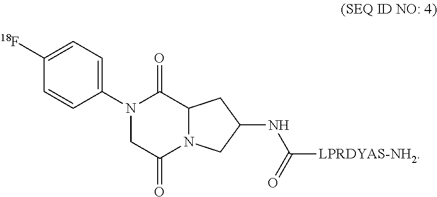

In embodiments, the compound is

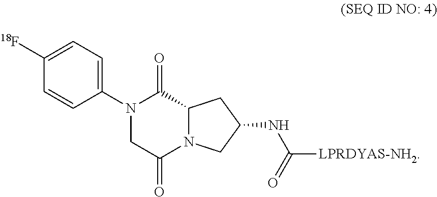

In embodiments, the compound is

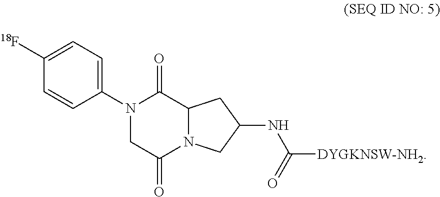

In embodiments, the compound is

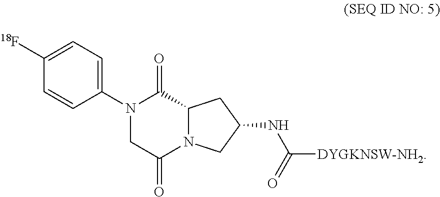

In embodiments, the compound is

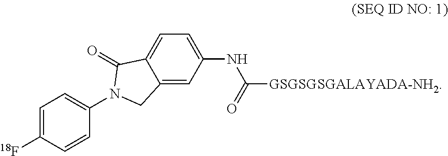

In embodiments, the compound is

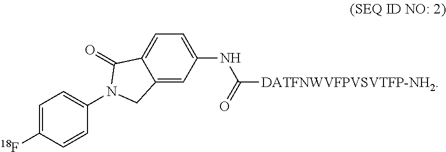

In embodiments, the compound is

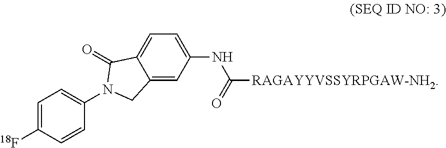

In embodiments, the compound is

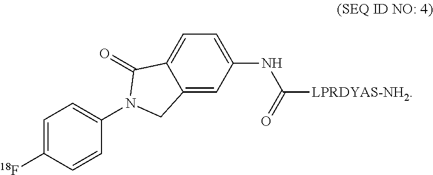

In embodiments, the compound is

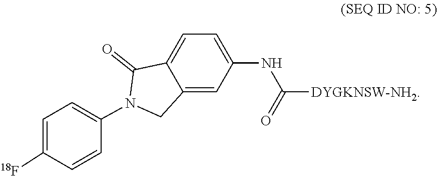

In embodiments, the compound is

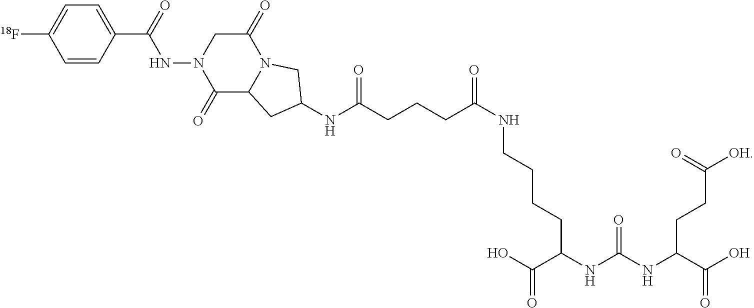 [SMILES: O=C(O)CCC(NC(=O)NC(CCCCNC(=O)CCCC(=O)NC1CC2C(=O)N(NC(=O)c3ccc([18F])cc3)CC(=O)N2C1)C(=O)O)C(=O)O]

In embodiments, the compound is

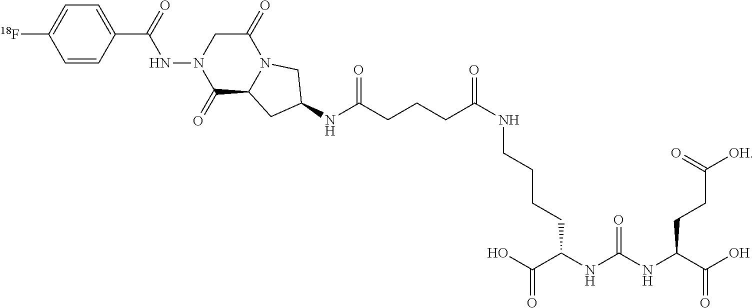 [SMILES: O=C(O)CC[C@H](NC(=O)N[C@@H](CCCCNC(=O)CCCC(=O)N[C@H]1C[C@H]2C(=O)N(NC(=O)c3ccc([18F])cc3)CC(=O)N2C1)C(=O)O)C(=O)O]

In embodiments, the compound is

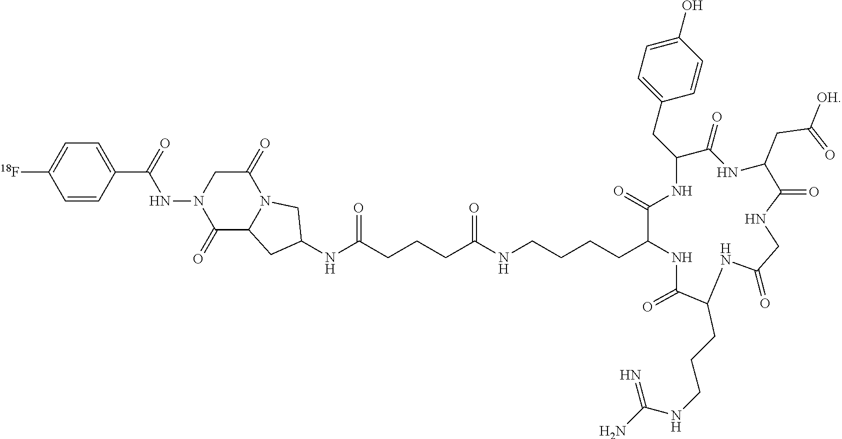 [SMILES: N=C(N)NCCCC1NC(=O)CNC(=O)C(CC(=O)O)NC(=O)C(Cc2ccc(O)cc2)NC(=O)C(CCCCNC(=O)CCCC(=O)NC2CC3C(=O)N(NC(=O)c4ccc([18F])cc4)CC(=O)N3C2)NC1=O]

In embodiments, the compound is

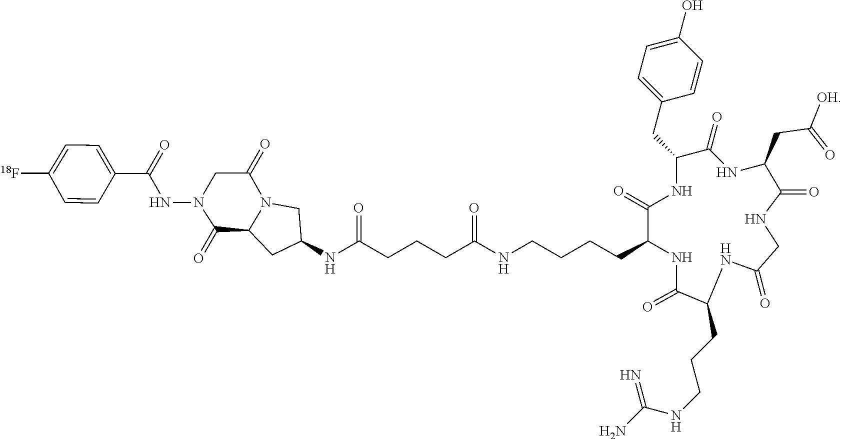 [SMILES: N=C(N)NCCC[C@@H]1NC(=O)CNC(=O)[C@H](CC(=O)O)NC(=O)[C@@H](Cc2ccc(O)cc2)NC(=O)[C@H](CCCCNC(=O)CCCC(=O)N[C@H]2C[C@H]3C(=O)N(NC(=O)c4ccc([18F])cc4)CC(=O)N3C2)NC1=O]

In embodiments, the compound is

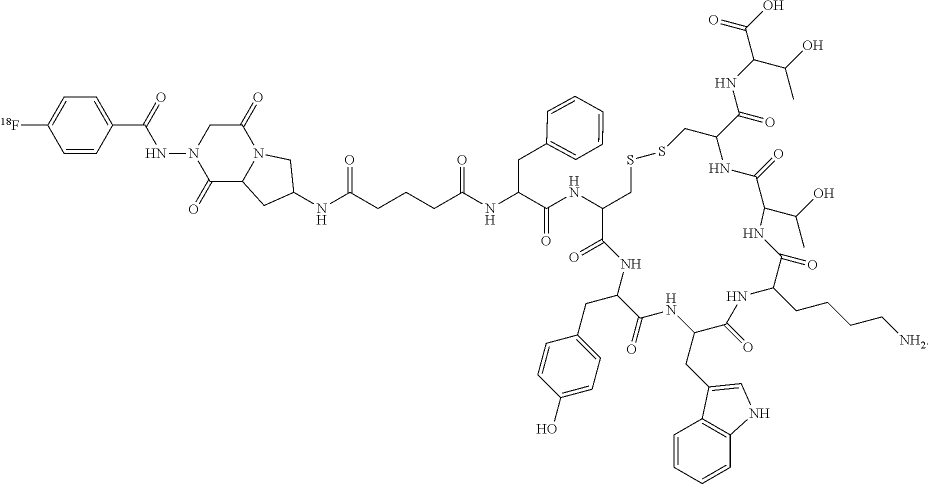 [SMILES: CC(O)C(NC(=O)C1CSSCC(NC(=O)C(Cc2ccccc2)NC(=O)CCCC(=O)NC2CC3C(=O)N(NC(=O)c4ccc([18F])cc4)CC(=O)N3C2)C(=O)NC(Cc2ccc(O)cc2)C(=O)NC(Cc2c[nH]c3ccccc23)C(=O)NC(CCCCN)C(=O)NC(C(C)O)C(=O)N1)C(=O)O]

In embodiments, the compound is

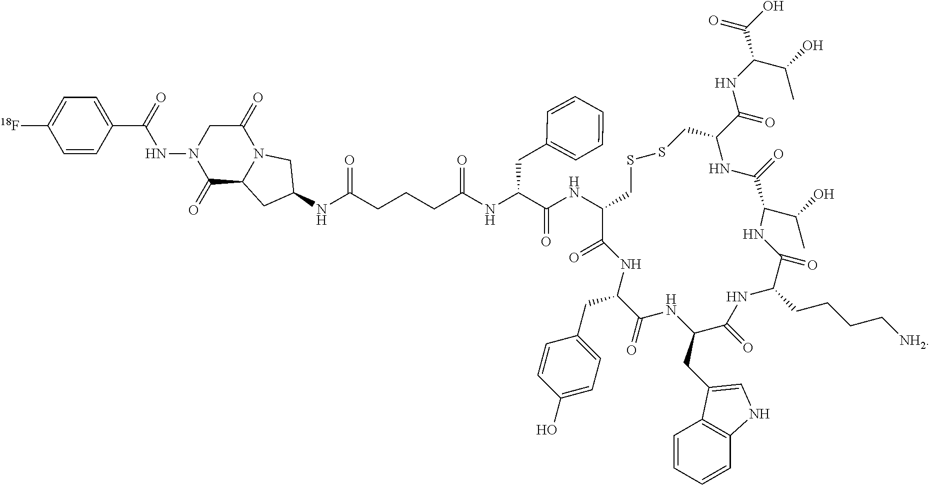 [SMILES: C[C@@H](O)[C@H](NC(=O)[C@H]1CSSC[C@@H](NC(=O)[C@@H](Cc2ccccc2)NC(=O)CCCC(=O)N[C@H]2C[C@H]3C(=O)N(NC(=O)c4ccc([18F])cc4)CC(=O)N3C2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

In embodiments, the compound is

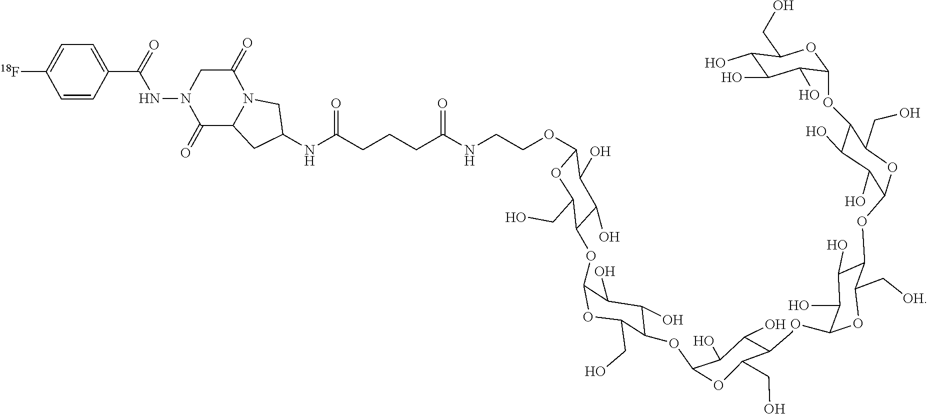 [SMILES: O=C(CCCC(=O)NC1CC2C(=O)N(NC(=O)c3ccc([18F])cc3)CC(=O)N2C1)NCCOC1OC(CO)C(OC2OC(CO)C(OC3OC(CO)C(COC4OC(CO)C(OC5OC(CO)C(OC6OC(CO)C(O)C(O)C6O)C(O)C5O)C(O)C4O)C(O)C3O)C(O)C2O)C(O)C1O]

In embodiments, the compound is

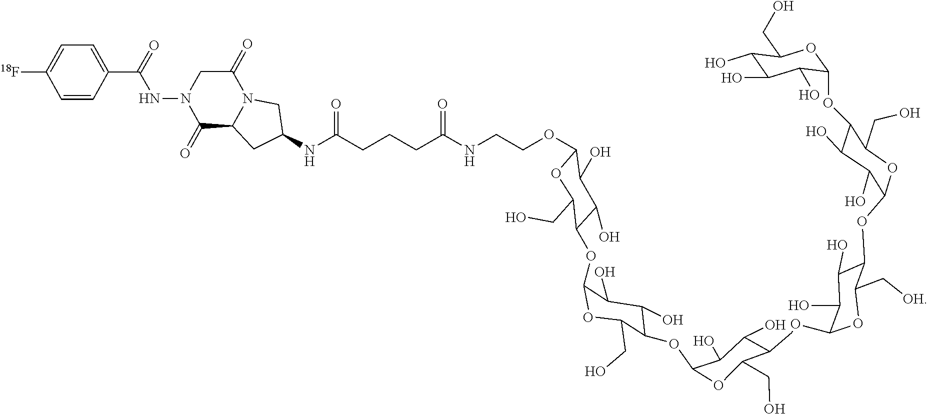 [SMILES: O=C(CCCC(=O)N[C@H]1C[C@H]2C(=O)N(NC(=O)c3ccc([18F])cc3)CC(=O)N2C1)NCCOC1OC(CO)C(OC2OC(CO)C(OC3OC(CO)C(COC4OC(CO)C(OC5OC(CO)C(OC6OC(CO)C(O)C(O)C6O)C(O)C5O)C(O)C4O)C(O)C3O)C(O)C2O)C(O)C1O]

In embodiments, the compound is

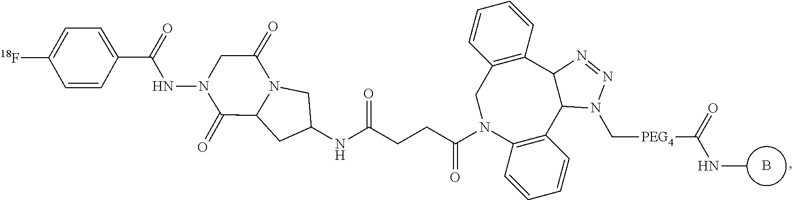

wherein B is a monovalent form of a Her2 receptor binding nanobody.

In embodiments, the compound is

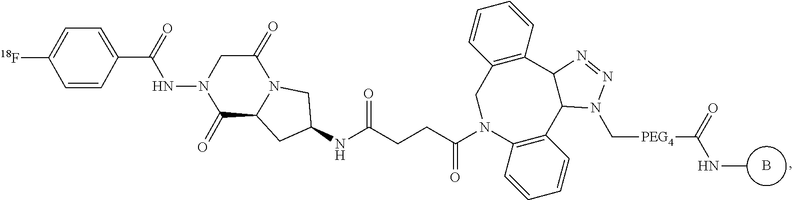

wherein B is a monovalent form of a Her2 receptor binding nanobody.

In embodiments, the compound is

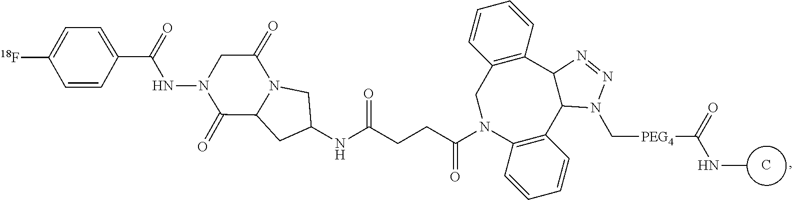

Wherein C is a monovalent form of a TNF-α binding antibody.

In embodiments, the compound is

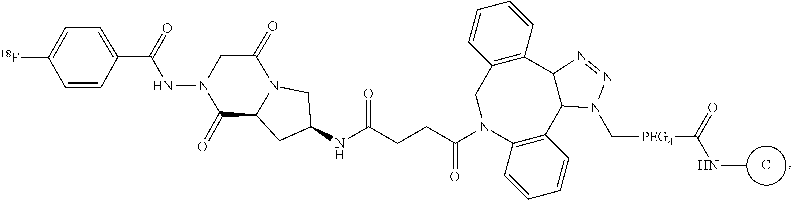

wherein C is a monovalent form of a TNF-α binding antibody.

In embodiments, the compound is useful as a detectable agent. In embodiments, the compound is useful as a positron emission tomography (PET) agent.

In embodiments, the compound is useful as a comparator compound. In embodiments, the comparator compound can be used to assess the activity of a test compound in an assay (e.g., an assay as described herein, for example in the examples section, figures, or tables).

In embodiments, the compound is a compound described herein (e.g., in an aspect, embodiment, example, table, figure, or claim).

### III. Pharmaceutical Compositions

In an aspect is provided a pharmaceutical composition including a compound described herein, or a pharmaceutically acceptable salt thereof, and a pharmaceutically acceptable excipient.

In embodiments, the compound, or pharmaceutically acceptable salt thereof, is included in a therapeutically effective amount.

In embodiments, the pharmaceutical composition includes a second agent (e.g., therapeutic agent). In embodiments, the pharmaceutical composition includes a second agent (e.g., therapeutic agent) in a therapeutically effective amount. In embodiments, the second agent is an anti-cancer agent (e.g., as described herein).

### IV. Methods of Use

In an aspect is provided a method of detecting a level of a compound in a subject, the method including: (i) administering to the subject an effective amount of a compound described herein, or a pharmaceutically acceptable salt thereof, and (ii) detecting the level of the compound in the subject.

In embodiments, step (ii) further includes detecting the level of the compound in the subject using positron emission tomography (PET), wherein R¹ is a PET detectable radioisotope. In embodiments, the method further includes detecting a physiological location of the compound in the subject using PET.

In embodiments, the level of the compound in the subject is indicative of a disease. In embodiments, the level of the compound is elevated relative to a control level. In embodiments, the control level is a level of the compound detected in a subject without the disease. In embodiments, the disease is a cancer (e.g., brain cancer, cervical cancer, colorectal cancer, esophageal cancer, head and neck cancer, Hodgkin's lymphoma, non-Hodgkin's lymphoma, lung cancer, melanoma, pancreatic cancer, prostate cancer, or thyroid cancer). In embodiments, the cancer is prostate cancer. In embodiments, the disease is abnormal brain function. In embodiments, the disease is a neurodegenerative disease (e.g., Alzheimer's disease, brain hemotoma, brain tumor, dementia, epilepsy, Huntington's disease, multiple sclerosis, or Parkinson's disease). In embodiments, the disease is cardiovascular disease (e.g., cardiac infection, cardiac sarcoidosis, congestive heart failure, coronary artery disease, pulmonary embolism, pulmonary sarcoidosis, or stroke). In embodiments, the disease is diabetes.

In embodiments, the subject has cancer (e.g., brain cancer, cervical cancer, colorectal cancer, esophageal cancer, head and neck cancer, Hodgkin's lymphoma, non-Hodgkin's lymphoma, lung cancer, melanoma, pancreatic cancer, prostate cancer, or thyroid cancer). In embodiments, the subject has abnormal brain function. In embodiments, the subject has a neurodegenerative disease (e.g., Alzheimer's disease, brain hemotoma, brain tumor, dementia, epilepsy, Huntington's disease, multiple sclerosis, or Parkinson's disease). In embodiments, the subject has cardiovascular disease (e.g., cardiac infection, cardiac sarcoidosis, congestive heart failure, coronary artery disease, pulmonary embolism, pulmonary sarcoidosis, or stroke). In embodiments, the subject has diabetes.

In an aspect is provided a method of detecting the level of CD44v6 in a subject, the method including administering to the subject an effective amount of a compound described herein, or a pharmaceutically acceptable salt thereof.

In embodiments, the method further includes detecting the level of the compound using positron emission tomography.

### V. Methods of Making

In an aspect is provided a method of making compound (I^{A}), or a pharmaceutically acceptable salt thereof, the method including mixing compound (II^{A}) or compound (III^{A}) in a reaction vessel; wherein compound (I^{A}) has the formula:

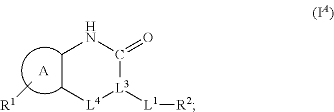

compound (II^{A}) has the formula:

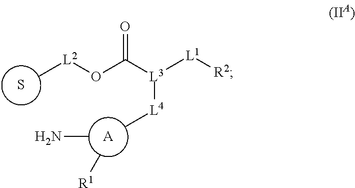

and compound (III^{A}) has the formula:

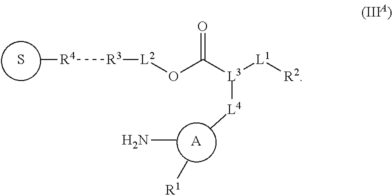

Ring A, S, L¹, L², L³, L⁴, R¹, R², R³, and R⁴ are as described herein, including in embodiments. The symbol ---- is a noncovalent or covalent bond.

In embodiments, the method further includes mixing compound (II^{A}) or compound (III^{A}) in a basic solution. In embodiments, the method further includes mixing compound (II^{A}) in a basic solution. In embodiments, the method further includes mixing compound (III^{A}) in a basic solution. In embodiments, the basic solution has a pH from about 7.1 to about 8. In embodiments, the basic solution has a pH from about 7.1 to about 7.8. In embodiments, the basic solution has a pH from about 7.1 to about 7.6. In embodiments, the basic solution has a pH from about 7.2 to about 7.5. In embodiments, the basic solution has a pH from about 7.3 to about 7.5. In embodiments, the basic solution has a pH of about 7.3. In embodiments, the basic solution has a pH of about 7.4. In embodiments, the basic solution has a pH of about 7.5. In embodiments, the basic solution has a pH from 7.1 to 8. In embodiments, the basic solution has a pH from 7.1 to 7.8. In embodiments, the basic solution has a pH from 7.1 to 7.6. In embodiments, the basic solution has a pH from 7.2 to 7.5. In embodiments, the basic solution has a pH from 7.3 to 7.5. In embodiments, the basic solution has a pH of 7.3. In embodiments, the basic solution has a pH of 7.4. In embodiments, the basic solution has a pH of 7.5.

In embodiments, compound (I^{A}) has the formula:

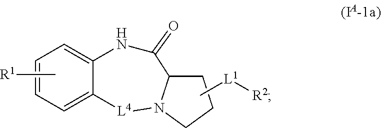

compound (II^{A}) has the formula:

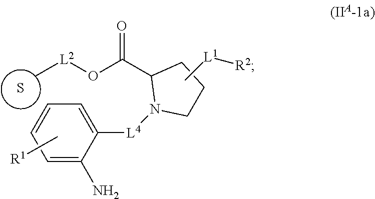

and compound (III^{A}) has the formula:

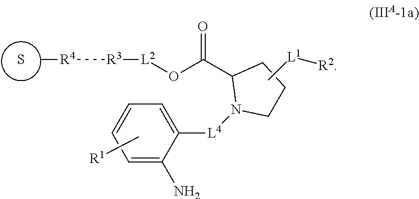

S, L¹, L², L⁴, R¹, R², R³, and R⁴ are as described herein, including in embodiments.

In embodiments, compound (I^{A}) has the formula:

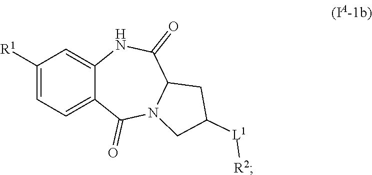

compound (II^{A}) has the formula:

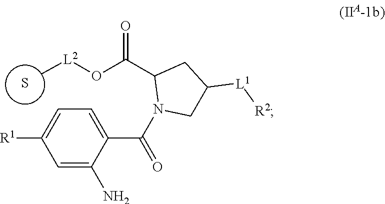

and compound (III^{A}) has the formula:

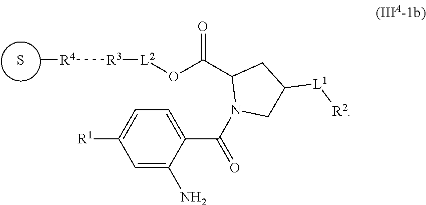

S, L¹, L², R¹, R², R³, and R⁴ are as described herein, including in embodiments.

In embodiments, compound (I^{A}) has the formula:

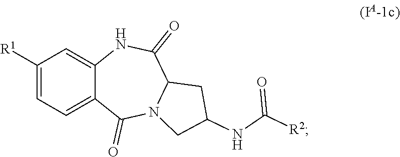

compound (II^{A}) has the formula:

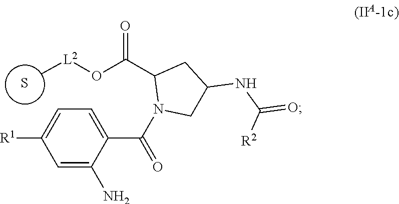

and compound (III^{A}) has the formula:

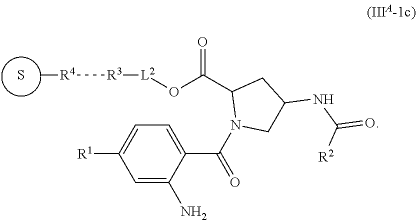

S, L², R¹, R², R³, and R⁴ are as described herein, including in embodiments.

In embodiments, compound (I^{A}) has the formula:

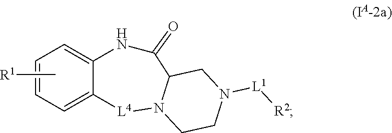

compound (II^{A}) has the formula:

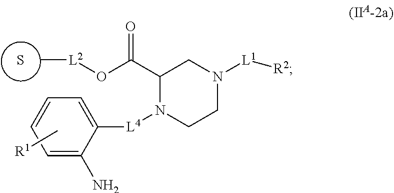

and compound (III^{A}) has the formula:

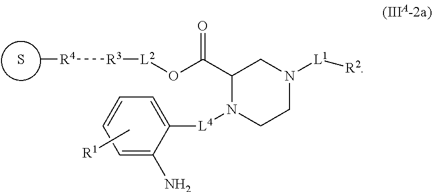

S, L¹, L², L⁴, R¹, R², R³, and R⁴ are as described herein, including in embodiments.

In embodiments, compound (I^{A}) has the formula:

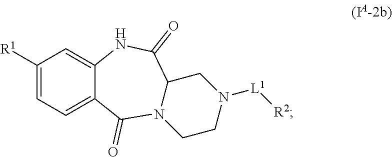

compound (II^{A}) has the formula:

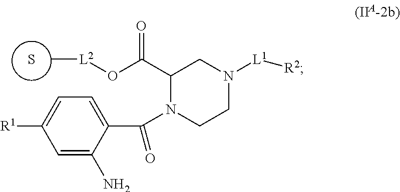

and compound (II^{A}) has the formula:

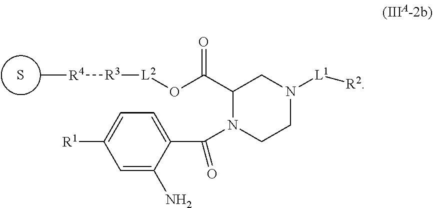

S, L¹, L², R¹, R², R³, and R⁴ are as described herein, including in embodiments.

In embodiments, compound (I^{A}) has the formula:

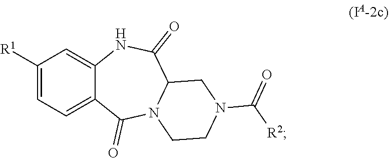

compound (II^{A}) has the formula:

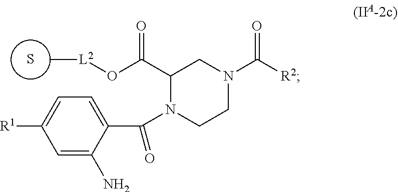

and compound (III^{A}) has the formula:

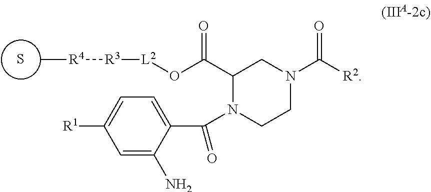

S, L², R¹, R², R³, and R⁴ are as described herein, including in embodiments.

In embodiments, compound (I^{A}) has the formula:

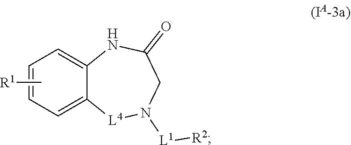

compound (II^{A}) has the formula:

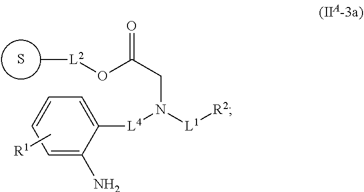

and compound (III^{A}) has the formula:

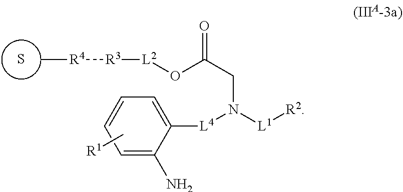

S, L¹, L², L⁴, R¹, R², R³, and R⁴ are as described herein, including in embodiments.

In embodiments, compound (I^{A}) has the formula:

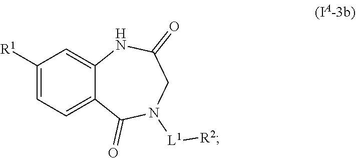

compound (II^{A}) has the formula:

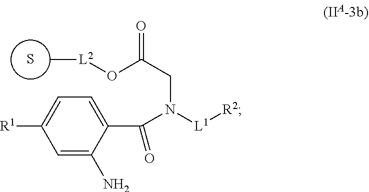

and compound (III^{A}) has the formula:

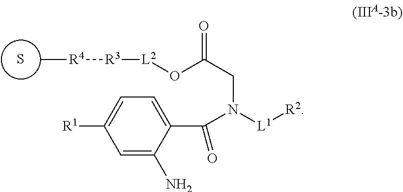

S, L¹, L², R¹, R², R³, and R⁴ are as described herein, including in embodiments.

In embodiments, compound (I^{A}) has the formula:

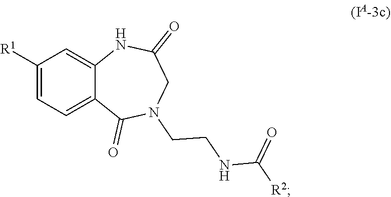

compound (II^{A}) has the formula:

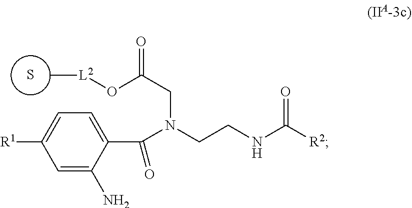

and compound (III^{A}) has the formula:

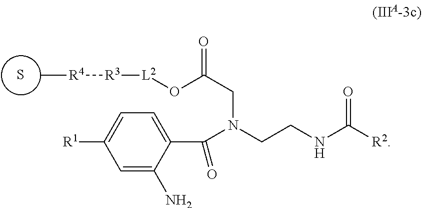

S, L², R¹, R², R³, and R⁴ are as described herein, including in embodiments.

In embodiments, compound (I^{A}) is

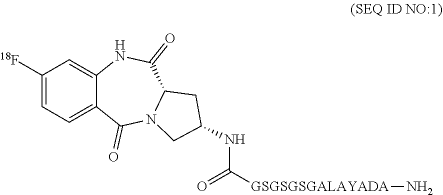

and compound (II^{A}) is

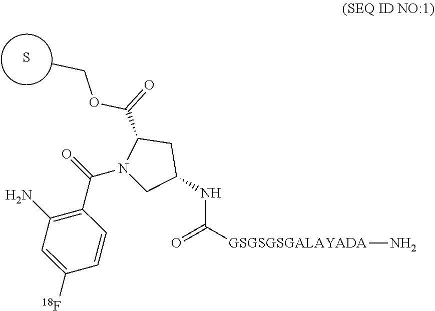

S is a solid support as described herein, including in embodiments.

In embodiments, compound (I^{A}) is

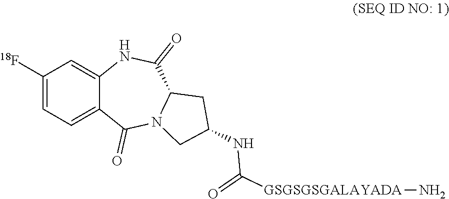

and compound (II^{A}) is

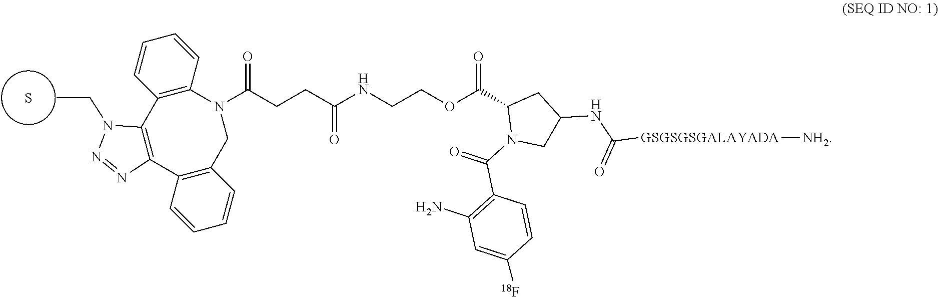

S is a solid support as described herein, including in embodiments.

In embodiments, compound (I^{A}) is

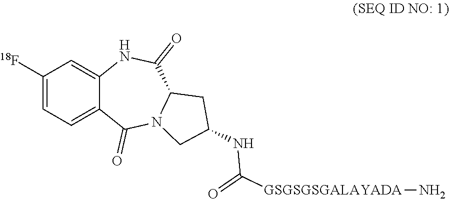

and compound (III^{A}) is

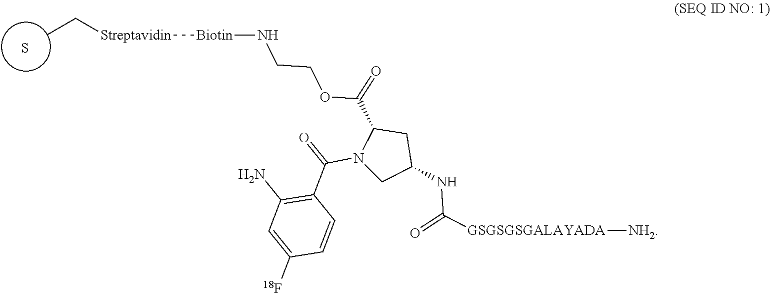

S is a solid support as described herein, including in embodiments.

In embodiments, compound (I^{A}) is

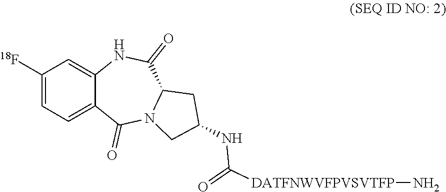

and compound (II^{A}) is

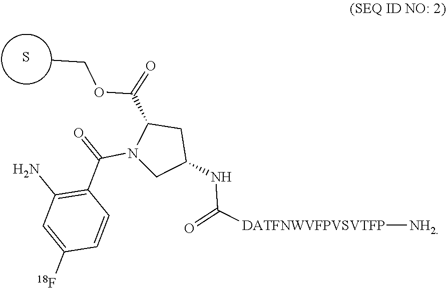

S is a solid support as described herein, including in embodiments.

In embodiments, compound (I^{A}) is

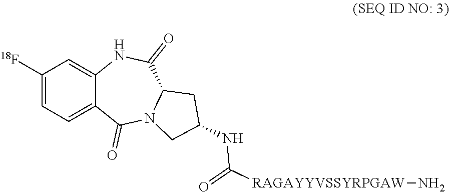

and compound (II^{A}) is

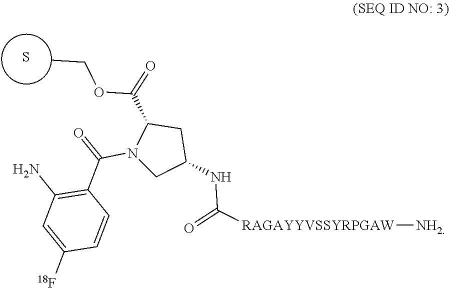

S is a solid support as described herein, including in embodiments.

In embodiments, compound (I^{A}) is

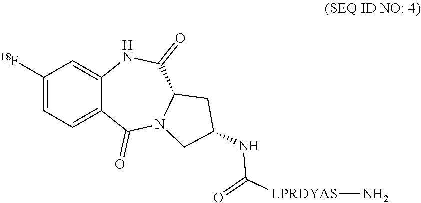

and compound (III^{A}) is

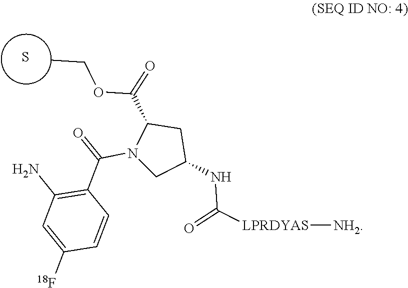

S is a solid support as described herein, including in embodiments.

In embodiments, compound (I^{A}) is

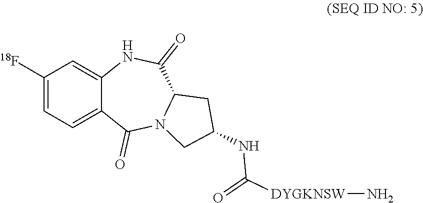

and compound (II^{A}) is

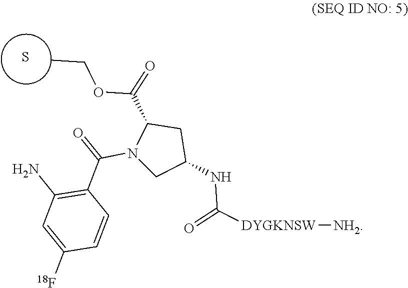

S is a solid support as described herein, including in embodiments.

In embodiments, the method of making compound (I^{A}) includes mixing compound (II^{A}) in a reaction vessel. In embodiments, the method of making compound (I^{A}) includes mixing compound (III^{A}) in a reaction vessel.

In an aspect is provided a method of making compound (I^{B}), or a pharmaceutically acceptable salt thereof, the method including mixing compound (II^{B}) or compound (III^{B}) in a reaction vessel; wherein compound (I^{B}) has the formula:

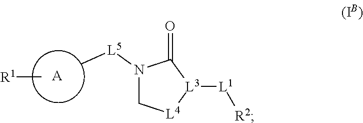

compound (II^{B}) has the formula:

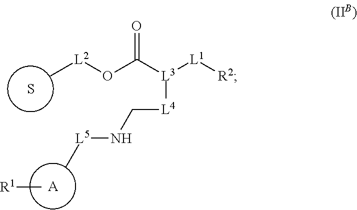

and compound (III^{B}) has the formula:

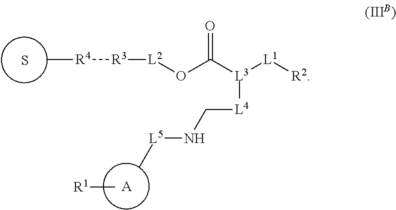

Ring A, S, L¹, L², L³, L⁴, L⁵, R¹, R², R³, and R⁴ are as described herein, including in embodiments. The symbol ---- is a noncovalent or covalent bond.

In embodiments, the method further includes mixing compound (II^{B}) or compound (III^{B}) in a basic solution. In embodiments, the method further includes mixing compound (II^{B}) in a basic solution. In embodiments, the method further includes mixing compound (III^{B}) in a basic solution. In embodiments, the basic solution has a pH from about 7.1 to about 8. In embodiments, the basic solution has a pH from about 7.1 to about 7.8. In embodiments, the basic solution has a pH from about 7.1 to about 7.6. In embodiments, the basic solution has a pH from about 7.2 to about 7.5. In embodiments, the basic solution has a pH from about 7.3 to about 7.5. In embodiments, the basic solution has a pH of about 7.3. In embodiments, the basic solution has a pH of about 7.4. In embodiments, the basic solution has a pH of about 7.5. In embodiments, the basic solution has a pH from 7.1 to 8. In embodiments, the basic solution has a pH from 7.1 to 7.8. In embodiments, the basic solution has a pH from 7.1 to 7.6. In embodiments, the basic solution has a pH from 7.2 to 7.5. In embodiments, the basic solution has a pH from 7.3 to 7.5. In embodiments, the basic solution has a pH of 7.3. In embodiments, the basic solution has a pH of 7.4. In embodiments, the basic solution has a pH of 7.5.

In embodiments, compound (I^{B}) has the formula:

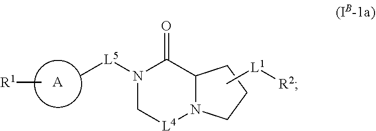

compound (II^{B}) has the formula:

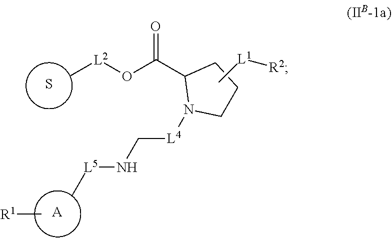

and compound (III^{B}) has the formula:

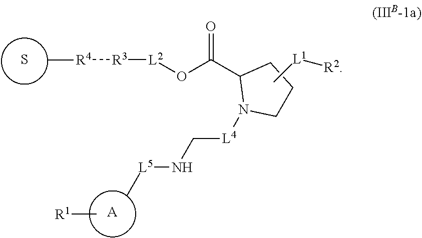

Ring A, S, L¹, L², L⁴, L⁵, R¹, R², R³, and R⁴ are as described herein, including in embodiments.

In embodiments, compound (I^{B}) has the formula:

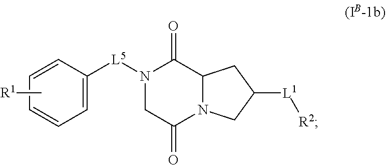

compound (II^{B}) has the formula:

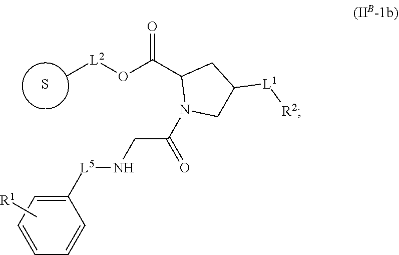

and compound (III^{B}) has the formula:

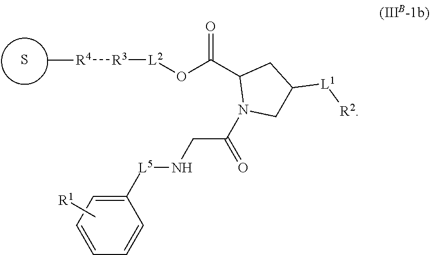

S, L¹, L², L⁵, R¹, R², R³, and R⁴ are as described herein, including in embodiments.

In embodiments, compound (I^{B}) has the formula:

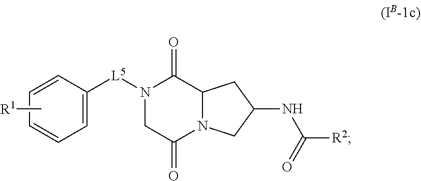

compound (II^{B}) has the formula:

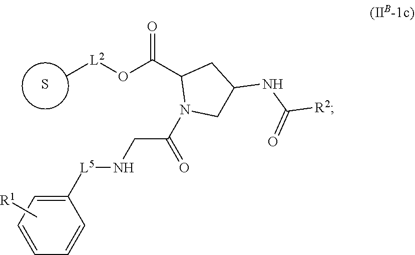

and compound (III^{B}) has the formula:

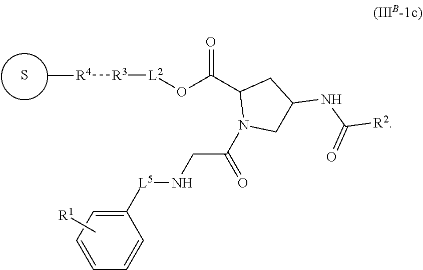

S, L², L⁵, R¹, R², R³, and R⁴ are as described herein, including in embodiments.

In embodiments, compound (I^{B}) has the formula:

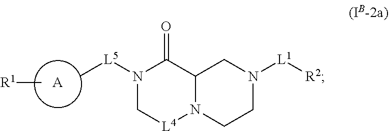

compound (II^{B}) has the formula:

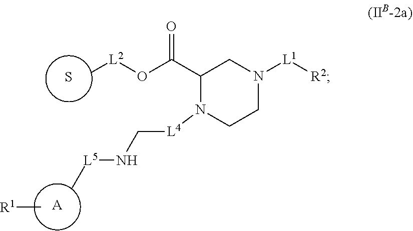

and
compound (III^{B}) has the formula:

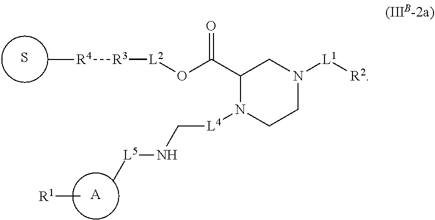

Ring A, S, L¹, L², L⁴, L⁵, R¹, R², R³, and R are as described herein, including in embodiments.

In embodiments, compound (I^{B}) has the formula:

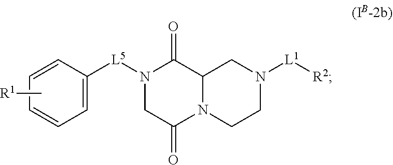

compound (II^{B}) has the formula:

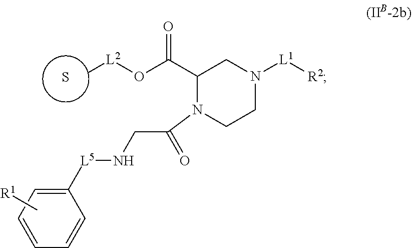

and compound (III^{B}) has the formula:

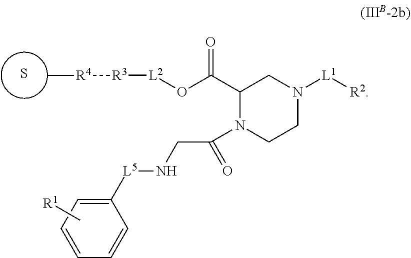

S, L¹, L², L⁵, R¹, R², R³, and R⁴ are as described herein, including in embodiments.

In embodiments, compound (I^{B}) has the formula:

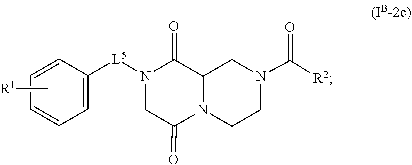

compound (II^{B}) has the formula:

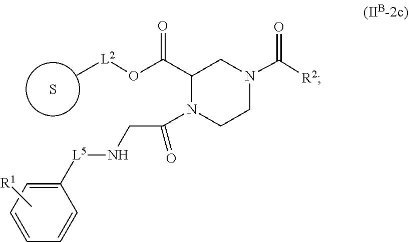

and compound (III^{B}) has the formula:

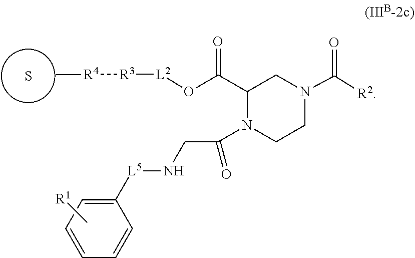

S, L², L⁵, R¹, R², R³, and R⁴ are as described herein, including in embodiments.

In embodiments, compound (I^{B}) has the formula:

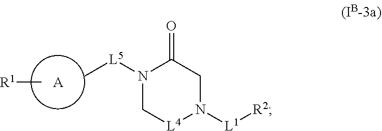

compound (II^{B}) has the formula:

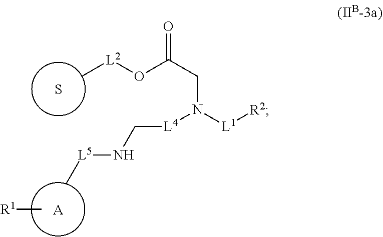

and compound (III^{B}) has the formula:

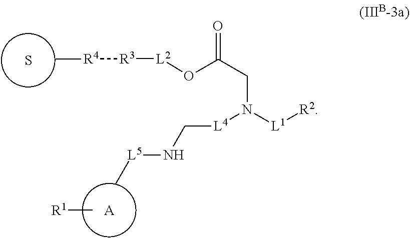

Ring A, S, L¹, L², L⁴, L⁵, R¹, R², R³, and R⁴ are as described herein, including in embodiments.

In embodiments, compound (I^{B}) has the formula:

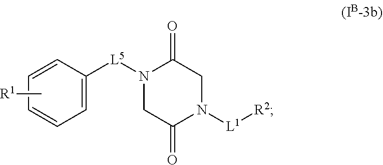

compound (II^{B}) has the formula:

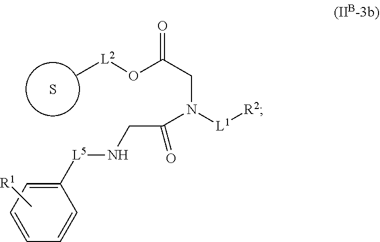

and compound (II^{B}) has the formula:

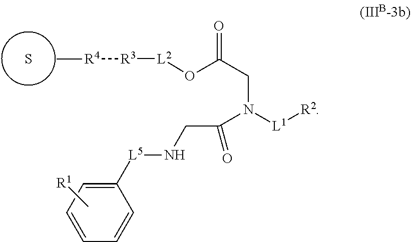

S, L¹, L², L⁵, R¹, R², R³, and R⁴ are as described herein, including in embodiments.

In embodiments, compound (I^{B}) has the formula:

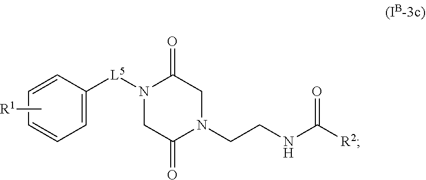

compound (II^{B}) has the formula:

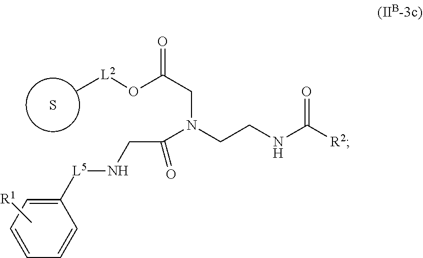

and compound (III^{B}) has the formula:

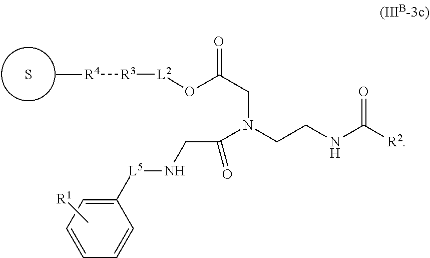

S, L², L⁵, R¹, R², R³, and R⁴ are as described herein, including in embodiments.

In embodiments, compound (I^{B}) is

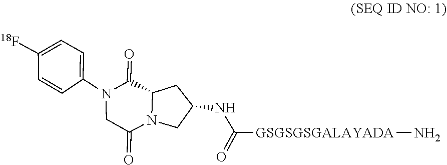

and compound (II^{B}) is

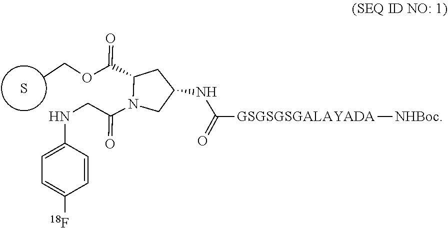

S is a solid support as described herein, including in embodiments.

In embodiments, compound (I^{B}) is

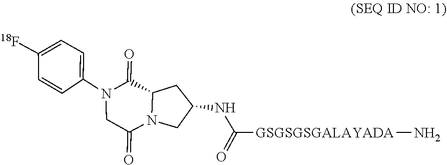

and compound (III^{B}) is

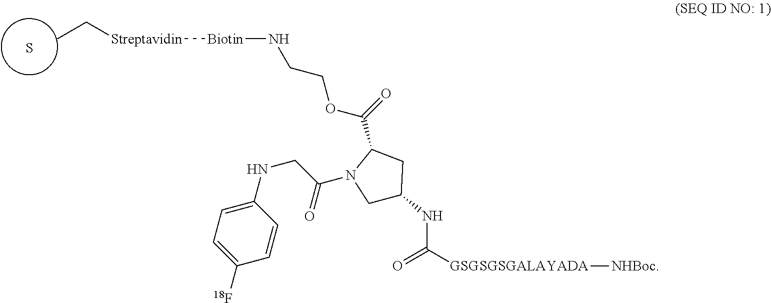

S is a solid support as described herein, including in embodiments.

In embodiments, compound (I^{B}) is

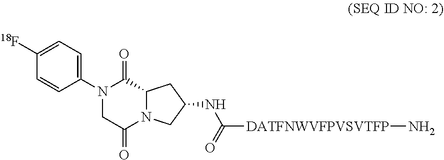

and compound (II^{B}) is

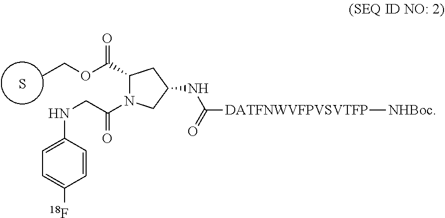

S is a solid support as described herein, including in embodiments.

In embodiments, compound (I^{B}) is

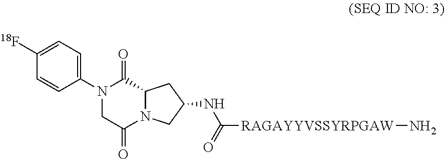

and compound (II^{B}) is

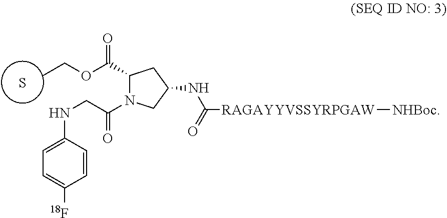

S is a solid support as described herein, including in embodiments.

In embodiments, compound (I^{B}) is

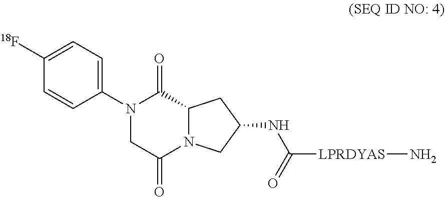

and compound (II^{B}) is

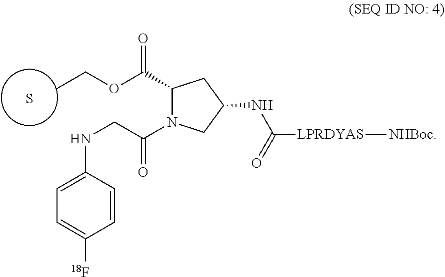

S is a solid support as described herein, including in embodiments.

In embodiments, compound (I^{B}) is

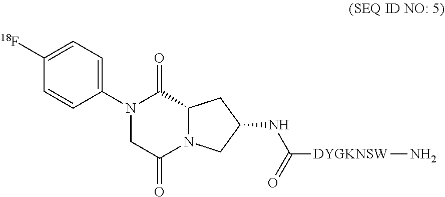

and compound (II^{B}) is

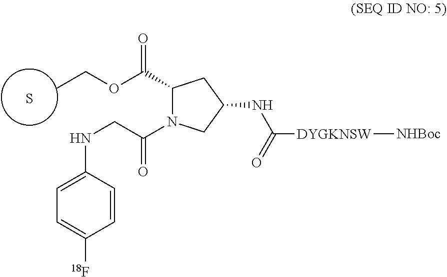

S is a solid support as described herein, including in embodiments.

In embodiments, the method of making compound (I^{B}) includes mixing compound (II^{B}) in a reaction vessel. In embodiments, the method of making compound (I^{B}) includes mixing compound (III^{B}) in a reaction vessel.

In an aspect is provided a method of making compound (I^{B}), or a pharmaceutically acceptable salt thereof, the method including mixing compound (IV^{B}) or compound (V^{B}) and compound (A) in a reaction vessel; wherein compound (I^{B}) has the formula:

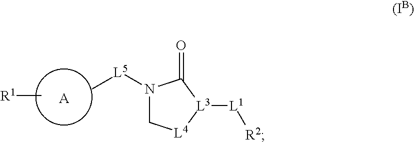

compound (IV^{B}) has the formula:

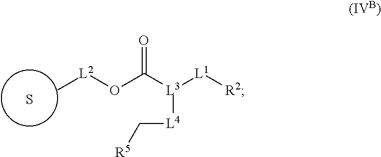

compound (V^{B}) has the formula:

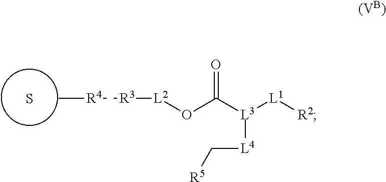

and compound (A) has the formula:

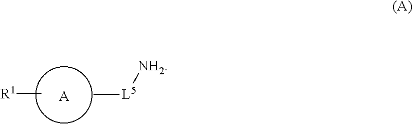

Ring A, S, L¹, L², L³, L⁴, L⁵, R¹, R², R³, R⁴, and R⁵ are as described herein, including in embodiments. The symbol ---- is a noncovalent or covalent bond.

In embodiments, compound (I^{B}) has the formula:

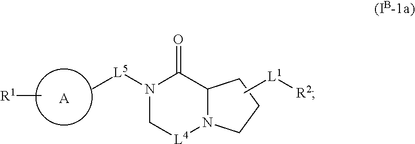

compound (IV^{B}) has the formula:

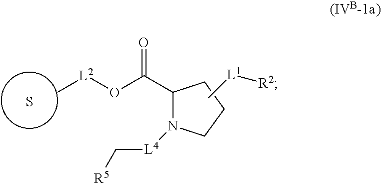

and compound (V^{B}) has the formula:

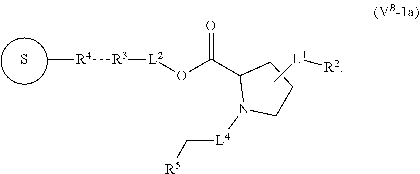

Ring A, S, L¹, L², L⁴, L⁵, R¹, R², R³, R⁴, and R⁵ are as described herein, including in embodiments.

In embodiments, compound (I^{B}) has the formula:

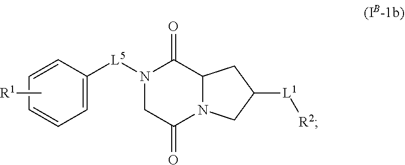

compound (IV^{B}) has the formula:

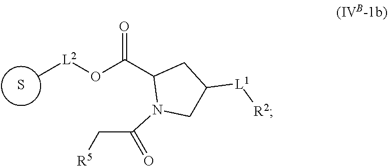

and compound (V^{B}) has the formula:

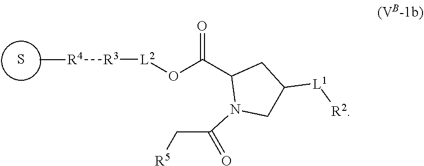

S, L¹, L², L⁵, R¹, R², R³, R⁴, and R⁵ are as described herein, including in embodiments.

In embodiments, compound (I^{B}) has the formula:

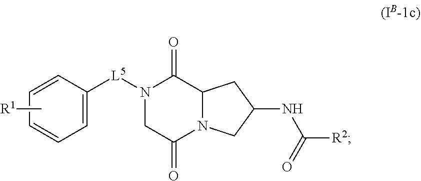

compound (IV^{B}) has the formula:

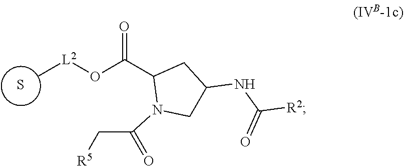

and compound (V^{B}) has the formula:

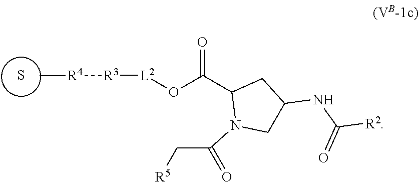

S, L², L⁵, R¹, R², R³, R⁴, and R⁵ are as described herein, including in embodiments.

In embodiments, compound (I^{B}) has the formula:

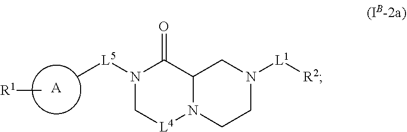

compound (IV^{B}) has the formula:

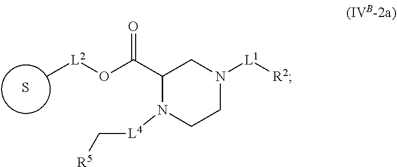

and compound (V^{B}) has the formula:

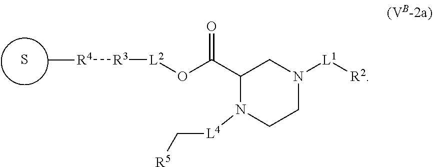

Ring A, S, L¹, L², L⁴, L⁵, R¹, R², R³, R⁴, and R⁵ are as described herein, including in embodiments.

In embodiments, compound (I^{B}) has the formula:

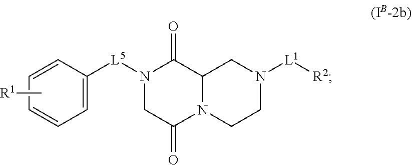

compound (IV^{B}) has the formula:

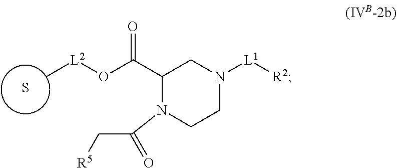

and compound (V^{B}) has the formula:

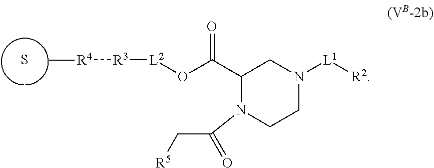

S, L¹, L², L⁵, R¹, R², R³, R⁴, and R⁵ are as described herein, including in embodiments.

In embodiments, compound (I^{B}) has the formula:

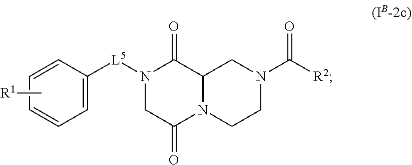

compound (IV^{B}) has the formula:

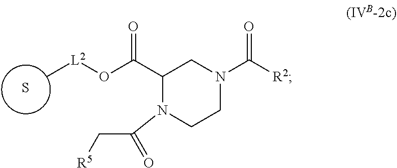

and compound (V^{B}) has the formula:

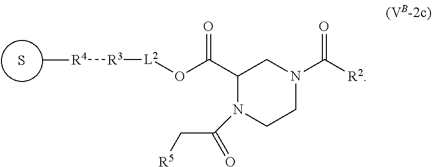

S, L², L⁵, R¹, R², R³, and R⁴ are as described herein, including in embodiments.

In embodiments, compound (I^{B}) has the formula:

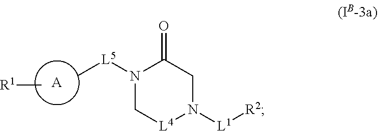

compound (IV^{B}) has the formula:

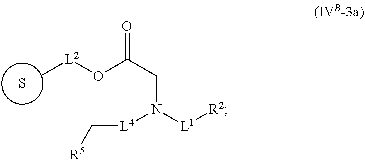

and compound (V^{B}) has the formula:

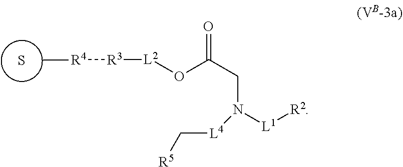

Ring A, S, L¹, L², L⁴, L⁵, R¹, R², R³, R⁴, and R⁵ are as described herein, including in embodiments.

In embodiments, compound (I^{B}) has the formula:

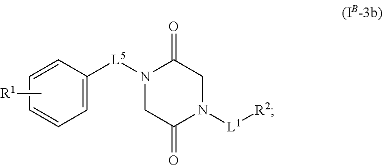

compound (IV^{B}) has the formula:

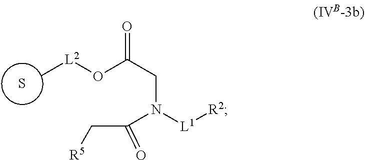

and compound (V^{B}) has the formula:

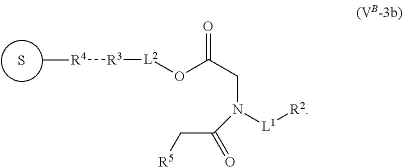

S, L¹, L², L⁵, R¹, R², R³, R⁴, and R⁵ are as described herein, including in embodiments.

In embodiments, compound (I^{B}) has the formula:

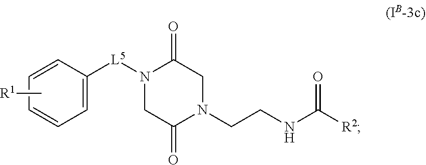

compound (IV^{B}) has the formula:

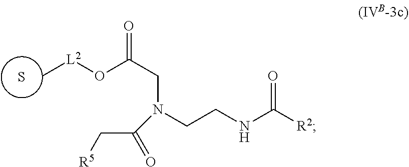

and compound (V^{B}) has the formula:

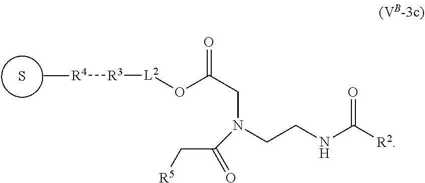

S, L², L⁵, R¹, R², R³, R⁴, and R⁵ are as described herein, including in embodiments.

In embodiments, compound (I^{B}) is

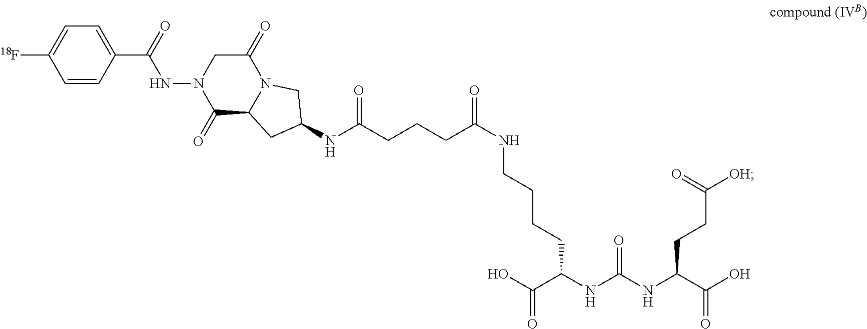 [SMILES: O=C(O)CC[C@H](NC(=O)N[C@@H](CCCCNC(=O)CCCC(=O)N[C@H]1C[C@H]2C(=O)N(NC(=O)c3ccc([18F])cc3)CC(=O)N2C1)C(=O)O)C(=O)O]

is

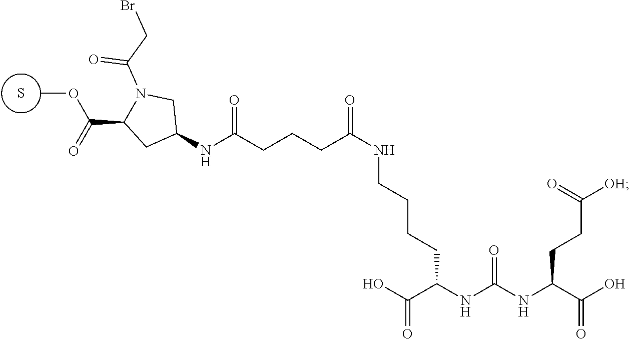

and compound (A) is

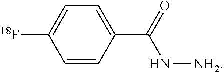 [SMILES: NNC(=O)c1ccc([18F])cc1]

S is a solid support as described herein, including in embodiments.

In embodiments, compound (I^{B}) is

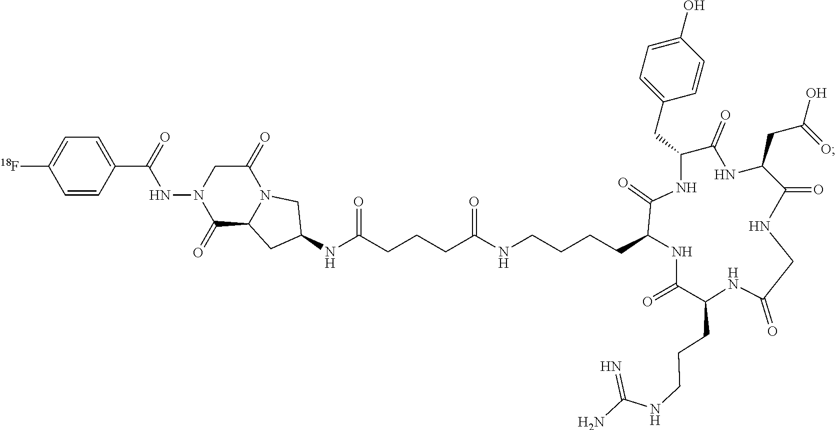 [SMILES: N=C(N)NCCC[C@@H]1NC(=O)CNC(=O)[C@H](CC(=O)O)NC(=O)[C@@H](Cc2ccc(O)cc2)NC(=O)[C@H](CCCCNC(=O)CCCC(=O)N[C@H]2C[C@H]3C(=O)N(NC(=O)c4ccc([18F])cc4)CC(=O)N3C2)NC1=O]

compound (IV^{B}) is

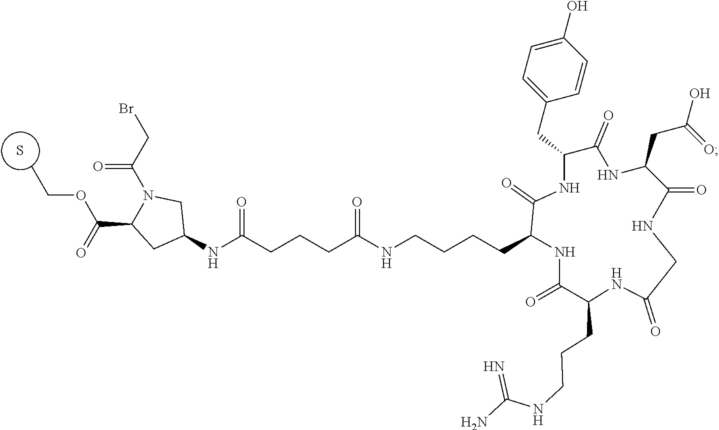

and compound (A) is

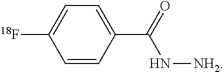 [SMILES: NNC(=O)c1ccc([18F])cc1]

S is a solid support as described herein, including in embodiments.

In embodiments, compound (I^{B}) is

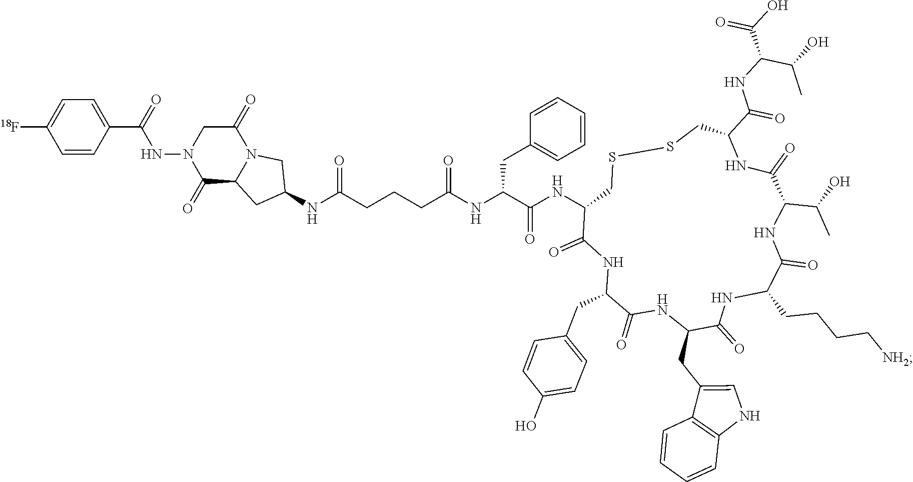 [SMILES: C[C@@H](O)[C@H](NC(=O)[C@H]1CSSC[C@@H](NC(=O)[C@@H](Cc2ccccc2)NC(=O)CCCC(=O)N[C@H]2C[C@H]3C(=O)N(NC(=O)c4ccc([18F])cc4)CC(=O)N3C2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

compound (IV^{B}) is

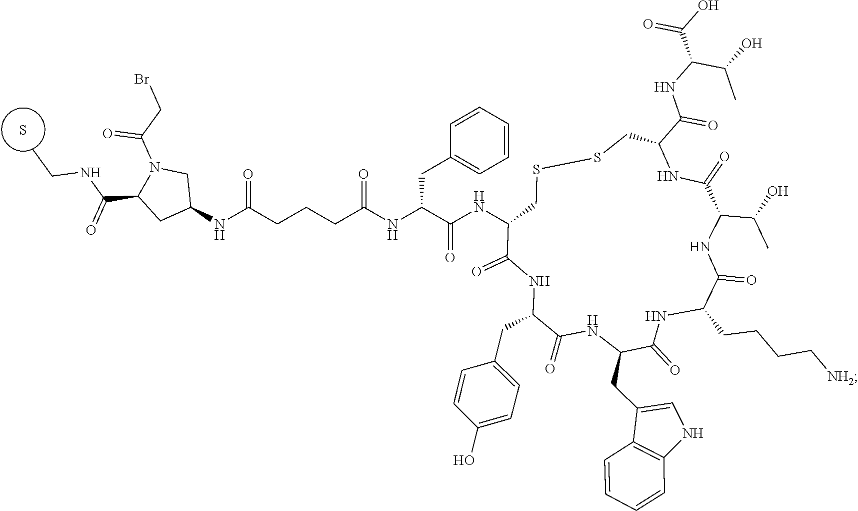

and compound (A) is

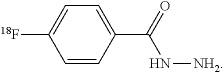 [SMILES: NNC(=O)c1ccc([18F])cc1]

S is a solid support as described herein, including in embodiments.

In embodiments, compound (I^{B}) is

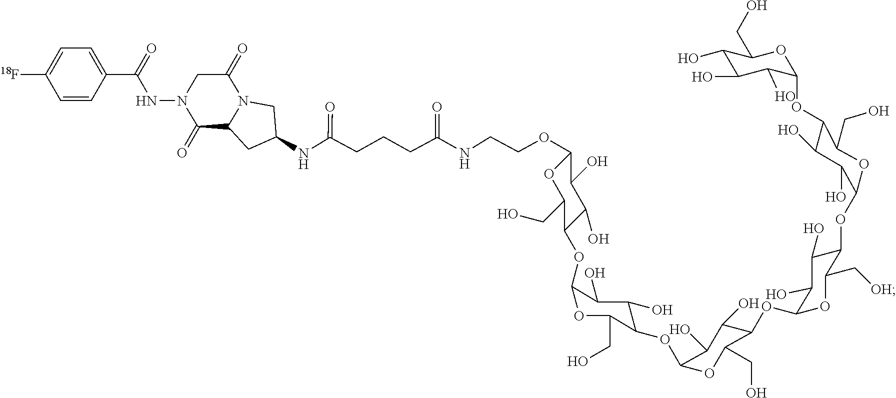

compound (IV^{B}) is

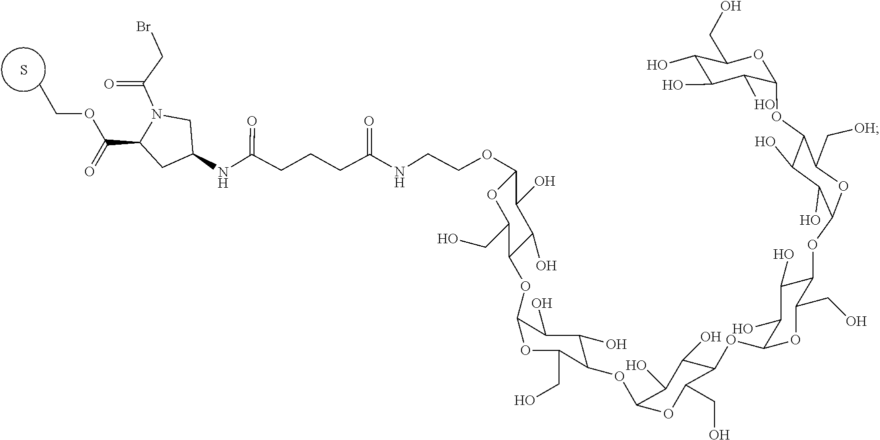

and compound (A) is

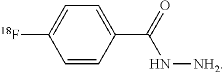 [SMILES: NNC(=O)c1ccc([18F])cc1]

S is a solid support as described herein, including in embodiments.

In embodiments, compound (I^{B}) is

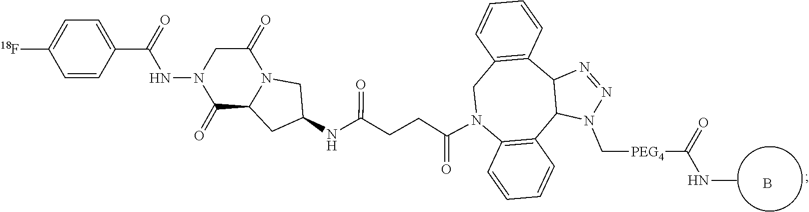

compound (IV^{B}) is

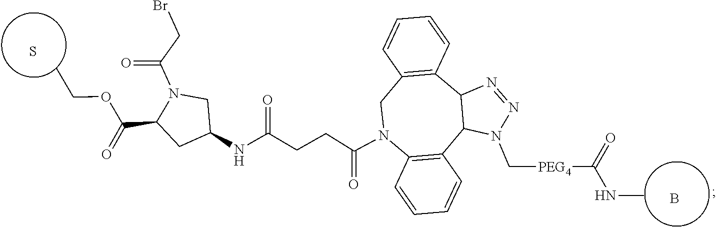

and compound (A) is

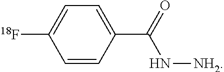 [SMILES: NNC(=O)c1ccc([18F])cc1]

S is a solid support as described herein, including in embodiments. B is a monovalent form of a Her2 receptor binding nanobody.

In embodiments, compound (I^{B}) is

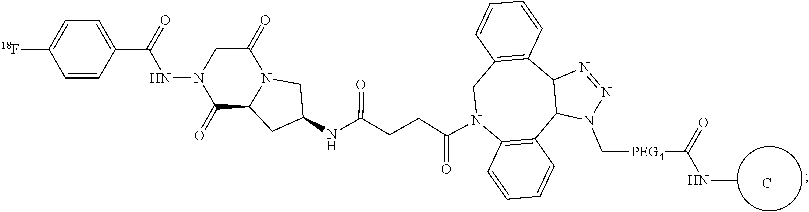

compound (IV^{B}) is

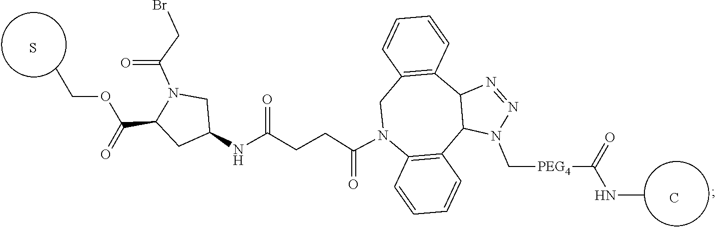

and compound (A) is

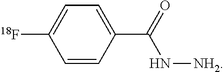 [SMILES: NNC(=O)c1ccc([18F])cc1]

S is a solid support as described herein, including in embodiments. C is a monovalent form of a TNF-α binding antibody.

In embodiments, the method of making compound (I^{B}) includes mixing compound (IV^{B}) and compound (A) in a reaction vessel. In embodiments, the method of making compound (I^{B}) includes mixing compound (V^{B}) and compound (A) in a reaction vessel.

In embodiments, the method further includes mixing compound (IV^{B}) or compound (V^{B}) and compound (A) in a basic solution. In embodiments, the method further includes mixing compound (IV^{B}) and compound (A) in a basic solution. In embodiments, the method further includes mixing compound (V^{B}) and compound (A) in a basic solution. In embodiments, the basic solution has a pH from about 7.1 to about 8. In embodiments, the basic solution has a pH from about 7.1 to about 7.8. In embodiments, the basic solution has a pH from about 7.1 to about 7.6. In embodiments, the basic solution has a pH from about 7.2 to about 7.5. In embodiments, the basic solution has a pH from about 7.3 to about 7.5. In embodiments, the basic solution has a pH of about 7.3. In embodiments, the basic solution has a pH of about 7.4. In embodiments, the basic solution has a pH of about 7.5. In embodiments, the basic solution has a pH from 7.1 to 8. In embodiments, the basic solution has a pH from 7.1 to 7.8. In embodiments, the basic solution has a pH from 7.1 to 7.6. In embodiments, the basic solution has a pH from 7.2 to 7.5. In embodiments, the basic solution has a pH from 7.3 to 7.5. In embodiments, the basic solution has a pH of 7.3. In embodiments, the basic solution has a pH of 7.4. In embodiments, the basic solution has a pH of 7.5.

In an aspect is provided a method of making compound (I^{C}), or a pharmaceutically acceptable salt thereof, the method including mixing compound (II^{C}) or compound (III^{C}) and compound (A) in a reaction vessel; wherein compound (I^{C}) has the formula:

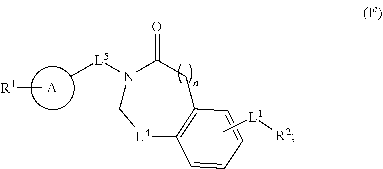

compound (II^{C}) has the formula:

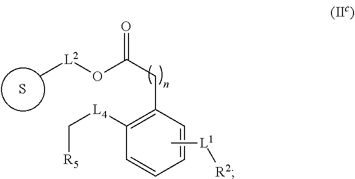

compound (III^{C}) has the formula:

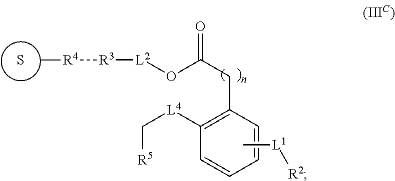

and compound (A) has the formula:

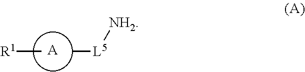

Ring A, S, L¹, L², L⁴, L⁵, R¹, R², R³, R⁴, R⁵, and n are as described herein, including in embodiments. The symbol ---- is a noncovalent or covalent bond.

In embodiments, compound (I^{C}) has the formula:

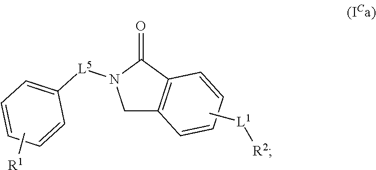

compound (II^{C}) has the formula:

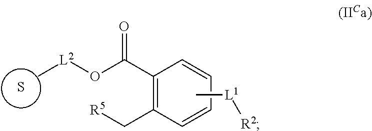

and compound (III^{C}) has the formula:

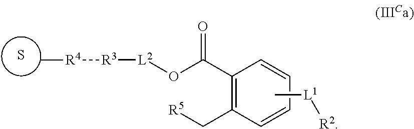

S, L¹, L², L⁵, R¹, R², R³, R⁴, and R⁵ are as described herein, including in embodiments.

In embodiments, compound (I^{C}) has the formula:

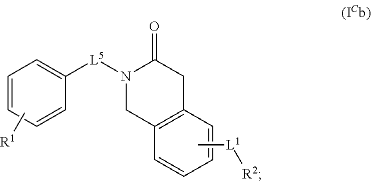

compound (II^{C}) has the formula:

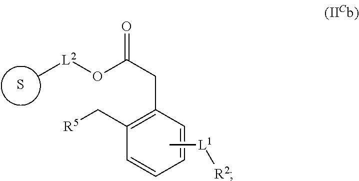

and compound (III^{C}) has the formula:

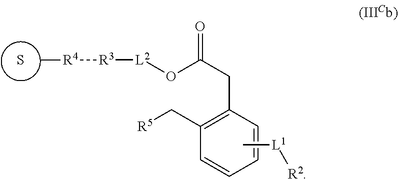

S, L¹, L², L⁵, R¹, R², R³, R⁴, and R⁵ are as described herein, including in embodiments.

In embodiments, compound (I^{C}) is

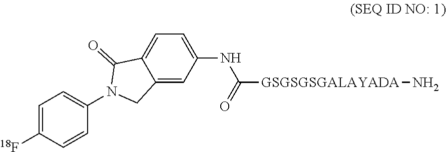

and compound (II^{C}) is

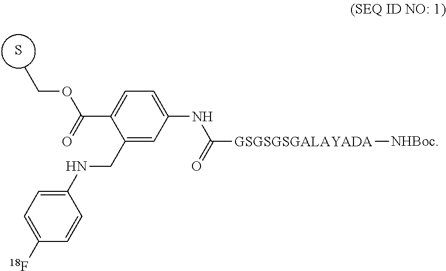

S is a solid support as described herein, including in embodiments.

In embodiments, compound (I^{C}) is

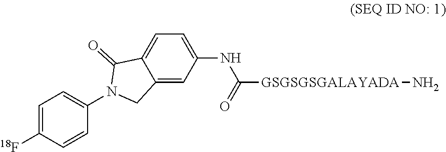

and compound (III^{C}) is

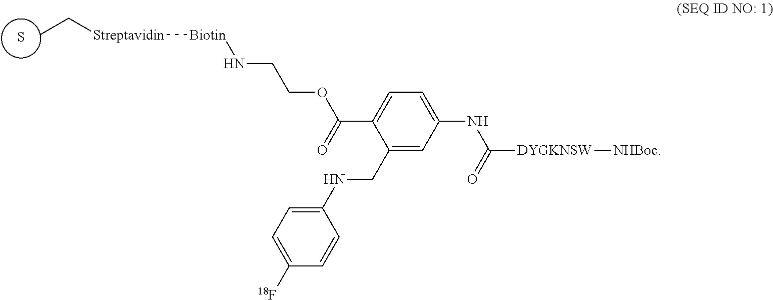

S is a solid support as described herein, including in embodiments.

In embodiments, compound (I^{C}) is

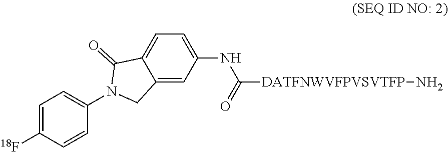

and compound (II^{C}) is

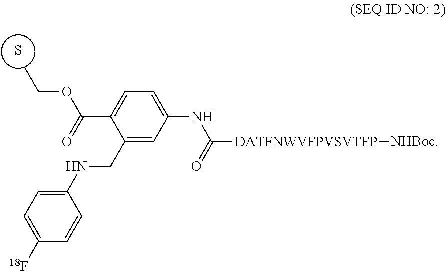

S is a solid support as described herein, including in embodiments.

In embodiments, compound (I^{C}) is

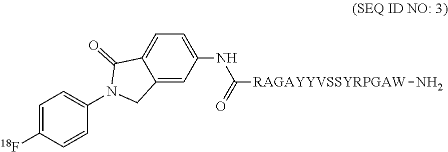

and compound (II^{C}) is

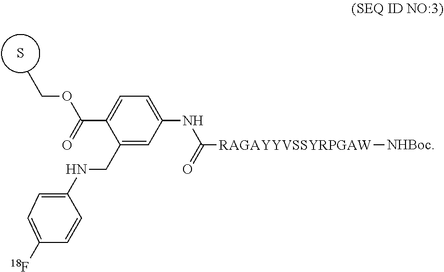

S is a solid support as described herein, including in embodiments.

In embodiments, compound (I^{C}) is

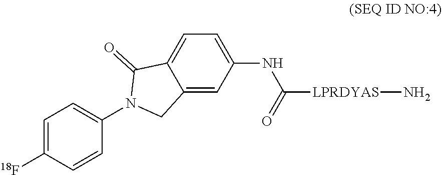

and compound (II^{C}) is

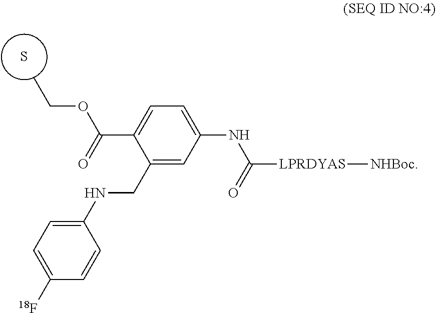

S is a solid support as described herein, including in embodiments.

In embodiments, compound (I^{C}) is

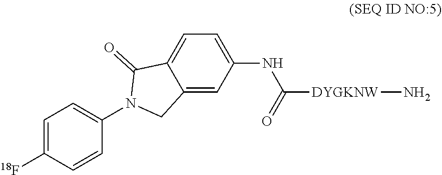

and compound (II^{C}) is

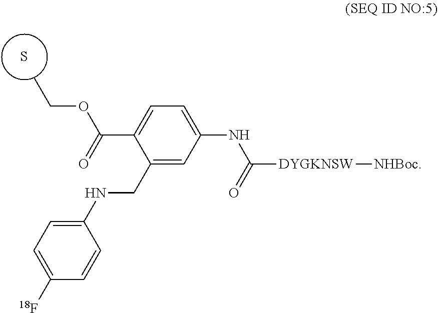

S is a solid support as described herein, including in embodiments.

In embodiments, the method of making compound (I^{C}) includes mixing compound (II^{C}) and compound (A) in a reaction vessel. In embodiments, the method of making compound (I^{C}) includes mixing compound (III^{C}) and compound (A) in a reaction vessel.

In embodiments, the method further includes mixing compound (II^{C}) or compound (III^{C}) and compound (A) in a basic solution. In embodiments, the method further includes mixing compound (II^{C}) and compound (A) in a basic solution. In embodiments, the method further includes mixing compound (III^{C}) and compound (A) in a basic solution. In embodiments, the basic solution has a pH from about 7.1 to about 8. In embodiments, the basic solution has a pH from about 7.1 to about 7.8. In embodiments, the basic solution has a pH from about 7.1 to about 7.6. In embodiments, the basic solution has a pH from about 7.2 to about 7.5. In embodiments, the basic solution has a pH from about 7.3 to about 7.5. In embodiments, the basic solution has a pH of about 7.3. In embodiments, the basic solution has a pH of about 7.4. In embodiments, the basic solution has a pH of about 7.5. In embodiments, the basic solution has a pH from 7.1 to 8. In embodiments, the basic solution has a pH from 7.1 to 7.8. In embodiments, the basic solution has a pH from 7.1 to 7.6. In embodiments, the basic solution has a pH from 7.2 to 7.5. In embodiments, the basic solution has a pH from 7.3 to 7.5. In embodiments, the basic solution has a pH of 7.3. In embodiments, the basic solution has a pH of 7.4. In embodiments, the basic solution has a pH of 7.5.

In an aspect is provided a method of making compound (I^{D}), or a pharmaceutically acceptable salt thereof, the method including mixing compound (II^{D}) or compound (III^{D}) and compound (A) in a reaction vessel; wherein compound (I^{D}) ha the fora:

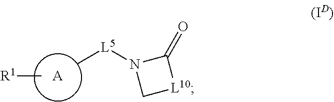

compound (II^{D}) has the formula:

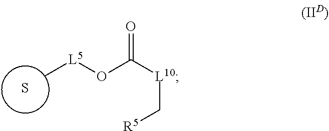

compound (III^{D}) has the formula:

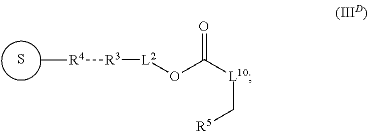

and compound (A) has the formula:

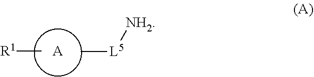

Ring A, S, L, R¹, R², R³, R⁴, and R⁵ are as described herein, including in embodiments. The symbol ---- is a noncovalent or covalent bond.

L¹⁰ is —N(R¹⁰⁰)—, —S—, —S(O)₂—, —O—, —C(O)—, —C(O)O—, —OC(O)—, —N(R¹⁰⁰)C(O)—, —C(O)N(R¹⁰⁰)—, —NR¹⁰⁰C(O)NR¹⁰⁰—, —NR¹⁰⁰C(NH)NR¹⁰⁰—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered).

R¹⁰⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, compound (II^{D}) and compound (A) react to form a radiolabeled product (i.e., compound (I^{D})). In embodiments, compound (III^{D}) and compound (A) react to form a radiolabeled product (i.e., compound (I^{D})). In embodiments of formula (I^{D}), R′ is a radioisotope.

In embodiments, a substituted L¹⁰ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰ is substituted with a plurality of groups selected from substituent groups, size-limited substituent groups, and lower substituent groups; each substituent group, size-limited substituent group, and/or lower substituent group may optionally be different. In embodiments, when L¹⁰ is substituted, it is substituted with at least one substituent group. In embodiments, when L¹⁰ is substituted, it is substituted with at least one size-limited substituent group. In embodiments, when L¹⁰ is substituted, it is substituted with at least one lower substituent group.

In embodiments, L¹⁰ is substituted with a monovalent form or a drug or a monovalent form of a biomolecule.

In embodiments, a substituted R¹⁰⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰⁰ is substituted with a plurality of groups selected from substituent groups, size-limited substituent groups, and lower substituent groups; each substituent group, size-limited substituent group, and/or lower substituent group may optionally be different. In embodiments, when R¹⁰⁰ is substituted, it is substituted with at least one substituent group. In embodiments, when R¹⁰⁰ is substituted, it is substituted with at least one size-limited substituent group. In embodiments, when R¹⁰⁰ is substituted, it is substituted with at least one lower substituent group.

In embodiments, R¹⁰⁰ is independently hydrogen or substituted or unsubstituted alkyl. In embodiments, R¹⁰⁰ is independently hydrogen or substituted or unsubstituted C₁-C₄ alkyl. In embodiments, R¹⁰⁰ is independently hydrogen. In embodiments, R¹⁰⁰ is independently unsubstituted C₁-C₄ alkyl. In embodiments, R¹⁰⁰ is independently unsubstituted methyl. In embodiments, R¹⁰⁰ is independently unsubstituted ethyl. In embodiments, R¹⁰⁰ is independently unsubstituted propyl. In embodiments, R¹⁰⁰ is independently unsubstituted n-propyl. In embodiments, R¹⁰⁰ is independently unsubstituted isopropyl. In embodiments, R¹⁰⁰ is independently unsubstituted butyl. In embodiments, R¹⁰⁰ is independently unsubstituted n-butyl. In embodiments, R¹⁰⁰ is independently unsubstituted tert-butyl.

In embodiments, compound (A) is

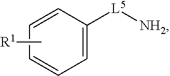

wherein L⁵ and R¹ are as described herein, including in embodiments. In embodiments, compound (A) is

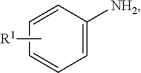

wherein R¹ is as described herein, including in embodiments. In embodiments, compound (A) is

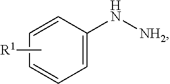

wherein R¹ is as described herein, including in embodiments. In embodiments, compound (A) is

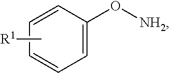

wherein R¹ is as described herein, including in embodiments. In embodiments, compound (A) is

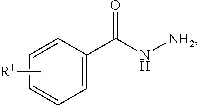

wherein R¹ is as described herein, including in embodiments. In embodiments, compound (A) is

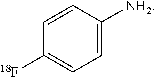

In embodiments, compound (A) is

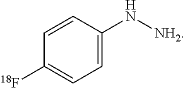

In embodiments, compound (A) is

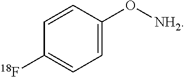

In embodiments, compound (A) is

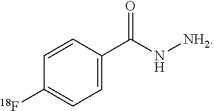 [SMILES: NNC(=O)c1ccc([18F])cc1]

### VI. Embodiments

Embodiment P1. A compound, or a pharmaceutically acceptable salt thereof, having the formula:

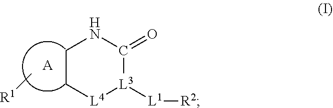

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R_{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4; and
m2 and v2 are each independently 1 or 2.

Embodiment P2. The compound of embodiment P1, wherein L³ is an unsubstituted heterocycloalkylene.

Embodiment P3. The compound of one of embodiments P1 to P2, wherein Ring A is a C₃-C₆ cycloalkylene, 3 to 6 membered heterocycloalkylene, or phenylene.

Embodiment P4. The compound of one of embodiments P1 to P2, wherein Ring A is phenylene.

Embodiment P5. The compound of embodiment P1, having the formula:

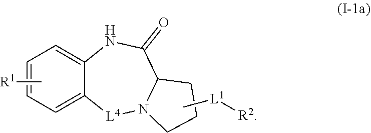

Embodiment P6. The compound of one of embodiments P1 to P5, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment P7. The compound of one of embodiments P1 to P5, wherein L¹ is —NHC(O)—.

Embodiment P8. The compound of one of embodiments P1 to P7, wherein L⁴ is —C(O)— or unsubstituted methylene.

Embodiment P9. The compound of one of embodiments P1 to P7, wherein L⁴ is —C(O)—.

Embodiment P10. The compound of one of embodiments P1 to P9, wherein R¹ is —¹⁸F.

Embodiment P11. The compound of one of embodiments P1 to P10, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P12. The compound of one of embodiments P1 to P10, wherein R² is a monovalent form of a drug or substituted heteroalkyl.

Embodiment P13. The compound of one of embodiments P1 to P10, wherein R² is a monovalent form of a drug.

Embodiment P14. A pharmaceutical composition comprising a compound of one of embodiments P1 to P13, or a pharmaceutically acceptable salt thereof, and a pharmaceutically acceptable excipient.

Embodiment P15. A method of detecting a level of a compound in a subject, the method comprising:
(i) administering to the subject a compound of one of embodiments P1 to P13, or a pharmaceutically acceptable salt thereof; and
(ii) detecting the level of the compound in the subject.

Embodiment P16. The method of embodiment P15, wherein step (ii) further comprises detecting the level of the compound in the subject using positron emission tomography (PET), wherein R¹ is a PET detectable radioisotope.

Embodiment P17. The method of embodiment P16, further comprising detecting a physiological location of the compound in the subject using PET.

Embodiment P18. A method of detecting the level of CD44v6 in a subject, the method comprising administering to the subject an effective amount of a compound of one of embodiments P1 to P13, or a pharmaceutically acceptable salt thereof.

Embodiment P19. The method of embodiment P18, further comprising detecting the level of the compound using positron emission tomography.

Embodiment P20. A compound, or a pharmaceutically acceptable salt thereof, having the formula:

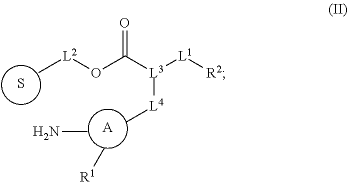

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
S is a solid support.

Embodiment P21. The compound of embodiment P20, wherein L³ is an unsubstituted heterocycloalkylene.

Embodiment P22. The compound of one of embodiments P20 to P21, wherein Ring A is a C₃-C₆ cycloalkylene, 3 to 6 membered heterocycloalkylene, or phenylene.

Embodiment P23. The compound of one of embodiments P20 to P21, wherein Ring A is phenylene.

Embodiment P24. The compound of embodiment P20, having the formula:

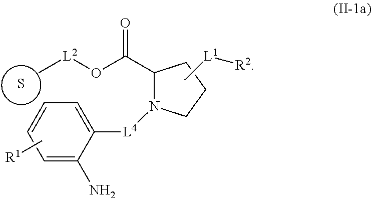

Embodiment P25. A compound, or a pharmaceutically acceptable salt thereof, having the formula:

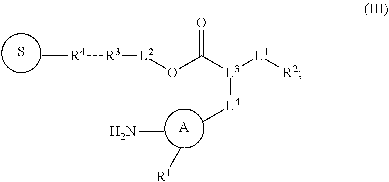

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment P26. The compound of embodiment P25, wherein L³ is an unsubstituted heterocycloalkylene.

Embodiment P27. The compound of one of embodiments P25 to P26, wherein Ring A is a C₃-C₆ cycloalkylene, 3 to 6 membered heterocycloalkylene, or phenylene.

Embodiment P28. The compound of one of embodiments P25 to P26, wherein Ring A is phenylene.

Embodiment P29. The compound of embodiment P25, having the formula:

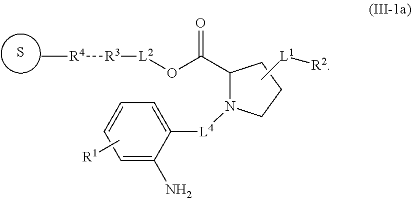

Embodiment P30. The compound of one of embodiments P25 to P29, wherein ---- is a noncovalent bond.

Embodiment P31. The compound of one of embodiments P20 to P30, wherein S is a chromatographic material.

Embodiment P32. The compound of one of embodiments P20 to P31, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment P33. The compound of one of embodiments P20 to P31, wherein L¹ is —NHC(O)—.

Embodiment P34. The compound of one of embodiments P20 to P33, wherein L² is a substituted or unsubstituted alkylene or substituted or unsubstituted heteroalkylene.

Embodiment P35. The compound of one of embodiments P20 to P33, wherein L² is an unsubstituted C₁-C₄ alkylene.

Embodiment P36. The compound of one of embodiments P20 to P33, wherein L² is an unsubstituted methylene.

Embodiment P37. The compound of one of embodiments P20 to P36, wherein L⁴ is —C(O)— or unsubstituted methylene.

Embodiment P38. The compound of one of embodiments P20 to P36, wherein L⁴ is —C(O)—.

Embodiment P39. The compound of one of embodiments P20 to P36, wherein R¹ is —¹⁸F.

Embodiment P40. The compound of one of embodiments P20 to P39, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P41. The compound of one of embodiments P20 to P39, wherein R² is a monovalent form of a drug or substituted heteroalkyl.

Embodiment P42. The compound of one of embodiments P20 to P39, wherein R² is a monovalent form of a drug.

Embodiment P43. A method of making compound (I), or a pharmaceutically acceptable salt thereof, said method comprising mixing compound (II) or compound (III) in a reaction vessel; wherein
compound (I) has the formula:

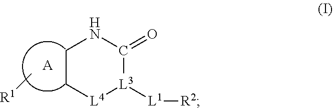

compound (II) has the formula:

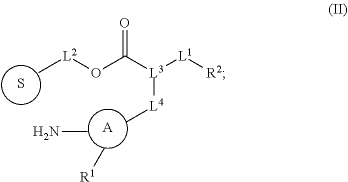

compound (III) has the formula:

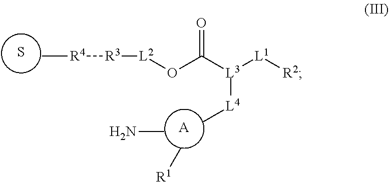

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment P44. The method of embodiment P43, further comprising mixing compound (II) or compound (III) in a basic solution.

Embodiment P45. The method of embodiment P44, wherein the basic solution has a pH from 7.1 to 8.

Embodiment P46. The method of embodiment P45, wherein the basic solution has a pH of 7.4.

Embodiment P47. The method of one of embodiments P43 to P46, wherein L³ is an unsubstituted heterocycloalkylene.

Embodiment P48. The method of one of embodiments P43 to P47, wherein Ring A is a C₃-C₆ cycloalkylene, 3 to 6 membered heterocycloalkylene, or phenylene.

Embodiment P49. The method of one of embodiments P43 to P47, wherein Ring A is phenylene.

Embodiment P50. The method of one of embodiments P43 to P46, wherein
compound (I) has the formula:

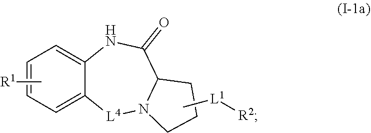

compound (II) has the formula:

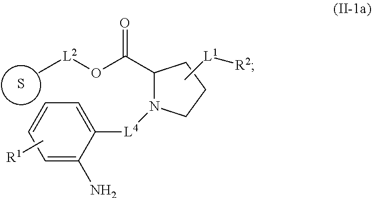

and
compound (III) has the formula:

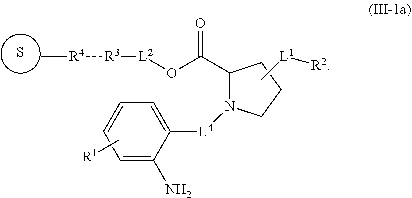

Embodiment P51. The method of one of embodiments P43 to P50, wherein ---- is a noncovalent bond.

Embodiment P52. The method of one of embodiments P43 to P51, wherein S is a chromatographic material.

Embodiment P53. The method of one of embodiments P43 to P52, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment P54. The method of one of embodiments P43 to P52, wherein L¹ is —NHC(O)—.

Embodiment P55. The method of one of embodiments P43 to P54, wherein L² is a substituted or unsubstituted alkylene or substituted or unsubstituted heteroalkylene.

Embodiment P56. The method of one of embodiments P43 to P54, wherein L² is an unsubstituted C₁-C₄ alkylene.

Embodiment P57. The method of one of embodiments P43 to P54, wherein L² is an unsubstituted methylene.

Embodiment P58. The method of one of embodiments P43 to P57, wherein L⁴ is —C(O)— or unsubstituted methylene.

Embodiment P59. The method of one of embodiments P43 to P57, wherein L⁴ is —C(O)—.

Embodiment P60. The method of one of embodiments P43 to P59, wherein R¹ is —¹⁸F.

Embodiment P61. The method of one of embodiments P43 to P60, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P62. The method of one of embodiments P43 to P60, wherein R² is a monovalent form of a drug or substituted heteroalkyl.

Embodiment P63. The method of one of embodiments P43 to P60, wherein R² is a monovalent form of a drug.

Embodiment Q1. A compound, or a pharmaceutically acceptable salt thereof, having the formula:

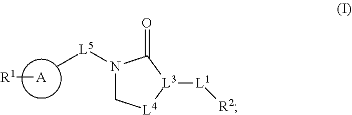

wherein
Ring A is a C₃-C₆ cycloalkylene, 3 to 6 membered heterocycloalkylene, phenylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene; R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4; and
m2 and v2 are each independently 1 or 2.

Embodiment Q2. The compound of embodiment Q1, wherein L³ is an unsubstituted heterocycloalkylene.

Embodiment Q3. The compound of embodiment Q1, having the formula:

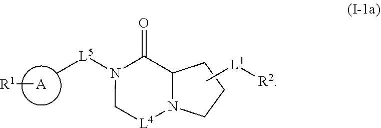

Embodiment Q4. The compound of one of embodiments Q1 to Q3, wherein Ring A is a C₃-C₆ cycloalkylene, 3 to 6 membered heterocycloalkylene, or phenylene.

Embodiment Q5. The compound of one of embodiments Q1 to Q3, wherein Ring A is phenylene.

Embodiment Q6. The compound of one of embodiments Q1 to Q5, wherein L⁴ is —C(O)—.

Embodiment Q7. The compound of one of embodiments Q1 to Q6, wherein L⁵ is a bond, —NH—, —O—, or —C(O)NH—.

Embodiment Q8. The compound of one of embodiments Q1 to Q7, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment Q9. The compound of one of embodiments Q1 to Q7, wherein L¹ is —NHC(O)—.

Embodiment Q10. The compound of one of embodiments Q1 to Q9, wherein R¹ is -¹⁸F.

Embodiment Q11. The compound of one of embodiments Q1 to Q10, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment Q12. The compound of one of embodiments Q1 to Q10, wherein R² is a monovalent form of a drug or substituted heteroalkyl.

Embodiment Q13. The compound of one of embodiments Q1 to Q10, wherein R² is a monovalent form of a drug.

Embodiment Q14. A pharmaceutical composition comprising a compound of one of embodiments Q1 to Q13, or a pharmaceutically acceptable salt thereof, and a pharmaceutically acceptable excipient.

Embodiment Q15. A method of detecting a level of a compound in a subject, the method comprising:
(i) administering to the subject a compound of one of embodiments Q1 to Q13, or a pharmaceutically acceptable salt thereof, and
(ii) detecting the level of the compound in the subject.

Embodiment Q16. The method of embodiment Q15, wherein step (ii) further comprises detecting the level of the compound in the subject using positron emission tomography (PET), wherein R¹ is a PET detectable radioisotope.

Embodiment Q17. The method of embodiment Q16, further comprising detecting a physiological location of the compound in the subject using PET.

Embodiment Q18. A method of detecting the level of CD44v6 in a subject, the method comprising administering to the subject an effective amount of a compound of one of embodiments Q1 to Q13, or a pharmaceutically acceptable salt thereof.

Embodiment Q19. The method of embodiment Q18, further comprising detecting the level of the compound using positron emission tomography.

Embodiment Q20. A compound, or a pharmaceutically acceptable salt thereof, having the formula:

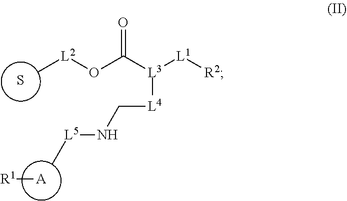

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
S is a solid support.

Embodiment Q21. The compound of embodiment Q20, having the formula:

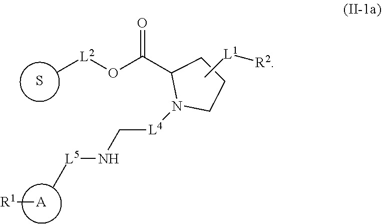

Embodiment Q22. A compound, or a pharmaceutically acceptable salt thereof, having the formula:

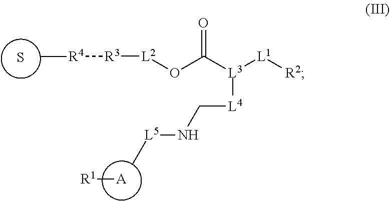

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment Q23. The compound of embodiment Q22, having the formula:

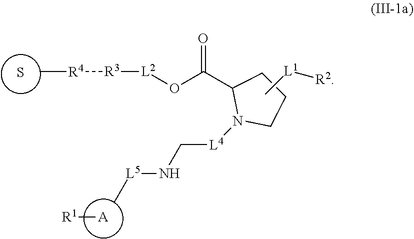

Embodiment Q24. The compound of one of embodiments Q22 to Q23, wherein ---- is a noncovalent bond.

Embodiment Q25. The compound of one of embodiments Q20 to Q24, wherein Ring A is a C₃-C₆ cycloalkylene, 3 to 6 membered heterocycloalkylene, or phenylene.

Embodiment Q26. The compound of one of embodiments Q20 to Q24, wherein Ring A is phenylene.

Embodiment Q27. The compound of one of embodiments Q20 to Q26, wherein R¹ is —¹⁸F.

Embodiment Q28. A compound, or a pharmaceutically acceptable salt thereof, having the formula:

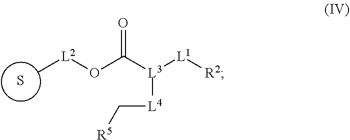

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R⁵ is a leaving group; and
S is a solid support.

Embodiment Q29. The compound of embodiment Q28, having the formula:

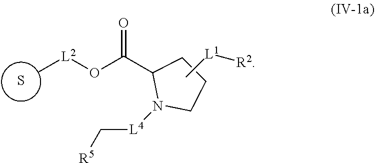

Embodiment Q30. A compound, or a pharmaceutically acceptable salt thereof, having the formula:

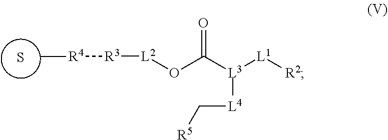

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
---- is a noncovalent or covalent bond; and
S is a solid support.

Embodiment Q31. The compound of embodiment Q30, having the formula:

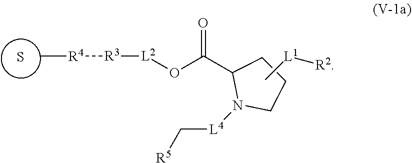

Embodiment Q32. The compound of one of embodiments Q30 to Q31, wherein ---- is a noncovalent bond.

Embodiment Q33. The compound of one of embodiments Q28 to Q32, wherein R⁵ is a halogen,

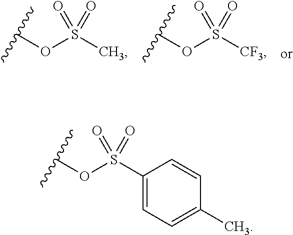 [SMILES: *OS(C)(=O)=O ; *OS(=O)(=O)C(F)(F)F ; *OS(=O)(=O)c1ccc(C)cc1]

Embodiment Q34. The compound of one of embodiments Q28 to Q32, wherein R⁵ is —Br.

Embodiment Q35. The compound of one of embodiments Q20 to Q34, wherein S is a chromatographic material.

Embodiment Q36. The compound of one of embodiments Q20 to Q35, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment Q37. The compound of one of embodiments Q20 to Q35, wherein L¹ is —NHC(O)—.

Embodiment Q38. The compound of one of embodiments Q20 to Q37, wherein L² is a substituted or unsubstituted alkylene or substituted or unsubstituted heteroalkylene.

Embodiment Q39. The compound of one of embodiments Q20 to Q37, wherein L² is an unsubstituted C₁-C₄ alkylene.

Embodiment Q40. The compound of one of embodiments Q20 to Q37, wherein L² is an unsubstituted methylene.

Embodiment Q41. The compound of one of embodiments Q20 to Q40, wherein L⁴ is —C(O)—.

Embodiment Q42. The compound of one of embodiments Q20 to Q41, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment Q43. The compound of one of embodiments Q20 to Q41, wherein R² is a monovalent form of a drug or substituted heteroalkyl.

Embodiment Q44. The compound of one of embodiments Q20 to Q41, wherein R² is a monovalent form of a drug.

Embodiment Q45. A method of making compound (I), or a pharmaceutically acceptable salt thereof, said method comprising mixing compound (II) or compound (III) in a reaction vessel; wherein
compound (I) has the formula: R (I);

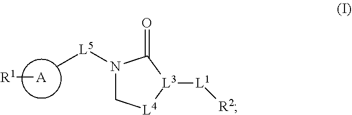

compound (II) has the formula: (II);

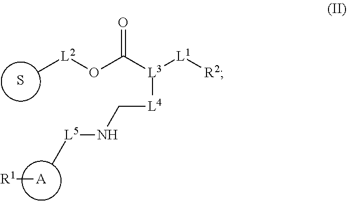

compound (III) has the formula: (III);

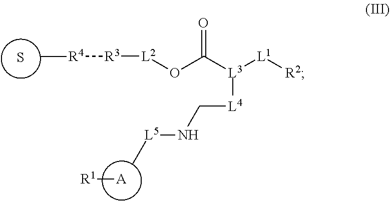

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment Q46. The method of embodiment Q45, wherein
compound (I) has the formula:

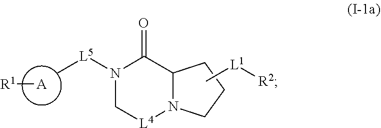

compound (II) has the formula:

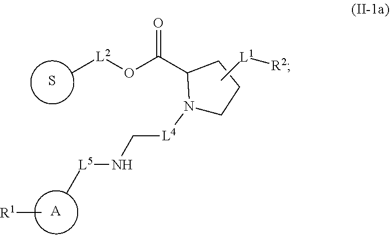

and
compound (III) has the formula:

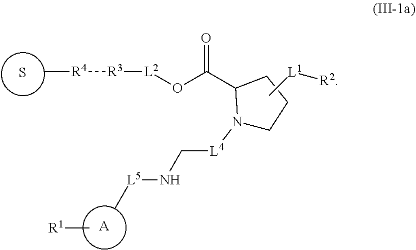

Embodiment Q47. The method of one of embodiments Q45 to Q46, further comprising mixing compound (II) or compound (III) in a basic solution.

Embodiment Q48. The method of embodiment Q47, wherein the basic solution has a pH from 7.1 to 8.

Embodiment Q49. The method of embodiment Q48, wherein the basic solution has a pH of 7.4

Embodiment Q50. A method of making compound (I), or a pharmaceutically acceptable salt thereof, said method comprising mixing compound (IV) or compound (V) and compound (A) together in a reaction vessel; wherein
compound (I) has the formula:

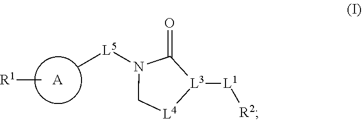

compound (IV) has the formula:

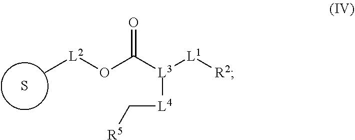

compound (V) has the formula:

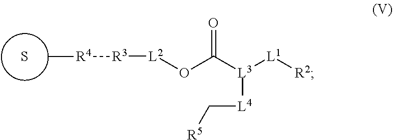

compound (A) has the formula:

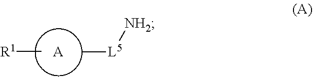

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene; R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
---- is a noncovalent or covalent bond; and
S is a solid support.

Embodiment Q51. The method of embodiment Q50, wherein
compound (I) has the formula:

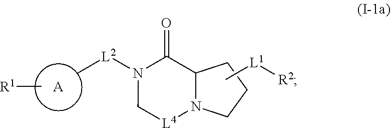

compound (IV) has the formula:

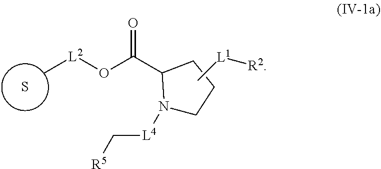

and
compound (V) has the formula:

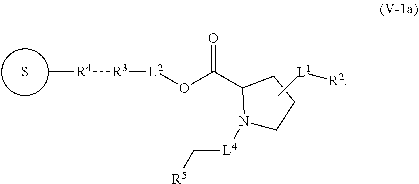

Embodiment Q52. The method of one of embodiments Q50 to Q51, wherein R⁵ is a halogen,

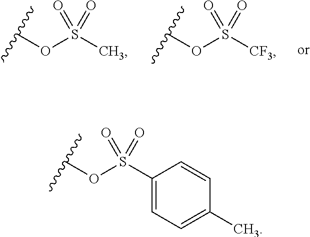 [SMILES: *OS(C)(=O)=O ; *OS(=O)(=O)C(F)(F)F ; "or" ; *OS(=O)(=O)c1ccc(C)cc1]

Embodiment Q53. The method of one of embodiments Q50 to Q51, wherein R⁵ is —Br.

Embodiment Q54. The method of one of embodiments Q45 to Q53, wherein ---- is a noncovalent bond.

Embodiment Q55. The method of one of embodiments Q45 to Q54, wherein S is a chromatographic material.

Embodiment Q56. The method of one of embodiments Q45 to Q55, wherein Ring A is a C₃-C₆ cycloalkylene, 3 to 6 membered heterocycloalkylene, or phenylene.

Embodiment Q57. The method of one of embodiments Q45 to Q55, wherein Ring A is phenylene.

Embodiment Q58. The method of one of embodiments Q45 to Q57, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment Q59. The method of one of embodiments Q45 to Q57, wherein L¹ is —NHC(O)—.

Embodiment Q60. The method of one of embodiments Q45 to Q59, wherein L² is a substituted or unsubstituted alkylene or substituted or unsubstituted heteroalkylene.

Embodiment Q61. The method of one of embodiments Q45 to Q59, wherein L² is an unsubstituted C₁-C₄ alkylene.

Embodiment Q62. The method of one of embodiments Q45 to Q59, wherein L² is an unsubstituted methylene.

Embodiment Q63. The method of one of embodiments Q45 to Q62, wherein L⁴ is —C(O)—.

Embodiment Q64. The method of one of embodiments Q45 to Q63, wherein L⁵ is a bond, —NH—, —O—, or —C(O)NH—.

Embodiment Q65. The method of one of embodiments Q45 to Q64, wherein R¹ is -¹⁸F.

Embodiment Q66. The method of one of embodiments Q45 to Q65, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment Q67. The method of one of embodiments Q45 to Q65, wherein R² is a monovalent form of a drug or substituted heteroalkyl.

Embodiment Q68. The method of one of embodiments Q45 to Q65, wherein R² is a monovalent form of a drug.

Embodiment S1. A compound, or a pharmaceutically acceptable salt thereof, having the formula:

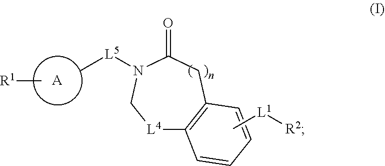

wherein
Ring A is a C₃-C₆ cycloalkylene, 3 to 6 membered heterocycloalkylene, phenylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
n is 0 or 1.

Embodiment S2. The compound of embodiment S1, wherein Ring A is a C₃-C₆ cycloalkylene, 3 to 6 membered heterocycloalkylene, or phenylene.

Embodiment S3. The compound of embodiment S1, wherein Ring A is phenylene.

Embodiment S4. The compound of one of embodiments S1 to S3, wherein L⁴ is a bond.

Embodiment S5. The compound of one of embodiments S1 to S4, wherein L⁵ is a bond.

Embodiment S6. The compound of one of embodiments S1 to S5, wherein n is 0.

Embodiment S7. The compound of one of embodiments S1 to S5, wherein n is 1.

Embodiment S8. The compound of embodiment S1, having the formula:

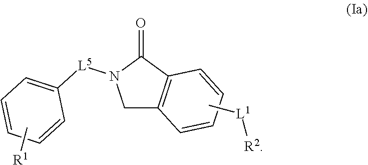

Embodiment S9. The compound of embodiment S1, having the formula:

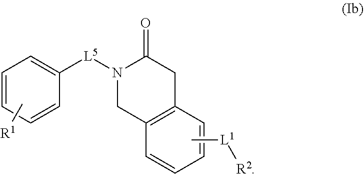

Embodiment S10. The compound of one of embodiments S1 to S9, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment S11. The compound of one of embodiments S1 to S9, wherein L¹ is —NHC(O)—.

Embodiment S12. The compound of one of embodiments S1 to S11, wherein R¹ is —¹⁸F.

Embodiment S13. The compound of one of embodiments S1 to S12, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment S14. The compound of one of embodiments S1 to S12, wherein R² is a monovalent form of a drug or substituted heteroalkyl.

Embodiment S15. The compound of one of embodiments S1 to S12, wherein R² is a monovalent form of a drug.

Embodiment S16. A pharmaceutical composition comprising a compound of one of embodiments S1 to S15, or a pharmaceutically acceptable salt thereof, and a pharmaceutically acceptable excipient.

Embodiment S17. A method of detecting the level of a compound in a subject, the method comprising:
(i) administering to the subject a compound of one of embodiments S1 to S15, or a pharmaceutically acceptable salt thereof, and
(ii) detecting the level of the compound in the subject.

Embodiment S18. The method of embodiment S17, wherein step (ii) further comprises detecting the level of the compound in the subject using positron emission tomography (PET), wherein R¹ is a PET detectable radioisotope.

Embodiment S19. The method of embodiment S18, further comprising detecting a physiological location of the compound in the subject using PET.

Embodiment S20. A method of detecting the level of CD44v6 in a subject, the method comprising administering to the subject an effective amount of a compound of one of embodiments S1 to S15, or a pharmaceutically acceptable salt thereof.

Embodiment S21. The method of embodiment S20, further comprising detecting the level of the compound using positron emission tomography.

Embodiment S22. A compound, or a pharmaceutically acceptable salt thereof, having the formula:

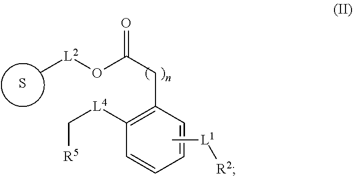

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R⁵ is a leaving group;
n is 0 or 1; and
S is a solid support.

Embodiment S23. A compound, or a pharmaceutically acceptable salt thereof, having the formula:

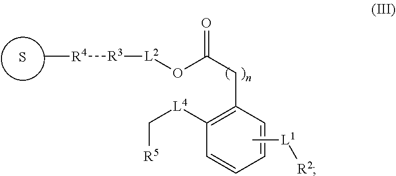

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰-, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
n is 0 or 1;
---- is a noncovalent or covalent bond; and
S is a solid support.

Embodiment S24. The compound of embodiment S23, wherein ---- is a noncovalent bond.

Embodiment S25. The compound of one of embodiments S22 to S24, wherein S is a chromatographic material.

Embodiment S26. The compound of one of embodiments S22 to S25, wherein n is 0.

Embodiment S27. The compound of one of embodiments S22 to S25, wherein n is 1.

Embodiment S28. The compound of one of embodiments S22 to S27, wherein R⁵ is a halogen,

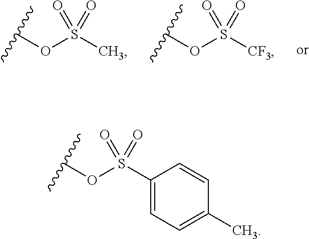 [SMILES: *OS(C)(=O)=O ; *OS(=O)(=O)C(F)(F)F ; "or" ; *OS(=O)(=O)c1ccc(C)cc1]

Embodiment S29. The compound of one of embodiments S22 to S27, wherein R⁵ is —Br.

Embodiment S30. The compound of one of embodiments S22 to S29, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment S31. The compound of one of embodiments S22 to S29, wherein L¹ is —NHC(O)—.

Embodiment S32. The compound of one of embodiments S22 to S31, wherein L² is a substituted or unsubstituted alkylene or substituted or unsubstituted heteroalkylene.

Embodiment S33. The compound of one of embodiments S22 to S31, wherein L² is an unsubstituted C₁-C₄ alkylene.

Embodiment S34. The compound of one of embodiments S22 to S31, wherein L² is an unsubstituted methylene.

Embodiment S35. The compound of one of embodiments S22 to S34, wherein L⁴ is a bond.

Embodiment S36. The compound of one of embodiments S22 to S35, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment S37. The compound of one of embodiments S22 to S35, wherein R² is a monovalent form of a drug or substituted heteroalkyl.

Embodiment S38. The compound of one of embodiments S22 to S35, wherein R² is a monovalent form of a drug.

Embodiment S39. A method of making compound (I), or a pharmaceutically acceptable salt thereof, said method comprising mixing compound (II) or compound (III) and compound (A) together in a reaction vessel; wherein
compound (I) has the formula:

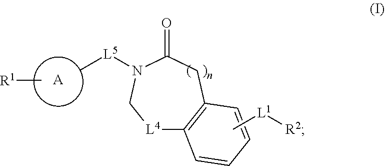

compound (II) has the formula:

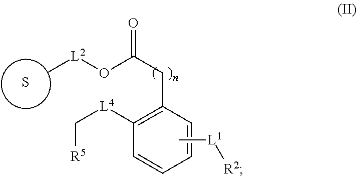

compound (III) has the formula:

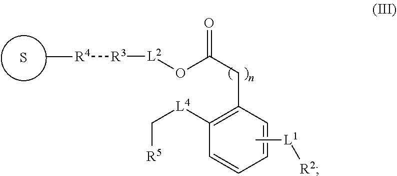

compound (A) has the formula:

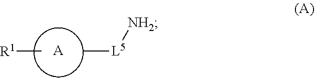

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
n is 0 or 1;
---- is a noncovalent or covalent bond; and
S is a solid support.

Embodiment S40. The method of embodiment S39, wherein ---- is a noncovalent bond.

Embodiment S41. The method of one of embodiments S39 to S40, wherein S is a chromatographic material.

Embodiment S42. The method of one of embodiments S39 to S41, wherein Ring A is a C₃-C₆ cycloalkylene, 3 to 6 membered heterocycloalkylene, or phenylene.

Embodiment S43. The method of one of embodiments S39 to S41, wherein Ring A is phenylene.

Embodiment S44. The method of one of embodiments S39 to S43, wherein L⁴ is a bond.

Embodiment S45. The method of one of embodiments S39 to S44, wherein L⁵ is a bond.

Embodiment S46. The method of one of embodiments S39 to S45, wherein R⁵ is a halogen,

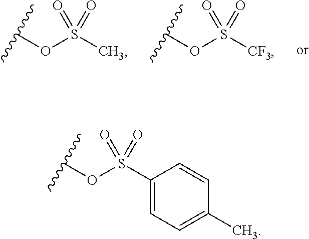 [SMILES: *OS(C)(=O)=O ; *OS(=O)(=O)C(F)(F)F ; "or" ; *OS(=O)(=O)c1ccc(C)cc1]

Embodiment S47. The method of one of embodiments S39 to S45, wherein R⁵ is —Br.

Embodiment S48. The method of one of embodiments S39 to S47, wherein n is 0.

Embodiment S49. The method of one of embodiments S39 to S47, wherein n is 1.

Embodiment S50. The method of one of embodiments S39 to S47, wherein
compound (I) has the formula:

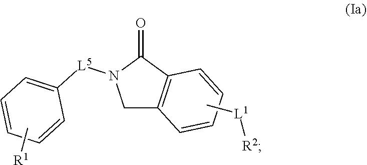

compound (II) has the formula:

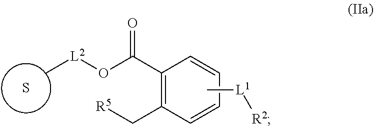

and
compound (III) has the formula:

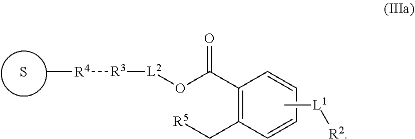

Embodiment S51. The method of one of embodiments S39 to S47, wherein
compound (I) has the formula:

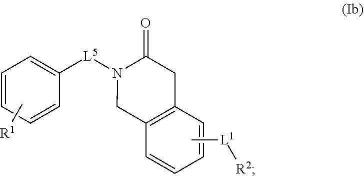

compound (II) has the formula:

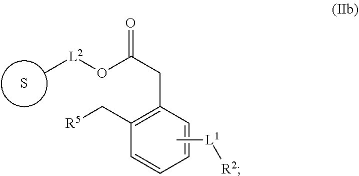

and
compound (III) has the formula:

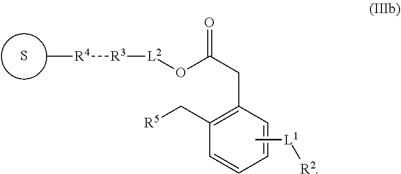

Embodiment S52. The method of one of embodiments S39 to S51, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment S53. The method of one of embodiments S39 to S51, wherein L¹ is —NHC(O)—.

Embodiment S54. The method of one of embodiments S39 to S53, wherein L² is a substituted or unsubstituted alkylene or substituted or unsubstituted heteroalkylene.

Embodiment S55. The method of one of embodiments S39 to S53, wherein L² is an unsubstituted C₁-C₄ alkylene.

Embodiment S56. The method of one of embodiments S39 to S53, wherein L² is an unsubstituted methylene.

Embodiment S57. The method of one of embodiments S39 to S56, wherein R¹ is —¹⁸F.

Embodiment S58. The method of one of embodiments S39 to S57, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment S59. The method of one of embodiments S39 to S57, wherein R² is a monovalent form of a drug or substituted heteroalkyl.

Embodiment S60. The method of one of embodiments S39 to S57, wherein R² is a monovalent form of a drug.

### VII. Additional Embodiments

Embodiment 1. A method of making compound (I^{B}), or a pharmaceutically acceptable salt thereof, said method comprising mixing compound (IV^{B}) or compound (V^{B}) and compound (A) together in a reaction vessel; wherein
compound (I^{B}) has the formula:

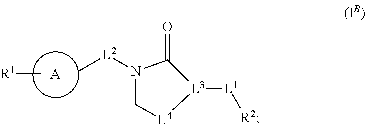

compound (IV^{B}) has the formula:

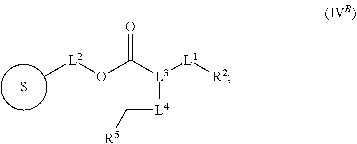

compound (V^{B}) has the formula:

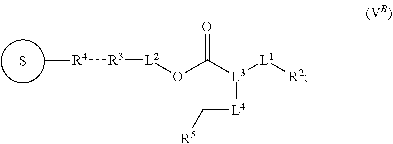

compound (A) has the formula:

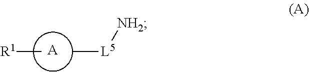

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
---- is a noncovalent or covalent bond; and
S is a solid support.

Embodiment 2. The method of embodiment 1, wherein
compound (I^{B}) has the formula:

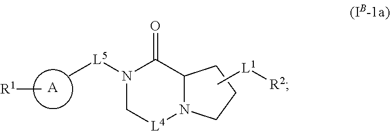

compound (IV^{B}) has the formula:

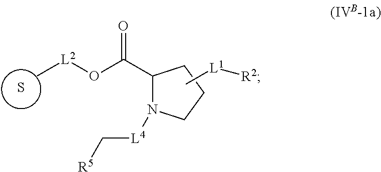

and
compound (V^{B}) has the formula:

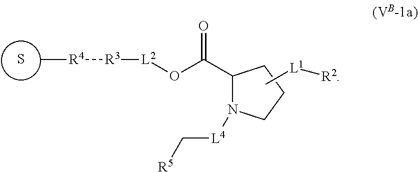

Embodiment 3. The method of one of embodiments 1 to 2, wherein R⁵ is a halogen,

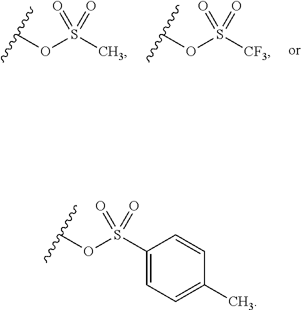 [SMILES: *OS(C)(=O)=O ; *OS(=O)(=O)C(F)(F)F ; "or" ; *OS(=O)(=O)c1ccc(C)cc1]

Embodiment 4. The method of one of embodiments 1 to 2, wherein R⁵ is —Br.

Embodiment 5. The method of one of embodiments 1 to 4, wherein ---- is a noncovalent bond.

Embodiment 6. The method of one of embodiments 1 to 5, wherein S is a chromatographic material.

Embodiment 7. The method of one of embodiments 1 to 6, wherein Ring A is a C₃-C₆ cycloalkylene, 3 to 6 membered heterocycloalkylene, or phenylene.

Embodiment 8. The method of one of embodiments 1 to 6, wherein Ring A is phenylene.

Embodiment 9. The method of one of embodiments 1 to 8, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 10. The method of one of embodiments 1 to 8, wherein L¹ is —NHC(O)—.

Embodiment 11. The method of one of embodiments 1 to 10, wherein L² is a substituted or unsubstituted alkylene or substituted or unsubstituted heteroalkylene.

Embodiment 12. The method of one of embodiments 1 to 10, wherein L² is an unsubstituted C₁-C₄ alkylene.

Embodiment 13. The method of one of embodiments 1 to 10, wherein L² is an unsubstituted methylene.

Embodiment 14. The method of one of embodiments 1 to 13, wherein L⁴ is —C(O)—.

Embodiment 15. The method of one of embodiments 1 to 14, wherein L⁵ is a bond, —NH—, —O—, or —C(O)NH—.

Embodiment 16. The method of one of embodiments 1 to 15, wherein R¹ is —¹⁸F.

Embodiment 17. The method of one of embodiments 1 to 16, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 18. The method of one of embodiments 1 to 16, wherein R² is a monovalent form of a drug or substituted heteroalkyl.

Embodiment 19. The method of one of embodiments 1 to 16, wherein R² is a monovalent form of a drug.

Embodiment 20. A compound, or a pharmaceutically acceptable salt thereof, having the formula:

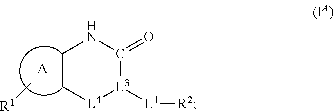

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4; and
m2 and v2 are each independently 1 or 2.

Embodiment 21. The compound of embodiment 20, wherein L³ is an unsubstituted heterocycloalkylene.

Embodiment 22. The compound of one of embodiments 20 to 21, wherein Ring A is a C₃-C₆ cycloalkylene, 3 to 6 membered heterocycloalkylene, or phenylene.

Embodiment 23. The compound of one of embodiments 20 to 21, wherein Ring A is phenylene.

Embodiment 24. The compound of embodiment 20, having the formula:

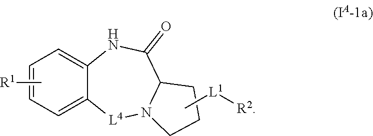

Embodiment 25. The compound of one of embodiments 20 to 24, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 26. The compound of one of embodiments 20 to 24, wherein L¹ is —NHC(O)—.

Embodiment 27. The compound of one of embodiments 20 to 26, wherein L⁴ is —C(O)— or unsubstituted methylene.

Embodiment 28. The compound of one of embodiments 20 to 26, wherein L⁴ is —C(O)—.

Embodiment 29. The compound of one of embodiments 20 to 28, wherein R¹ is —¹⁸F.

Embodiment 30. The compound of one of embodiments 20 to 29, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 31. The compound of one of embodiments 20 to 29, wherein R² is a monovalent form of a drug or substituted heteroalkyl.

Embodiment 32. The compound of one of embodiments 20 to 29, wherein R² is a monovalent form of a drug.

Embodiment 33. A pharmaceutical composition comprising a compound of one of embodiments 20 to 32, or a pharmaceutically acceptable salt thereof, and a pharmaceutically acceptable excipient.

Embodiment 34. A method of detecting a level of a compound in a subject, the method comprising:
(i) administering to the subject a compound of one of embodiments 20 to 32, or a pharmaceutically acceptable salt thereof, and
(ii) detecting the level of the compound in the subject.

Embodiment 35. The method of embodiment 34, wherein step (ii) further comprises detecting the level of the compound in the subject using positron emission tomography (PET), wherein R¹ is a PET detectable radioisotope.

Embodiment 36. The method of embodiment 35, further comprising detecting a physiological location of the compound in the subject using PET.

Embodiment 37. A method of detecting the level of CD44v6 in a subject, the method comprising administering to the subject an effective amount of a compound of one of embodiments 20 to 32, or a pharmaceutically acceptable salt thereof.

Embodiment 38. The method of embodiment 37, further comprising detecting the level of the compound using positron emission tomography.

Embodiment 39. A compound, or a pharmaceutically acceptable salt thereof, having the formula:

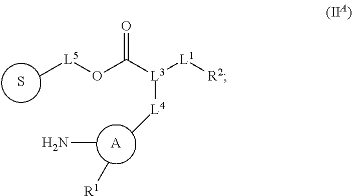

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
S is a solid support.

Embodiment 40. The compound of embodiment 39, wherein L³ is an unsubstituted heterocycloalkylene.

Embodiment 41. The compound of one of embodiments 39 to 40, wherein Ring A is a C₃-C₆ cycloalkylene, 3 to 6 membered heterocycloalkylene, or phenylene.

Embodiment 42. The compound of one of embodiments 39 to 40, wherein Ring A is phenylene.

Embodiment 43. The compound of embodiment 39, having the formula:

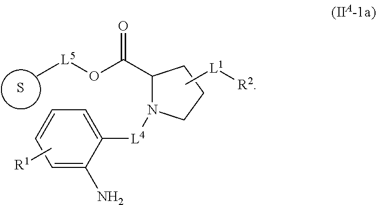

Embodiment 44. A compound, or a pharmaceutically acceptable salt thereof, having the formula:

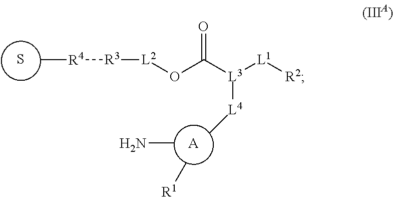

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment 45. The compound of embodiment 44, wherein L³ is an unsubstituted heterocycloalkylene.

Embodiment 46. The compound of one of embodiments 44 to 45, wherein Ring A is a C₃-C₆ cycloalkylene, 3 to 6 membered heterocycloalkylene, or phenylene.

Embodiment 47. The compound of one of embodiments 44 to 45, wherein Ring A is phenylene.

Embodiment 48. The compound of embodiment 44, having the formula:

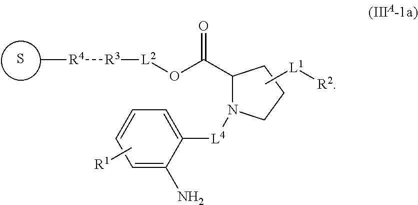

Embodiment 49. The compound of one of embodiments 44 to 48, wherein ---- is a noncovalent bond.

Embodiment 50. The compound of one of embodiments 39 to 49, wherein S is a chromatographic material.

Embodiment 51. The compound of one of embodiments 39 to 50, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 52. The compound of one of embodiments 39 to 50, wherein L¹ is —NHC(O)—.

Embodiment 53. The compound of one of embodiments 39 to 52, wherein L² is a substituted or unsubstituted alkylene or substituted or unsubstituted heteroalkylene.

Embodiment 54. The compound of one of embodiments 39 to 52, wherein L² is an unsubstituted C₁-C₄ alkylene.

Embodiment 55. The compound of one of embodiments 39 to 52, wherein L² is an unsubstituted methylene.

Embodiment 56. The compound of one of embodiments 39 to 55, wherein L⁴ is —C(O)— or unsubstituted methylene.

Embodiment 57. The compound of one of embodiments 39 to 55, wherein L⁴ is —C(O)—.

Embodiment 58. The compound of one of embodiments 39 to 55, wherein R¹ is —¹⁸F.

Embodiment 59. The compound of one of embodiments 39 to 58, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 60. The compound of one of embodiments 39 to 58, wherein R² is a monovalent form of a drug or substituted heteroalkyl.

Embodiment 61. The compound of one of embodiments 39 to 58, wherein R² is a monovalent form of a drug.

Embodiment 62. A method of making compound (I^{A}), or a pharmaceutically acceptable salt thereof, said method comprising mixing compound (II^{A}) or compound (III^{A}) in a reaction vessel; wherein
compound (I^{A}) has the formula:

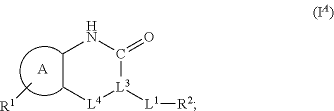

compound (II^{A}) has the formula:

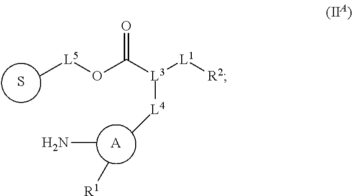

compound (III^{A}) has the formula:

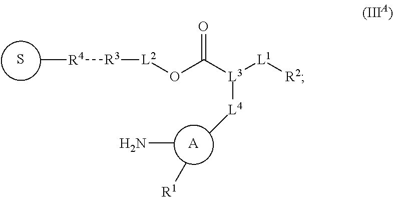

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment 63. The method of embodiment 62, further comprising mixing compound (II^{A}) or compound (III^{A}) in a basic solution.

Embodiment 64. The method of embodiment 63, wherein the basic solution has a pH from 7.1 to 8.

Embodiment 65. The method of one of embodiments 63 to 64, wherein the basic solution has a pH of 7.4.

Embodiment 66. The method of one of embodiments 62 to 65, wherein L³ is an unsubstituted heterocycloalkylene.

Embodiment 67. The method of one of embodiments 62 to 66, wherein Ring A is a C₃-C₆ cycloalkylene, 3 to 6 membered heterocycloalkylene, or phenylene.

Embodiment 68. The method of one of embodiments 62 to 66, wherein Ring A is phenylene.

Embodiment 69. The method of one of embodiments 62 to 65, wherein
compound (I^{A}) has the formula:

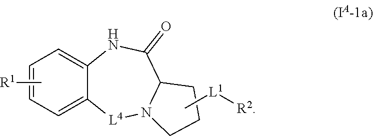

compound (II^{A}) has the formula:

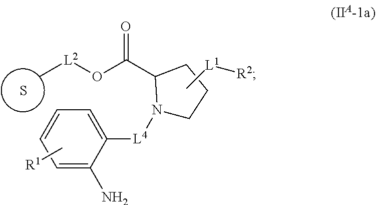

and
compound (III^{A}) has the formula:

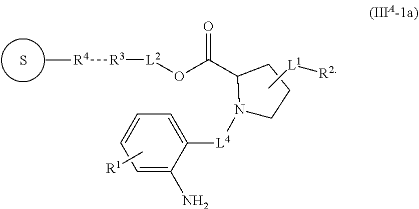

Embodiment 70. The method of one of embodiments 62 to 69, wherein ---- is a noncovalent bond.

Embodiment 71. The method of one of embodiments 62 to 70, wherein S is a chromatographic material.

Embodiment 72. The method of one of embodiments 62 to 71, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 73. The method of one of embodiments 62 to 71, wherein L¹ is —NHC(O)—.

Embodiment 74. The method of one of embodiments 62 to 73, wherein L² is a substituted or unsubstituted alkylene or substituted or unsubstituted heteroalkylene.

Embodiment 75. The method of one of embodiments 62 to 73, wherein L² is an unsubstituted C₁-C₄ alkylene.

Embodiment 76. The method of one of embodiments 62 to 73, wherein L² is an unsubstituted methylene.

Embodiment 77. The method of one of embodiments 62 to 76, wherein L⁴ is —C(O)— or unsubstituted methylene.

Embodiment 78. The method of one of embodiments 62 to 76, wherein L⁴ is —C(O)—.

Embodiment 79. The method of one of embodiments 62 to 78, wherein R¹ is —¹⁸F.

Embodiment 80. The method of one of embodiments 62 to 79, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 81. The method of one of embodiments 62 to 79, wherein R² is a monovalent form of a drug or substituted heteroalkyl.

Embodiment 82. The method of one of embodiments 62 to 79, wherein R² is a monovalent form of a drug.

Embodiment 83. A compound, or a pharmaceutically acceptable salt thereof, having the formula:

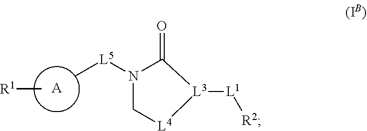

wherein
Ring A is a C₃-C₆ cycloalkylene, 3 to 6 membered heterocycloalkylene, phenylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4; and
m2 and v2 are each independently 1 or 2.

Embodiment 84. The compound of embodiment 83, wherein L³ is an unsubstituted heterocycloalkylene.

Embodiment 85. The compound of embodiment 83, having the formula:

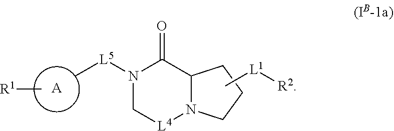

Embodiment 86. The compound of one of embodiments 83 to 85, wherein Ring A is a C₃-C₆ cycloalkylene, 3 to 6 membered heterocycloalkylene, or phenylene.

Embodiment 87. The compound of one of embodiments 83 to 85, wherein Ring A is phenylene.

Embodiment 88. The compound of one of embodiments 83 to 87, wherein L⁴ is —C(O)—.

Embodiment 89. The compound of one of embodiments 83 to 88, wherein L⁵ is a bond, —NH—, —O—, or —C(O)NH—.

Embodiment 90. The compound of one of embodiments 83 to 89, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 91. The compound of one of embodiments 83 to 89, wherein L¹ is —NHC(O)—.

Embodiment 92. The compound of one of embodiments 83 to 91, wherein R¹ is —¹⁸F.

Embodiment 93. The compound of one of embodiments 83 to 92, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 94. The compound of one of embodiments 83 to 92, wherein R² is a monovalent form of a drug or substituted heteroalkyl.

Embodiment 95. The compound of one of embodiments 83 to 92, wherein R² is a monovalent form of a drug.

Embodiment 96. A pharmaceutical composition comprising a compound of one of embodiments 83 to 95, or a pharmaceutically acceptable salt thereof, and a pharmaceutically acceptable excipient.

Embodiment 97. A method of detecting a level of a compound in a subject, the method comprising: (i) administering to the subject a compound of one of embodiments 83 to 95, or a pharmaceutically acceptable salt thereof, and (ii) detecting the level of the compound in the subject.

Embodiment 98. The method of embodiment 97, wherein step (ii) further comprises detecting the level of the compound in the subject using positron emission tomography (PET), wherein R¹ is a PET detectable radioisotope.

Embodiment 99. The method of embodiment 98, further comprising detecting a physiological location of the compound in the subject using PET.

Embodiment 100. A method of detecting the level of CD44v6 in a subject, the method comprising administering to the subject an effective amount of a compound of one of embodiments 83 to 95, or a pharmaceutically acceptable salt thereof.

Embodiment 101. The method of embodiment 100, further comprising detecting the level of the compound using positron emission tomography.

Embodiment 102. A compound, or a pharmaceutically acceptable salt thereof, having the formula:

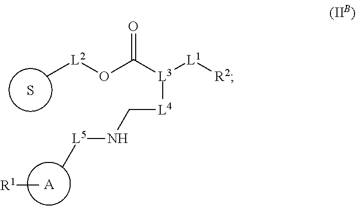

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
S is a solid support.

Embodiment 103. The compound of embodiment 102, having the formula:

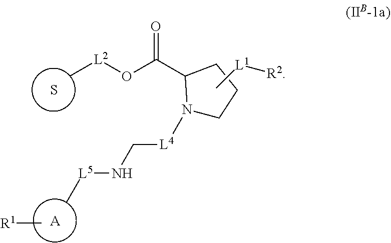

Embodiment 104. A compound, or a pharmaceutically acceptable salt thereof, having the formula:

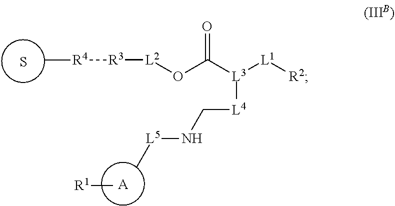

wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR²C, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment 105. The compound of embodiment 104, having the formula:

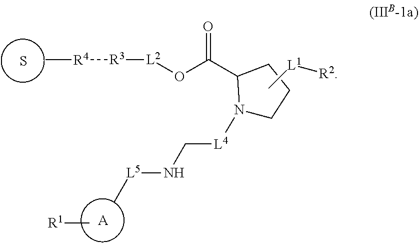

Embodiment 106. The compound of one of embodiments 104 to 105, wherein ---- is a noncovalent bond.

Embodiment 107. The compound of one of embodiments 102 to 106, wherein Ring A is a C₃-C₆ cycloalkylene, 3 to 6 membered heterocycloalkylene, or phenylene.

Embodiment 108. The compound of one of embodiments 102 to 106, wherein Ring A is phenylene.

Embodiment 109. The compound of one of embodiments 102 to 108, wherein R¹ is —¹⁸F.

Embodiment 110. A compound, or a pharmaceutically acceptable salt thereof, having the formula:

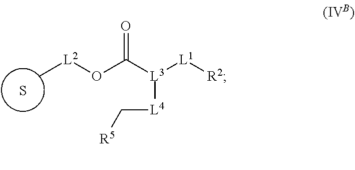

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R⁵ is a leaving group; and
S is a solid support.

Embodiment 111. The compound of embodiment 110, having the formula:

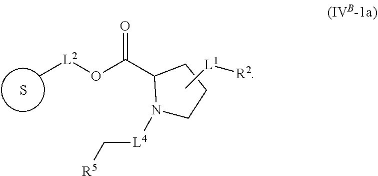

Embodiment 112. A compound, or a pharmaceutically acceptable salt thereof, having the formula:

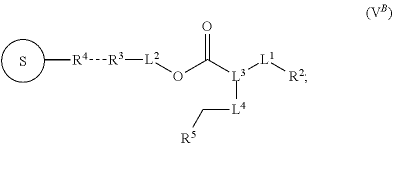

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
---- is a noncovalent or covalent bond; and
S is a solid support.

Embodiment 113. The compound of embodiment 112, having the formula:

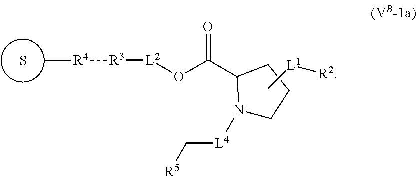

Embodiment 114. The compound of one of embodiments 112 to 113, wherein ---- is a noncovalent bond.

Embodiment 115. The compound of one of embodiments 110 to 114, wherein R⁵ is a halogen,

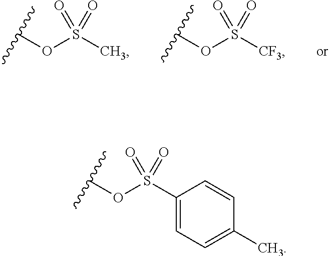 [SMILES: *OS(C)(=O)=O ; *OS(=O)(=O)C(F)(F)F ; *OS(=O)(=O)c1ccc(C)cc1]

Embodiment 116. The compound of one of embodiments 110 to 114, wherein R⁵ is —Br.

Embodiment 117. The compound of one of embodiments 102 to 116, wherein S is a chromatographic material.

Embodiment 118. The compound of one of embodiments 102 to 117, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 119. The compound of one of embodiments 102 to 117, wherein L¹ is —NHC(O)—.

Embodiment 120. The compound of one of embodiments 102 to 119, wherein L² is a substituted or unsubstituted alkylene or substituted or unsubstituted heteroalkylene.

Embodiment 121. The compound of one of embodiments 102 to 119, wherein L² is an unsubstituted C₁-C₄ alkylene.

Embodiment 122. The compound of one of embodiments 102 to 119, wherein L² is an unsubstituted methylene.

Embodiment 123. The compound of one of embodiments 102 to 122, wherein L⁴ is —C(O)—.

Embodiment 124. The compound of one of embodiments 102 to 123, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 125. The compound of one of embodiments 102 to 123, wherein R² is a monovalent form of a drug or substituted heteroalkyl.

Embodiment 126. The compound of one of embodiments 102 to 123, wherein R² is a monovalent form of a drug.

Embodiment 127. A method of making compound (I^{B}), or a pharmaceutically acceptable salt thereof, said method comprising mixing compound (II^{B}) or compound (III^{B}) in a reaction vessel; wherein
compound (I^{B}) has the formula:

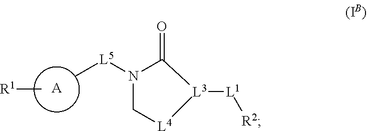

compound (II^{B}) has the formula:

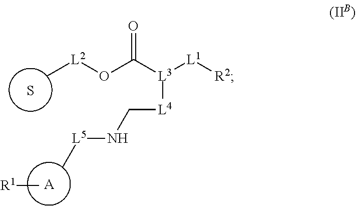

compound (III^{B}) has the formula:

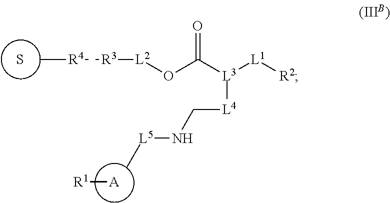

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene; L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

Embodiment 128. The method of embodiment 127, wherein
compound (I^{B}) has the formula:

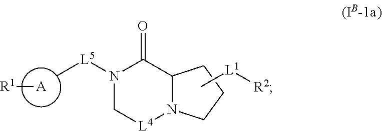

compound (II^{B}) has the formula:

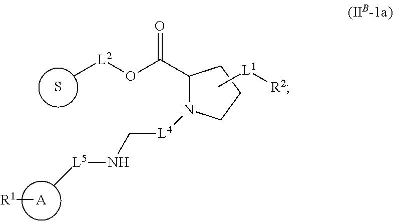

and
compound (III^{B}) has the formula:

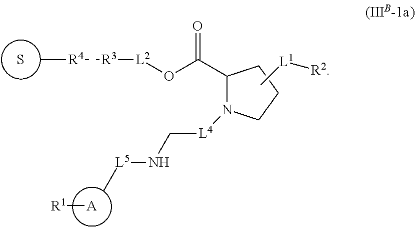

Embodiment 129. The method of one of embodiments 127 to 128, further comprising mixing compound (II^{B}) or compound (III^{B}) in a basic solution.

Embodiment 130. The method of embodiment 129, wherein the basic solution has a pH from 7.1 to 8.

Embodiment 131. The method of one of embodiments 129 to 130, wherein the basic solution has a pH of 7.4.

Embodiment 132. The method of one of embodiments 127 to 131, wherein ---- is a noncovalent bond.

Embodiment 133. The method of one of embodiments 127 to 132, wherein S is a chromatographic material.

Embodiment 134. The method of one of embodiments 127 to 133, wherein Ring A is a C₃-C₆ cycloalkylene, 3 to 6 membered heterocycloalkylene, or phenylene.

Embodiment 135. The method of one of embodiments 127 to 133, wherein Ring A is phenylene.

Embodiment 136. The method of one of embodiments 127 to 135, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 137. The method of one of embodiments 127 to 135, wherein L¹ is —NHC(O)—.

Embodiment 138. The method of one of embodiments 127 to 137, wherein L² is a substituted or unsubstituted alkylene or substituted or unsubstituted heteroalkylene.

Embodiment 139. The method of one of embodiments 127 to 137, wherein L² is an unsubstituted C₁-C₄ alkylene.

Embodiment 140. The method of one of embodiments 127 to 137, wherein L² is an unsubstituted methylene.

Embodiment 141. The method of one of embodiments 127 to 140, wherein L⁴ is —C(O)—.

Embodiment 142. The method of one of embodiments 127 to 141, wherein L⁵ is a bond, —NH—, —O—, or —C(O)NH—.

Embodiment 143. The method of one of embodiments 127 to 142, wherein R¹ is —¹⁸F.

Embodiment 144. The method of one of embodiments 127 to 143, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 145. The method of one of embodiments 127 to 143, wherein R² is a monovalent form of a drug or substituted heteroalkyl.

Embodiment 146. The method of one of embodiments 127 to 143, wherein R² is a monovalent form of a drug.

Embodiment 147. A compound, or a pharmaceutically acceptable salt thereof, having the formula:

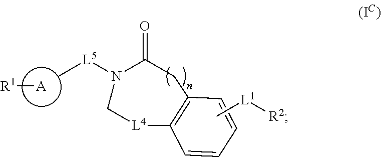

wherein
Ring A is a C₃-C₆ cycloalkylene, 3 to 6 membered heterocycloalkylene, phenylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
n is 0 or 1.

Embodiment 148. The compound of embodiment 147, wherein Ring A is a C₃-C₆ cycloalkylene, 3 to 6 membered heterocycloalkylene, or phenylene.

Embodiment 149. The compound of embodiment 147, wherein Ring A is phenylene.

Embodiment 150. The compound of one of embodiments 147 to 149, wherein L⁴ is a bond.

Embodiment 151. The compound of one of embodiments 147 to 150, wherein L⁵ is a bond.

Embodiment 152. The compound of one of embodiments 147 to 151, wherein n is 0.

Embodiment 153. The compound of one of embodiments 147 to 151, wherein n is 1.

Embodiment 154. The compound of embodiment 147, having the formula:

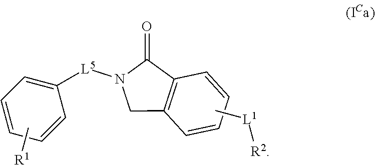

Embodiment 155. The compound of embodiment 147, having the formula:

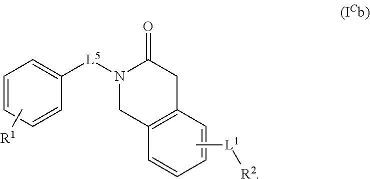

Embodiment 156. The compound of one of embodiments 147 to 155, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 157. The compound of one of embodiments 147 to 155, wherein L¹ is —NHC(O)—.

Embodiment 158. The compound of one of embodiments 147 to 157, wherein R¹ is —¹⁸F.

Embodiment 159. The compound of one of embodiments 147 to 158, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 160. The compound of one of embodiments 147 to 159, wherein R² is a monovalent form of a drug or substituted heteroalkyl.

Embodiment 161. The compound of one of embodiments 147 to 159, wherein R² is a monovalent form of a drug.

Embodiment 162. A pharmaceutical composition comprising a compound of one of embodiments 147 to 161, or a pharmaceutically acceptable salt thereof, and a pharmaceutically acceptable excipient.

Embodiment 163. A method of detecting the level of a compound in a subject, the method comprising:
(i) administering to the subject a compound of one of embodiments 147 to 161, or a pharmaceutically acceptable salt thereof, and
(ii) detecting the level of the compound in the subject.

Embodiment 164. The method of embodiment 163, wherein step (ii) further comprises detecting the level of the compound in the subject using positron emission tomography (PET), wherein R¹ is a PET detectable radioisotope.

Embodiment 165. The method of embodiment 164, further comprising detecting a physiological location of the compound in the subject using PET.

Embodiment 166. A method of detecting the level of CD44v6 in a subject, the method comprising administering to the subject an effective amount of a compound of one of embodiments 147 to 161, or a pharmaceutically acceptable salt thereof.

Embodiment 167. The method of embodiment 166, further comprising detecting the level of the compound using positron emission tomography.

Embodiment 168. A compound, or a pharmaceutically acceptable salt thereof, having the formula:

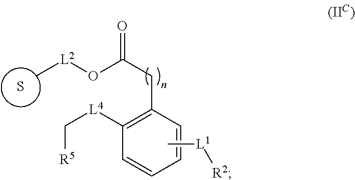

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R⁵ is a leaving group;
n is 0 or 1; and
S is a solid support.

Embodiment 169. A compound, or a pharmaceutically acceptable salt thereof, having the formula:

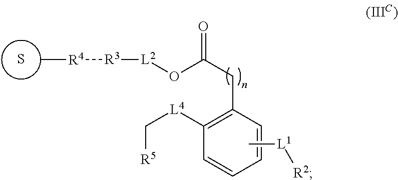

wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
n is 0 or 1;
---- is a noncovalent or covalent bond; and
S is a solid support.

Embodiment 170. The compound of embodiment 169, wherein ---- is a noncovalent bond.

Embodiment 171. The compound of one of embodiments 168 to 170, wherein S is a chromatographic material.

Embodiment 172. The compound of one of embodiments 168 to 171, wherein n is 0.

Embodiment 173. The compound of one of embodiments 168 to 171, wherein n is 1.

Embodiment 174. The compound of one of embodiments 168 to 173, wherein R⁵ is a halogen,

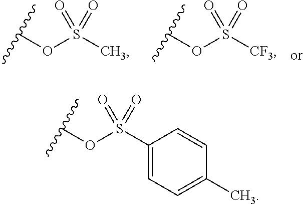 [SMILES: *OS(C)(=O)=O ; *OS(=O)(=O)C(F)(F)F ; *OS(=O)(=O)c1ccc(C)cc1]

Embodiment 175. The compound of one of embodiments 168 to 173, wherein R⁵ is —Br.

Embodiment 176. The compound of one of embodiments 168 to 175, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 177. The compound of one of embodiments 168 to 175, wherein L¹ is —NHC(O)—.

Embodiment 178. The compound of one of embodiments 168 to 177, wherein L² is a substituted or unsubstituted alkylene or substituted or unsubstituted heteroalkylene.

Embodiment 179. The compound of one of embodiments 168 to 177, wherein L² is an unsubstituted C₁-C₄ alkylene.

Embodiment 180. The compound of one of embodiments 168 to 177, wherein L² is an unsubstituted methylene.

Embodiment 181. The compound of one of embodiments 168 to 180, wherein L⁴ is a bond.

Embodiment 182. The compound of one of embodiments 168 to 181, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 183. The compound of one of embodiments 168 to 181, wherein R² is a monovalent form of a drug or substituted heteroalkyl.

Embodiment 184. The compound of one of embodiments 168 to 181, wherein R² is a monovalent form of a drug.

Embodiment 185. A method of making compound (I^{C}), or a pharmaceutically acceptable salt thereof, said method comprising mixing compound (II^{C}) or compound (III^{C}) and compound (A) together in a reaction vessel; wherein
compound (I^{C}) has the formula:

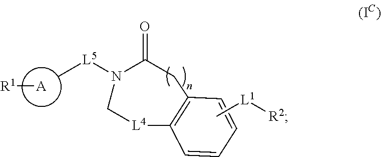

compound (II^{C}) has the formula:

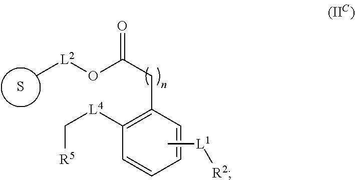

compound (III^{C}) has the formula:

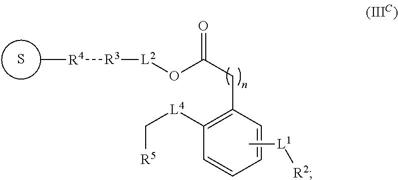

compound (A) has the formula:

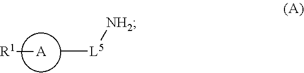

Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
n is 0 or 1;
---- is a noncovalent or covalent bond; and
S is a solid support.

Embodiment 186. The method of embodiment 185, wherein ---- is a noncovalent bond.

Embodiment 187. The method of one of embodiments 185 to 186, wherein S is a chromatographic material.

Embodiment 188. The method of one of embodiments 185 to 187, wherein Ring A is a C₃-C₆ cycloalkylene, 3 to 6 membered heterocycloalkylene, or phenylene.

Embodiment 189. The method of one of embodiments 185 to 187, wherein Ring A is phenylene.

Embodiment 190. The method of one of embodiments 185 to 189, wherein L⁴ is a bond.

Embodiment 191. The method of one of embodiments 185 to 190, wherein L⁵ is a bond.

Embodiment 192. The method of one of embodiments 185 to 191, wherein R⁵ is a halogen,

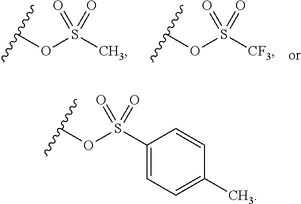 [SMILES: *OS(C)(=O)=O ; *OS(=O)(=O)C(F)(F)F ; *OS(=O)(=O)c1ccc(C)cc1]

Embodiment 193. The method of one of embodiments 185 to 191, wherein R⁵ is —Br.

Embodiment 194. The method of one of embodiments 185 to 193, wherein n is 0.

Embodiment 195. The method of one of embodiments 185 to 193, wherein n is 1.

Embodiment 196. The method of one of embodiments 185 to 193, wherein
compound (I^{C}) has the formula:

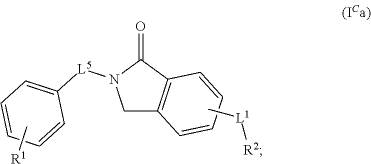

compound (II^{C}) has the formula:

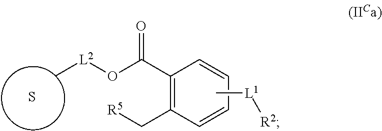

and
compound (III^{C}) has the formula:

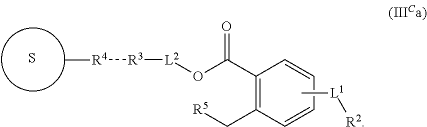

Embodiment 197. The method of one of embodiments 185 to 193, wherein
compound (I^{C}) has the formula:

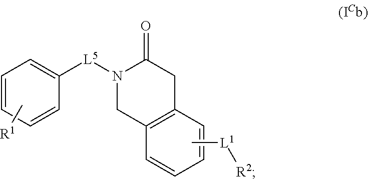

compound (II^{C}) has the formula:

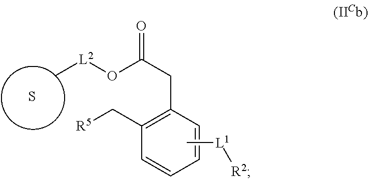

and
compound (III^{C}) has the formula:

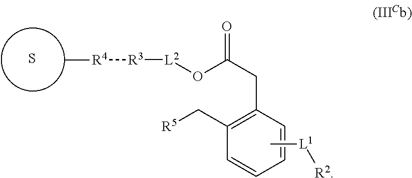

Embodiment 198. The method of one of embodiments 185 to 197, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

Embodiment 199. The method of one of embodiments 185 to 197, wherein L¹ is —NHC(O)—.

Embodiment 200. The method of one of embodiments 185 to 199, wherein L² is a substituted or unsubstituted alkylene or substituted or unsubstituted heteroalkylene.

Embodiment 201. The method of one of embodiments 185 to 199, wherein L² is an unsubstituted C₁-C₄ alkylene.

Embodiment 202. The method of one of embodiments 185 to 199, wherein L² is an unsubstituted methylene.

Embodiment 203. The method of one of embodiments 185 to 202, wherein R¹ is —¹⁸F.

Embodiment 204. The method of one of embodiments 185 to 203, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 205. The method of one of embodiments 185 to 203, wherein R² is a monovalent form of a drug or substituted heteroalkyl.

Embodiment 206. The method of one of embodiments 185 to 203, wherein R² is a monovalent form of a drug.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Imaging Aggressive Prostate Cancer-Associated CD44v6 Using Radiolabeled Resin Cleavage Peptides

Described herein, inter alia, is a process by which a peptide or peptide-like molecule is obtained in high purity, high molar activity in an injection-ready formulation without need for purification or reformulation. For example, the peptide is attached to a solid support or an affinity chromatography group (i.e., biotin) via a diamino acid linker. The solid support or affinity group is attached via ester linkage to the carboxylic acid of the diamino acid linker and the peptide is attached via an amide bond to one of the amines. The linker is reacted with an 18-fluoride labelled amino acid prosthetic to form a covalent amide bond. The prosthetic can then assist in the cleavage of the ester linkage to the solid support or affinity group by spontaneous diketopiperazine formation in pH 7.4 buffered saline, liberating only labelled molecules in high purity and in high molar activity.

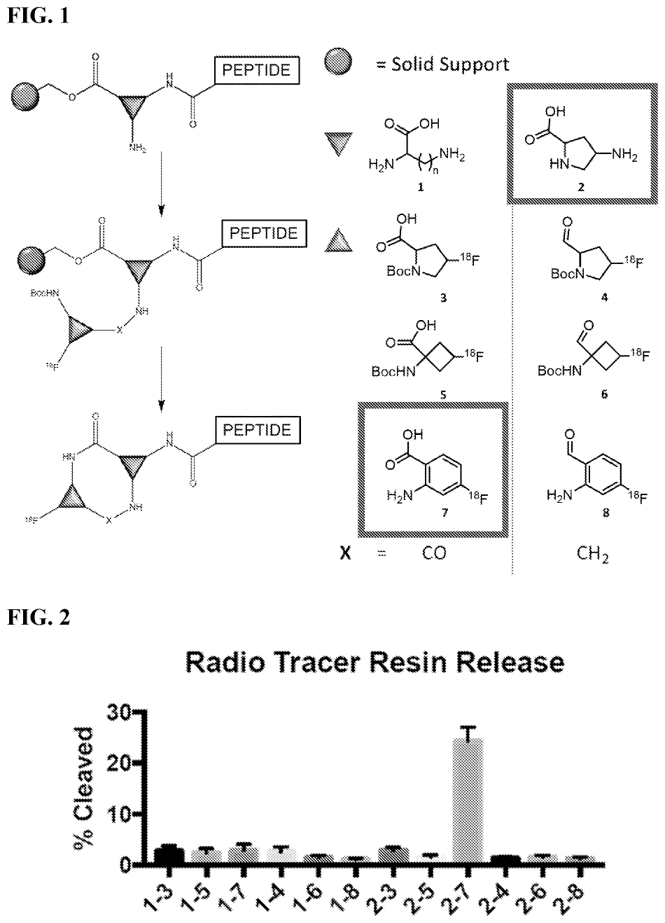

Radiolabel resin cleavage (RRC) of peptides. The strategy entails the synthesis of a radiolabeled prosthetic selected from a panel of structures shown in FIG. 1. Each of the structures is evaluated using “cold” mimicry of the radiolabeling protocol in order to identify the ideal candidate. The radiolabeled prosthetic is synthesized using [¹⁸F]-fluoride provided by the UCSF cyclotron facility. The prosthetics are then used to make radiolabeled analogs of the CD44v6 binding peptides. The peptides themselves are assembled on solid support using conventional solid phase peptides synthesis.

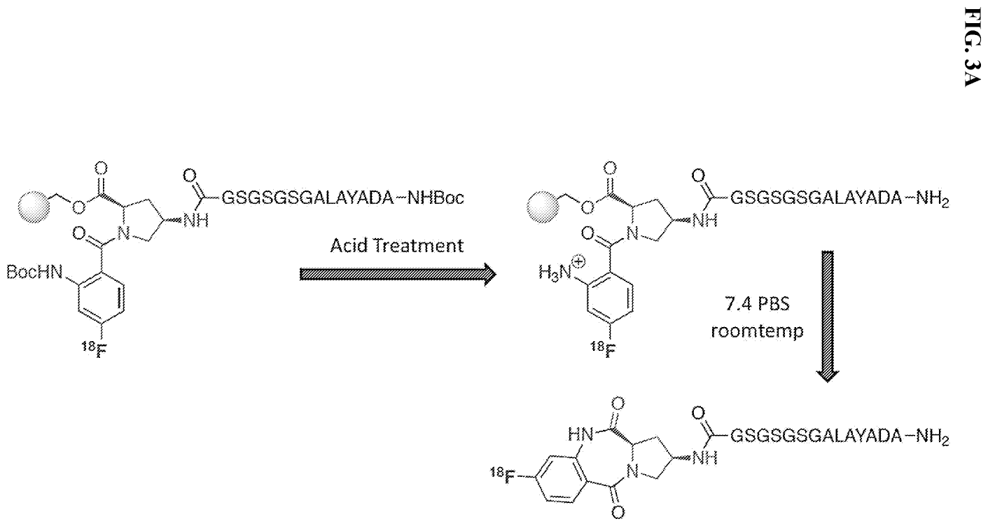

The proposed library of linker/prosthetic combinations were synthesized and their release from solid support was evaluated in pH 7.4 buffered saline at room temperature. Interestingly, one linker/prosthetic combination was far superior to all others, yielding greater than 20% isolated (FIG. 2). That linker/prosthetic combination (FIG. 1—boxed) was utilized to build, radiolabel, and isolate a sample peptide. The radiolabeled peptide was obtained in 17% decay corrected yield and in greater than 95% purity.

All proposed linkers and prosthetics were synthesized from commercially available materials and assembled on solid support to form the library of 12. The library was subjected to pH 7.4 buffer saline at room temperature and the liberated portion was analyzed. From the library, the linker (compound 2)/prosthetic (compound 7) combination demonstrated a superior liberated fraction. The proline linker (compound 2) superior performance is likely due to the tertiary amide bond being less restricted for cis/trans isomerization. 20 kcal/mole is normally required to rotate the secondary amide bond of alpha amino acids into a cis conformation, which is required for the cyclization reaction. The anthranilic acid prosthetic (compound 7) superior performance is likely due to the ˜5 pKa of protonated aniline in comparison to the ˜9 pKa of protonated aliphatic amines. At pH 7.4, nearly all of the aniline is free-base and nucleophilic to perform the cyclization reaction. Conversely, at pH 7.4 all of the aliphatic amine is still protonated, hindering further reactivity. It is noteworthy to point out that the 20% isolated product represents the finished injectable ready material.

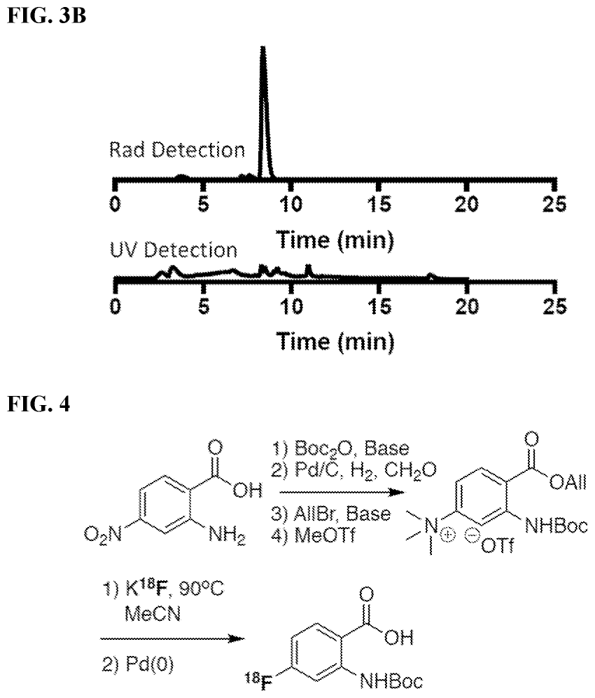

The linker/prosthetic combination was then used for assembling and radiolabeling a sample peptide (FIG. 3A). This peptide was made using amino acids already in possession but does reflect the generality of the approach as the peptide possess aliphatic (Gly and Ala) and aromatic residues (Tyr) as well as alcohol (Ser), amine (Lys), and acid (Asp) side chain bearing residues. The peptide was assembled onto the linker using standard FMOC peptide solid phase synthesis techniques with tert-butyl type side chain protecting groups. The prosthetic was synthesized in 4 steps from commercially available starting materials and subsequently radiolabeled in 2 steps (30 mins) in 37% decay corrected radiochemical yield, in an approach similar to SFB (a commonly used prosthetic) (FIG. 4). The prosthetic was coupled to the peptide and all protecting groups were removed using acid. The resin is then excessively washed and dried, then subjected to pH 7.4 buffer saline at room temperature to liberate 17% of the radiolabeled peptide with greater than 95% purity as evidenced by HPLC within 30 mins (FIG. 3B). The prosthetic synthesis is easy and amenable to automation and the overall synthesis can be applied to readily synthesize multiple tracers from a single prosthetic batch.

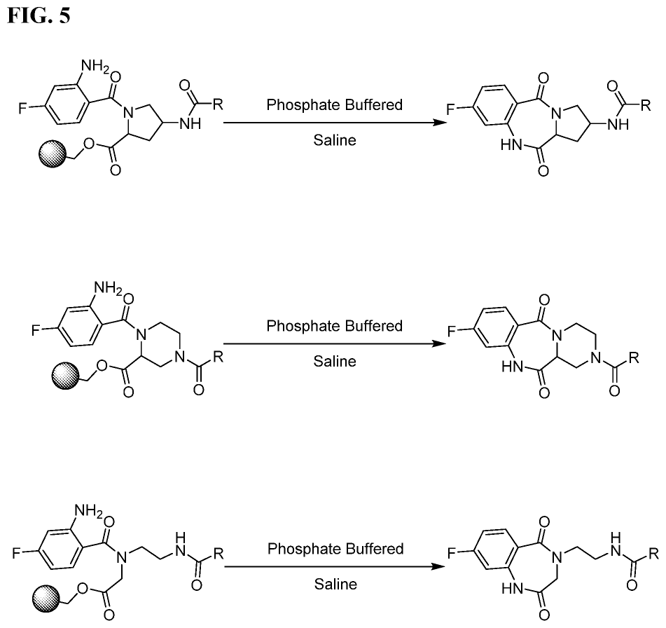

Future work includes using the proposed strategy to make a library of 4 peptides and study them in cells and in animal models for imaging of aggressive prostate cancer. Additionally, the synthetic methodology development includes investigating N-phenyl amino acids (FIG. 5). The combination of compound 2 and compound 7 yields a 7-membered benzodiazepine-dione ring structure. Typically, 5 and 6 membered rings are more kinetically favored. N-phenyl amino acids possess an aniline nitrogen but can form the 6-membered piperazine-dione ring structure, which could make them more amenable to the radiolabeling strategy.

### Example 2: Synthesis and Characterization Data

Chemicals and solvents of reagent grade obtained commercially were of a purity of 95% and used without any further purification. HPLC water was purchased from Fisher. HPLC acetonitrile was purchased from Sigma Aldrich. Synthesis of allyl 2-[(tert-butoxycarbonyl)amino]-4-trimethylaminobenzoate was performed as previously described by Hedou et al. (Tetrahedron Lett., 2015, 56, 27, 4088-4092) and Keenan et al. (J. Med. Chem., 1999, 42, 4, 545-559).

Radiosynthesis of 4-[¹⁸F]aniline was performed as previously described by Tredwell et al. (Angew Chem IE, 2014, 126, 30, 7885-7889).

Radiochemistry synthesis was performed manually with in a lead lined hot cell using aqueous [¹⁸F]fluoride obtained from a cyclotron using ¹⁸O(p,n)¹⁸F nuclear reaction with a 16.5 MeV proton irradiation. Typical experiments were conducted with 100-500 MBq of ¹⁸F radioactivity.

Analytical HPLC was performed on an Waters HPLC with UV detection at 254 nm in series with a γ-detector equipped with a C-18 reversed-phase column (Phenomenex, 4.6×250, 5μ) using gradient of 5-95% acetonitrile in water with 0.1% trifluoroacetic acid over 20 mins followed by 95% acetonitrile in water with 0.1% trifluoroacetic acid over 10 mins. Peaksimple software was used to record and analyze both UV and radiometric data. Radioactivity was quantified using a calibrated Capintec dose calibrator.

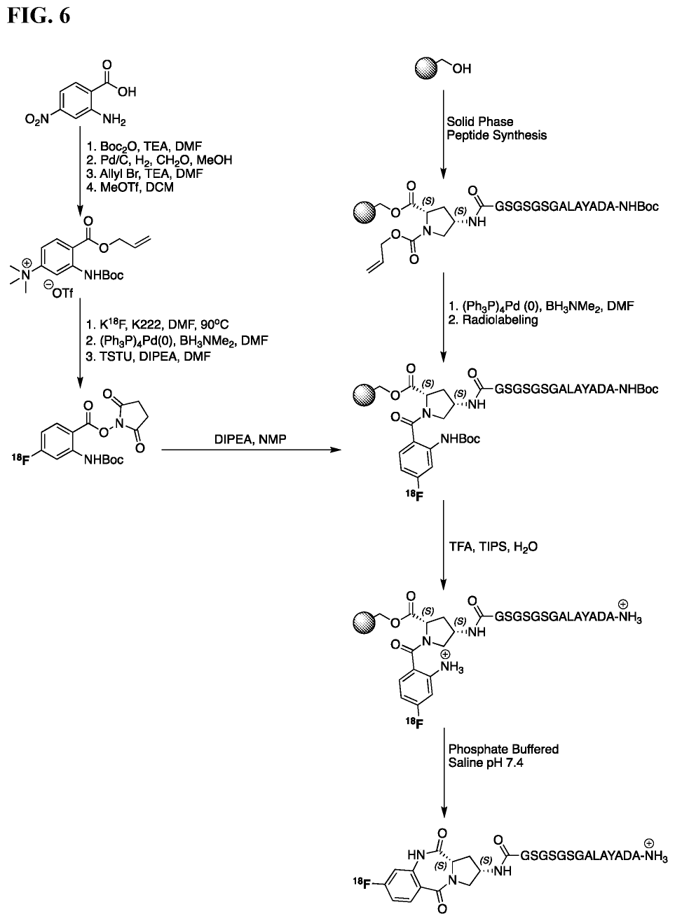

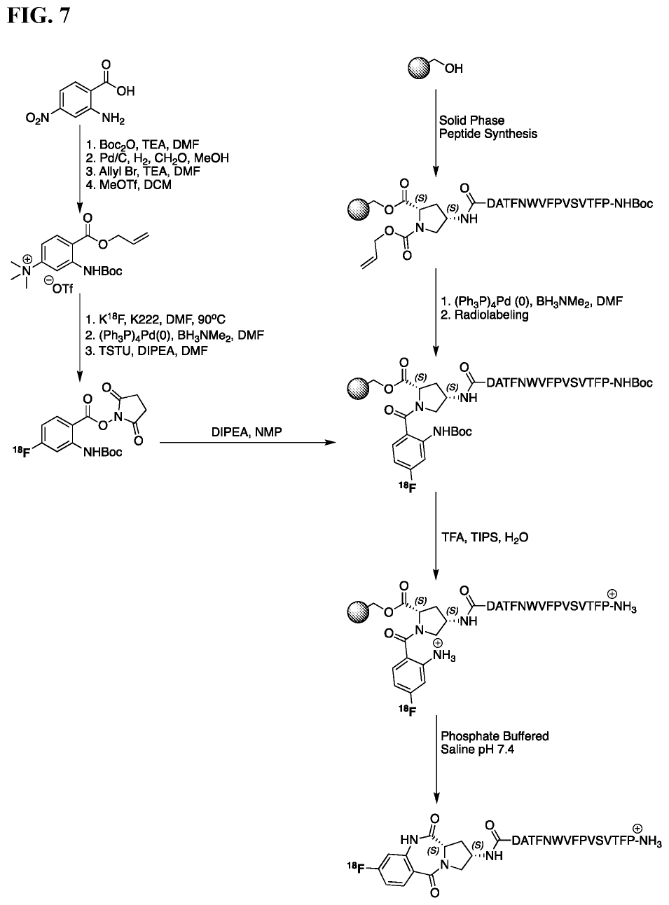

### Example 1: Synthesis of a Solid Supported GSGSGSGALAYADA (SEQ ID NO:1) Peptide (FIG. 6)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling of allyl 2-[(tert-butoxycarbonyl)amino]-4-trimethylaminobenzoate

A MYJA anion exchange column was conditioned with 6 mL of ethanol followed by 6 mL of potassium bicarbonate followed by 6 mL of water. 500 MBq of an aqueous [¹⁸F]fluoride solution was then passed through the cartridge to immobilized the [¹⁸F]fluoride. The [¹⁸F]fluoride was eluted with a mixture of 1 mg potassium bicarbonate 11 mg of Kryptofix 2.2.2 in 800 μL acetonitrile and 100 μL water. The solution was dried under vacuum and at 115° C. 5 mg of allyl 2-[(tert-butoxycarbonyl)amino]-4-trimethylaminobenzoate was added in 800 μL of dimethylformamide. The mixture was heated to 90° C. for 20 mins then cooled. A solution of 1 mg of tetrakis(triphenylphospine)palladium (0) and 11 mg of borane dimethylamine in 500 μL of dimethylformamide. The mixture was agitated for 20 mins then cooled. A solution of 30 mg of N,N,N′,N′-Tetramethyl-O—(N-succinimidyl)uronium tetrafluoroborate and 20 μL of diisopropylethylamine in 1 mL of dimethylformamide was added. The mixture was agitated for 20 mins then cooled.

### Purification of the Synthesized 2-[(Tert-Butoxycarbonyl)Amino]-4-[¹⁸F]fluorobenzoate

The solution was diluted with 15 mL of water and trapped onto an HLB cartridge. The cartridge was washed with 20% acetonitrile in water. The radiolabeled compound was eluted from the cartridge with acetonitrile.

### Radio Ligation of a Solid Supported GSGSGSGALAYADA (SEQ ID NO:1) Peptide

A solution of the 2-[(tert-butoxycarbonyl)amino]-4-[¹⁸F]fluorobenzoate in N-methyl pyrrolidine was added to the solid supported GSGSGSGALAYADA (SEQ ID NO:1) peptide and agitated for 20 minutes. The solution was drained from the resin. The resin was washed with dimethylformamide, dichloromethane, and ethanol. A solution of trifluoroacetic acid, triisopropyl silane, and water was added to the resin and agitated for 20 minutes. The solution was drained from the resin. The resin was washed with ethanol and pH 4 buffer and dried with air. A solution of phosphate buffered saline (pH 7.4) was added to the resin and agitated for 20 minutes. The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 8.4 min). The standard was identified by mass spectrometry (calculated for (M+H+) 1414.5963; measured 1414.5957).

### Example 2: Synthesis of a Solid Supported DATFNWVFPVSVTFP (SEQ ID NO:2) Peptide (FIG. 7)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling of Allyl 2-[(tert-butoxycarbonyl)amino]-4-trimethylaminobenzoate

A MYJA anion exchange column was conditioned with 6 mL of ethanol followed by 6 mL of potassium bicarbonate followed by 6 mL of water. 500 MBq of an aqueous [¹⁸F]fluoride solution was then passed through the cartridge to immobilized the [¹⁸F]fluoride. The [¹⁸F]fluoride was eluted with a mixture of 1 mg potassium bicarbonate 11 mg of Kryptofix 2.2.2 in 800 μL acetonitrile and 100 μL water. The solution was dried under vacuum and at 115° C. 5 mg of allyl 2-[(tert-butoxycarbonyl)amino]-4-trimethylaminobenzoate was added in 800 μL of dimethylformamide. The mixture was heated to 90° C. for 20 mins then cooled. A solution of 1 mg of tetrakis(triphenylphospine)palladium (0) and 11 mg of borane dimethylamine in 500 μL of dimethylformamide. The mixture was agitated for 20 mins then cooled. A solution of 30 mg of N,N,N′,N′-Tetramethyl-O—(N-succinimidyl)uronium tetrafluoroborate and 20 μL of diisopropylethylamine in 1 mL of dimethylformamide was added. The mixture was agitated for 20 mins then cooled.

### Purification of the Synthesized 2-[(tert-butoxycarbonyl)amino]-4-[¹⁸F]fluorobenzoate

The solution was diluted with 15 mL of water and trapped onto an HLB cartridge. The cartridge was washed with 20% acetonitrile in water. The radiolabeled compound was eluted from the cartridge with acetonitrile.

### Radio Ligation of a Solid Supported DATFNWVFPVSVTFP (SEQ ID NO:2) Peptide

A solution of the 2-[(tert-butoxycarbonyl)amino]-4-[¹⁸F]fluorobenzoate in N-methyl pyrrolidine was added to the solid supported DATFNWVFPVSVTFP (SEQ ID NO:2) peptide and agitated for 20 minutes. The solution was drained from the resin. The resin was washed with dimethylformamide, dichloromethane, and ethanol. A solution of trifluoroacetic acid, triisopropyl silane, and water was added to the resin and agitated for 20 minutes. The solution was drained from the resin. The resin was washed with ethanol and pH 4 buffer and dried with air. A solution of phosphate buffered saline (pH 7.4) was added to the resin and agitated for 20 minutes. The solution was collected and analyzed via HPLC to yield the radiolabeled compound in >95% purity with a retention time of 23.1 min.

### Synthesis and Purification of the Cold Standard of the DATFNWVFPVSVTFP (SEQ ID NO:2) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 23.1 min). The standard was identified by mass spectrometry (calculated for (M+H⁺) 1957.9213; measured 1957.9209).

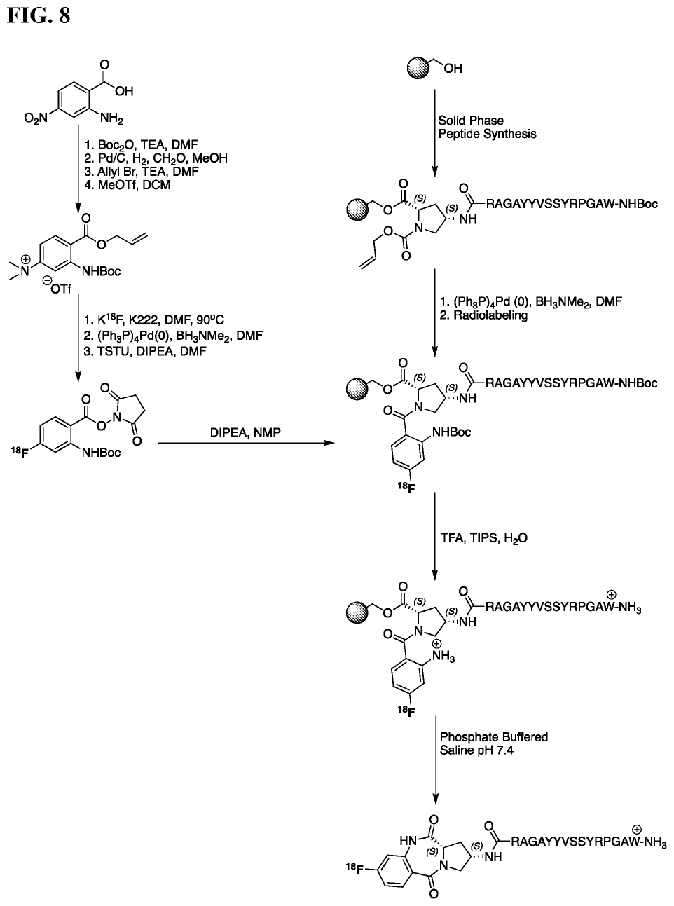

### Example 3: Synthesis of a Solid Supported RAGAYYVSSYRPGAW (SEQ ID NO:3) Peptide (FIG. 8)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling of allyl 2-[(tert-butoxycarbonyl)amino]-4-trimethylaminobenzoate

A MYJA anion exchange column was conditioned with 6 mL of ethanol followed by 6 mL of potassium bicarbonate followed by 6 mL of water. 500 MBq of an aqueous [¹⁸F]fluoride solution was then passed through the cartridge to immobilized the [¹⁸F]fluoride. The [¹⁸F]fluoride was eluted with a mixture of 1 mg potassium bicarbonate 11 mg of Kryptofix 2.2.2 in 800 μL acetonitrile and 100 μL water. The solution was dried under vacuum and at 115° C. 5 mg of allyl 2-[(tert-butoxycarbonyl)amino]-4-trimethylaminobenzoate was added in 800 μL of dimethylformamide. The mixture was heated to 90° C. for 20 mins then cooled. A solution of 1 mg of tetrakis(triphenylphospine)palladium (0) and 11 mg of borane dimethylamine in 500 μL of dimethylformamide. The mixture was agitated for 20 mins then cooled. A solution of 30 mg of N,N,N′,N′-Tetramethyl-O—(N-succinimidyl)uronium tetrafluoroborate and 20 μL of diisopropylethylamine in 1 mL of dimethylformamide was added. The mixture was agitated for 20 mins then cooled.

### Purification of the Synthesized 2-[(Tert-Butoxycarbonyl)Amino]-4-[¹⁸F]fluorobenzoate

The solution was diluted with 15 mL of water and trapped onto an HLB cartridge. The cartridge was washed with 20% acetonitrile in water. The radiolabeled compound was eluted from the cartridge with acetonitrile.

### Radio Ligation of a Solid Supported RAGAYYVSSYRPGAW (SEQ ID NO:3) Peptide

A solution of the 2-[(tert-butoxycarbonyl)amino]-4-[¹⁸F]fluorobenzoate in N-methyl pyrrolidine was added to the solid supported RAGAYYVSSYRPGAW (SEQ ID NO:3) peptide and agitated for 20 minutes. The solution was drained from the resin. The resin was washed with dimethylformamide, dichloromethane, and ethanol. A solution of trifluoroacetic acid, triisopropyl silane, and water was added to the resin and agitated for 20 minutes. The solution was drained from the resin. The resin was washed with ethanol and pH 4 buffer and dried with air. A solution of phosphate buffered saline (pH 7.4) was added to the resin and agitated for 20 minutes. The solution was collected and analyzed via HPLC to yield the radiolabeled compound in >95% purity with a retention time of 14.9 min.

### Synthesis and Purification of the Cold Standard of the RAGAYYVSSYRPGAW (SEQ ID NO:3) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 14.9 min). The standard was identified by mass spectrometry (calculated for (M+H⁺) 1934.9023; measured 1934.9003).

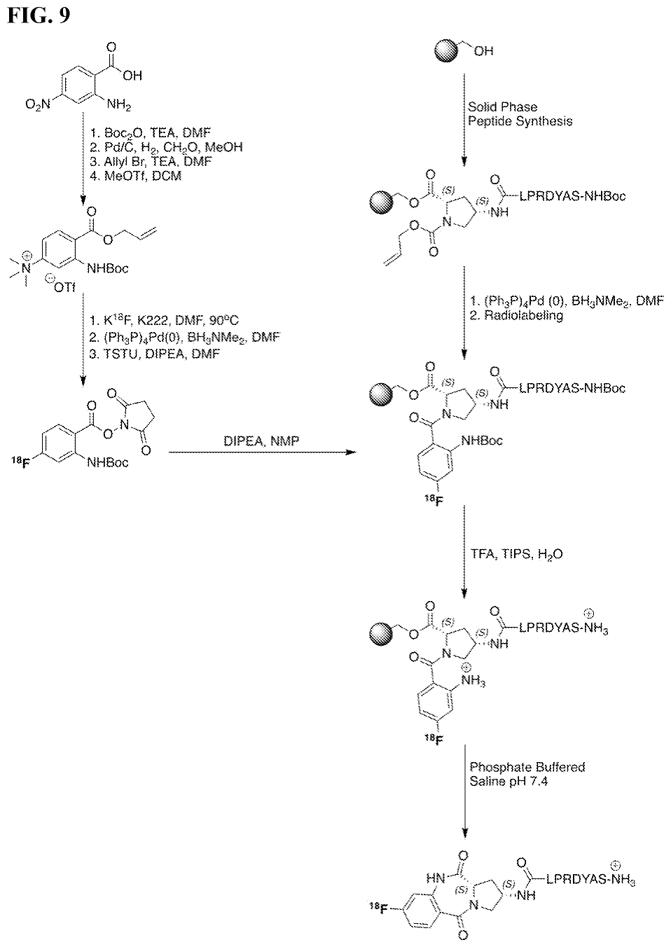

### Example 4: Synthesis of a Solid Supported LPRDYAS (SEQ ID NO:4) Peptide (FIG. 9)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling of allyl 2-[(tert-butoxycarbonyl)amino]-4-trimethylaminobenzoate

A MYJA anion exchange column was conditioned with 6 mL of ethanol followed by 6 mL of potassium bicarbonate followed by 6 mL of water. 500 MBq of an aqueous [¹⁸F]fluoride solution was then passed through the cartridge to immobilized the [¹⁸F]fluoride. The [¹⁸F]fluoride was eluted with a mixture of 1 mg potassium bicarbonate 11 mg of Kryptofix 2.2.2 in 800 μL acetonitrile and 100 μL water. The solution was dried under vacuum and at 115° C. 5 mg of allyl 2-[(tert-butoxycarbonyl)amino]-4-trimethylaminobenzoate was added in 800 μL of dimethylformamide. The mixture was heated to 90° C. for 20 mins then cooled. A solution of 1 mg of tetrakis(triphenylphospine)palladium (0) and 11 mg of borane dimethylamine in 500 μL of dimethylformamide. The mixture was agitated for 20 mins then cooled. A solution of 30 mg of N,N,N′,N′-Tetramethyl-O—(N-succinimidyl)uronium tetrafluoroborate and 20 μL of diisopropylethylamine in 1 mL of dimethylformamide was added. The mixture was agitated for 20 mins then cooled.

### Purification of the Synthesized 2-[(tert-butoxycarbonyl)amino]-4-[¹⁸F]fluorobenzoate

The solution was diluted with 15 mL of water and trapped onto an HLB cartridge. The cartridge was washed with 20% acetonitrile in water. The radiolabeled compound was eluted from the cartridge with acetonitrile.

### Radio Ligation of a Solid Supported LPRDYAS (SEQ ID NO:4) Peptide

A solution of the 2-[(tert-butoxycarbonyl)amino]-4-[¹⁸F]fluorobenzoate in N-methyl pyrrolidine was added to the solid supported LPRDYAS (SEQ ID NO:4) peptide and agitated for 20 minutes. The solution was drained from the resin. The resin was washed with dimethylformamide, dichloromethane, and ethanol. A solution of trifluoroacetic acid, triisopropyl silane, and water was added to the resin and agitated for 20 minutes. The solution was drained from the resin. The resin was washed with ethanol and pH 4 buffer and dried with air. A solution of phosphate buffered saline (pH 7.4) was added to the resin and agitated for 20 minutes. The solution was collected and analyzed via HPLC to yield the radiolabeled compound in >95% purity with a retention time of 5.7 min.

### Synthesis and Purification of the Cold Standard of the LPRDYAS (SEQ ID NO:4) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 5.7 min). The standard was identified by mass spectrometry (calculated for (M+H⁺) 1052.4893; measured 1052.4877).

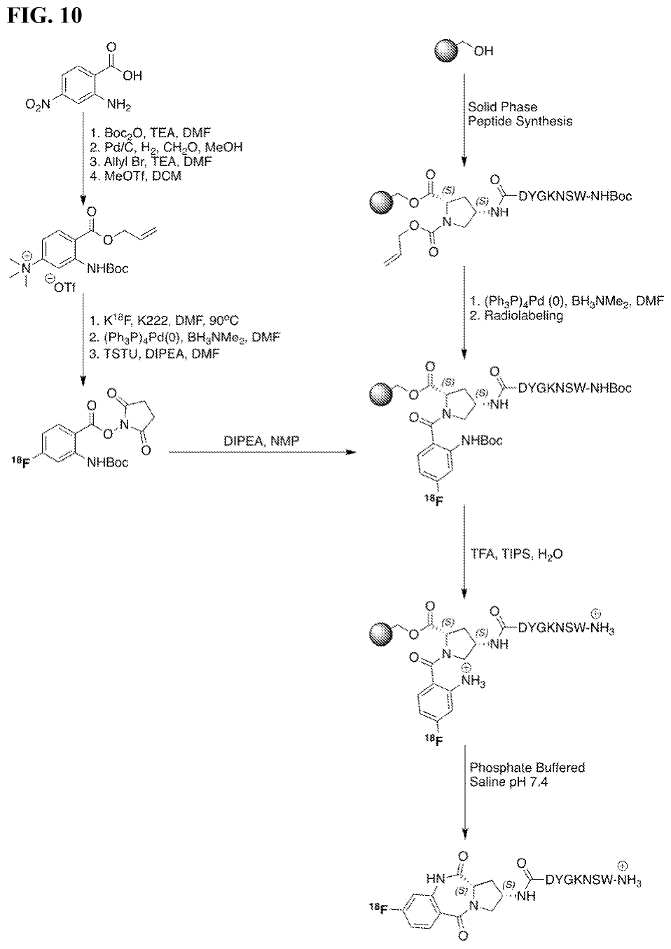

### Example 5: Synthesis of a Solid Supported DYGKNSW (SEQ ID NO:5) Peptide (FIG. 10)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling of allyl 2-[(tert-butoxycarbonyl)amino]-4-trimethylaminobenzoate

A MYJA anion exchange column was conditioned with 6 mL of ethanol followed by 6 mL of potassium bicarbonate followed by 6 mL of water. 500 MBq of an aqueous [¹⁸F]fluoride solution was then passed through the cartridge to immobilized the [¹⁸F]fluoride. The [¹⁸F]fluoride was eluted with a mixture of 1 mg potassium bicarbonate 11 mg of Kryptofix 2.2.2 in 800 μL acetonitrile and 100 μL water. The solution was dried under vacuum and at 115° C. 5 mg of allyl 2-[(tert-butoxycarbonyl)amino]-4-trimethylaminobenzoate was added in 800 μL of dimethylformamide. The mixture was heated to 90° C. for 20 mins then cooled. A solution of 1 mg of tetrakis(triphenylphospine)palladium (0) and 11 mg of borane dimethylamine in 500 μL of dimethylformamide. The mixture was agitated for 20 mins then cooled. A solution of 30 mg of N,N,N′,N′-Tetramethyl-O—(N-succinimidyl)uronium tetrafluoroborate and 20 μL of diisopropylethylamine in 1 mL of dimethylformamide was added. The mixture was agitated for 20 mins then cooled.

### Purification of the Synthesized 2-[(tert-butoxycarbonyl)amino]-4-[¹⁸F]fluorobenzoate

The solution was diluted with 15 mL of water and trapped onto an HLB cartridge. The cartridge was washed with 20% acetonitrile in water. The radiolabeled compound was eluted from the cartridge with acetonitrile.

### Radio Ligation of a Solid Supported DYGKNSW (SEQ ID NO:5) Peptide

A solution of the 2-[(tert-butoxycarbonyl)amino]-4-[¹⁸F]fluorobenzoate in N-methyl pyrrolidine was added to the solid supported DYGKNSW (SEQ ID NO:5) peptide and agitated for 20 minutes. The solution was drained from the resin. The resin was washed with dimethylformamide, dichloromethane, and ethanol. A solution of trifluoroacetic acid, triisopropyl silane, and water was added to the resin and agitated for 20 minutes. The solution was drained from the resin. The resin was washed with ethanol and pH 4 buffer and dried with air. A solution of phosphate buffered saline (pH 7.4) was added to the resin and agitated for 20 minutes. The solution was collected and analyzed via HPLC to yield the radiolabeled compound in >95% purity with a retention time of 6.8 min.

### Synthesis and Purification of the Cold Standard of the DYGKNSW (SEQ ID NO:5) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 6.8 min). The standard was identified by mass spectrometry (calculated for (M+H⁺) 1100.4523; measured 1100.4512).

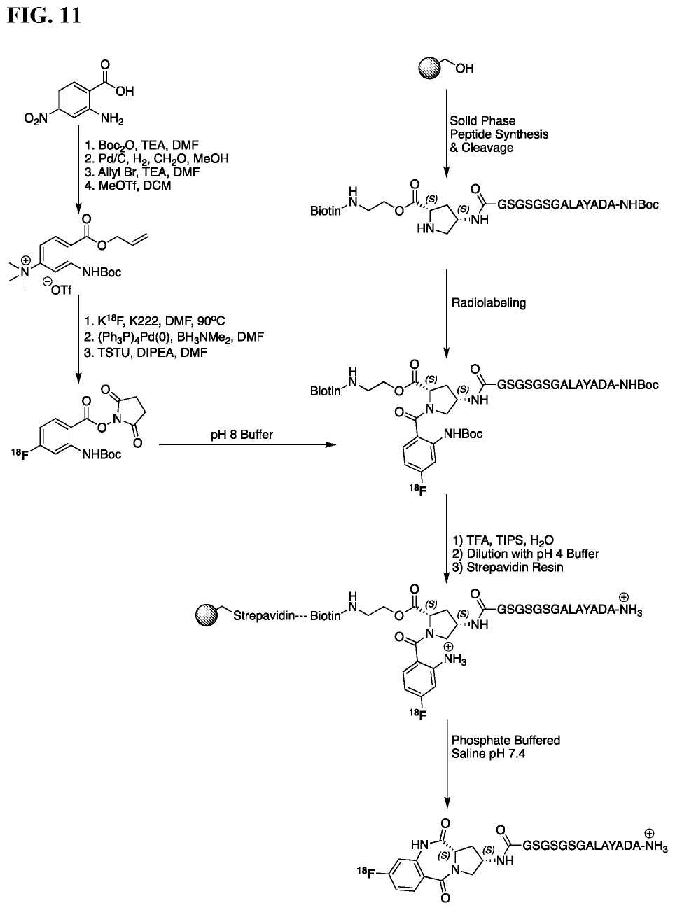

### Example 6: Synthesis of a Biotin GSGSGSGALAYADA (SEQ ID NO:1) Peptide (FIG. 11)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling of allyl 2-[(tert-butoxycarbonyl)amino]-4-trimethylaminobenzoate

A MYJA anion exchange column was conditioned with 6 mL of ethanol followed by 6 mL of potassium bicarbonate followed by 6 mL of water. 500 MBq of an aqueous [¹⁸F]fluoride solution was then passed through the cartridge to immobilized the [¹⁸F]fluoride. The [¹⁸F]fluoride was eluted with a mixture of 1 mg potassium bicarbonate 11 mg of Kryptofix 2.2.2 in 800 μL acetonitrile and 100 μL water. The solution was dried under vacuum and at 115° C. 5 mg of allyl 2-[(tert-butoxycarbonyl)amino]-4-trimethylaminobenzoate was added in 800 μL of dimethylformamide. The mixture was heated to 90° C. for 20 mins then cooled. A solution of 1 mg of tetrakis(triphenylphospine)palladium (0) and 11 mg of borane dimethylamine in 500 μL of dimethylformamide. The mixture was agitated for 20 mins then cooled. A solution of 30 mg of N,N,N′,N′-Tetramethyl-O—(N-succinimidyl)uronium tetrafluoroborate and 20 μL of diisopropylethylamine in 1 mL of dimethylformamide was added. The mixture was agitated for 20 mins then cooled.

### Purification of the Synthesized 2-[(tert-butoxycarbonyl)amino]-4-[¹⁸F]fluorobenzoate

The solution was diluted with 15 mL of water and trapped onto an HLB cartridge. The cartridge was washed with 20% acetonitrile in water. The radiolabeled compound was eluted from the cartridge with acetonitrile.

### Radio Ligation of a Biotin GSGSGSGALAYADA (SEQ ID NO:1) Peptide

A solution of the 2-[(tert-butoxycarbonyl)amino]-4-[¹⁸F]fluorobenzoate in pH 8 Buffer was added to the biotin GSGSGSGALAYADA (SEQ ID NO:1) peptide and agitated for 20 minutes. A solution of trifluoroacetic acid, triisopropyl silane, and water was added and agitated for 20 minutes. The solution was diluted 100× with pH 4 buffer and the solution was passed through a streptavidin resin cartridge. The cartridge was washed with pH 4 buffer. A solution of phosphate buffered saline (pH 7.4) was slowly added to the cartridge (10 L/min). The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 8.4 min). The standard was identified by mass spectrometry (calculated for (M+H⁺) 1414.5963; measured 1414.5957).

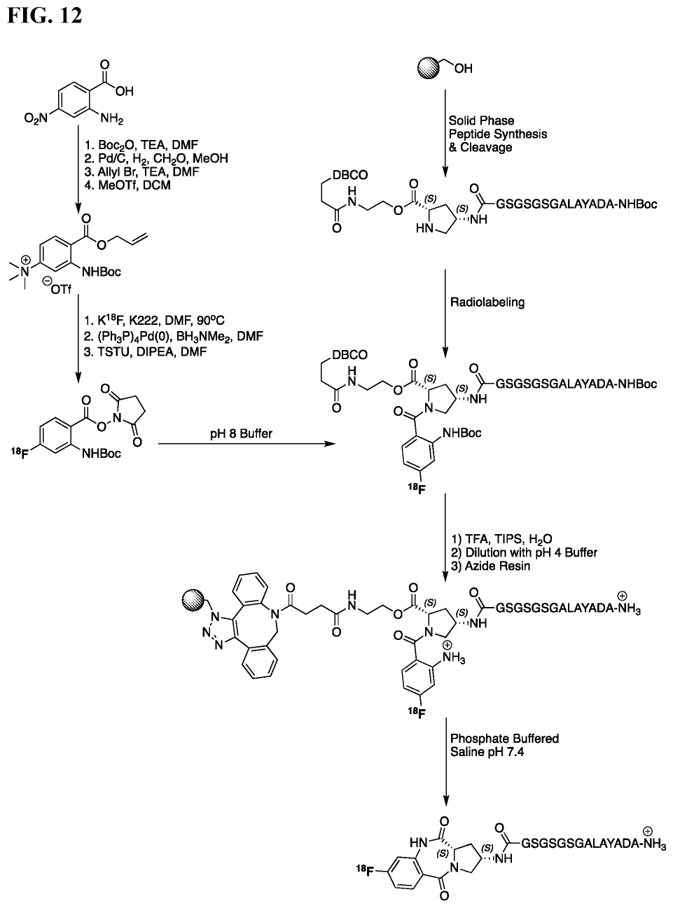

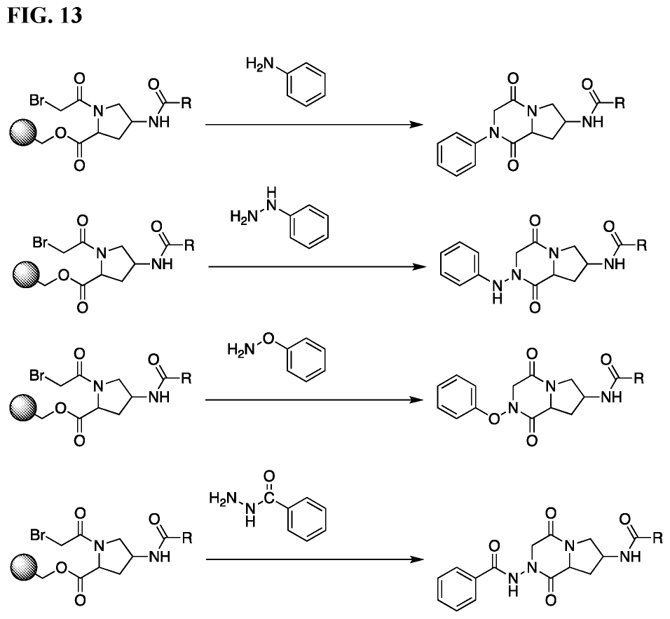

### Example 7: Synthesis of a DBCO GSGSGSGALAYADA (SEQ ID NO:1) Peptide (FIG. 12)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling of allyl 2-[(tert-butoxycarbonyl)amino]-4-trimethylaminobenzoate

A MYJA anion exchange column was conditioned with 6 mL of ethanol followed by 6 mL of potassium bicarbonate followed by 6 mL of water. 500 MBq of an aqueous [¹⁸F]fluoride solution was then passed through the cartridge to immobilized the [¹⁸F]fluoride. The [¹⁸F]fluoride was eluted with a mixture of 1 mg potassium bicarbonate 11 mg of Kryptofix 2.2.2 in 800 μL acetonitrile and 100 μL water. The solution was dried under vacuum and at 115° C. 5 mg of allyl 2-[(tert-butoxycarbonyl)amino]-4-trimethylaminobenzoate was added in 800 μL of dimethylformamide. The mixture was heated to 90° C. for 20 mins then cooled. A solution of 1 mg of tetrakis(triphenylphospine)palladium (0) and 11 mg of borane dimethylamine in 500 μL of dimethylformamide. The mixture was agitated for 20 mins then cooled. A solution of 30 mg of N,N,N′,N′-Tetramethyl-O—(N-succinimidyl)uronium tetrafluoroborate and 20 μL of diisopropylethylamine in 1 mL of dimethylformamide was added. The mixture was agitated for 20 mins then cooled.

### Purification of the synthesized 2-[(tert-butoxycarbonyl)amino]-4-[¹⁸F]fluorobenzoate

The solution was diluted with 15 mL of water and trapped onto an HLB cartridge. The cartridge was washed with 20% acetonitrile in water. The radiolabeled compound was eluted from the cartridge with acetonitrile.

### Radio Ligation of a DBCO GSGSGSGALAYADA (SEQ ID NO:1) Peptide

A solution of the 2-[(tert-butoxycarbonyl)amino]-4-[¹⁸F]fluorobenzoate in pH 8 Buffer was added to the DBCO GSGSGSGALAYADA (SEQ ID NO:1) peptide and agitated for 20 minutes. A solution of trifluoroacetic acid, triisopropyl silane, and water was added and agitated for 20 minutes. The solution was diluted 100× with pH 4 buffer and the solution was passed through an azide resin cartridge. The cartridge was washed with pH 4 buffer. A solution of phosphate buffered saline (pH 7.4) was slowly added to the cartridge (10 μL/min). The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 8.4 min). The standard was identified by mass spectrometry (calculated for (M+H⁺) 1414.5963; measured 1414.5957).

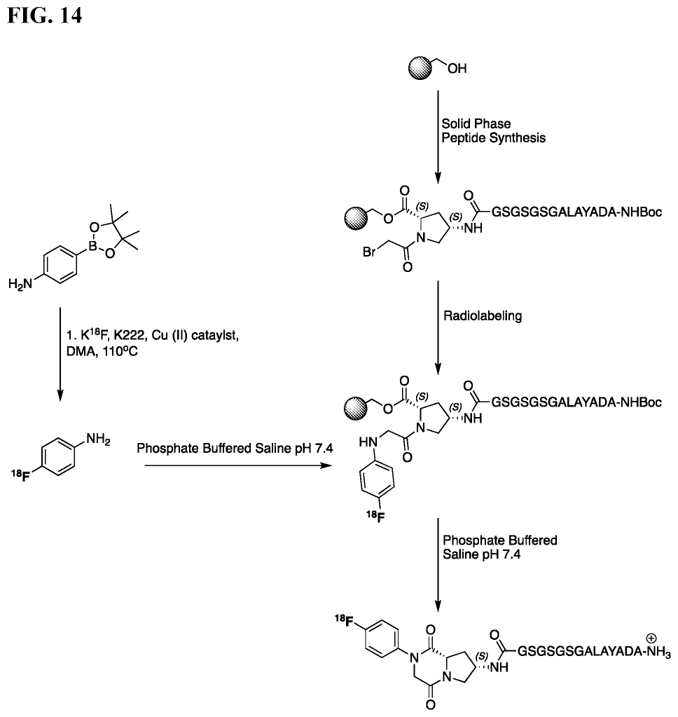

### Example 8: Synthesis of a Solid Supported GSGSGSGALAYADA (SEQ ID NO:1) Peptide (FIG. 14)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported GSGSGSGALAYADA (SEQ ID NO:1) Peptide

A solution of the 4-[¹⁸F]aniline in phosphate buffered saline pH 7.4 was added to the solid supported GSGSGSGALAYADA peptide and agitated for 20 minutes. The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 4-fluoroaniline. HPLC purification was used to purify the standard (retention time of 8.4 min). The standard was identified by mass spectrometry (calculated for (M+H) 1428.6120; measured 1428.6111).

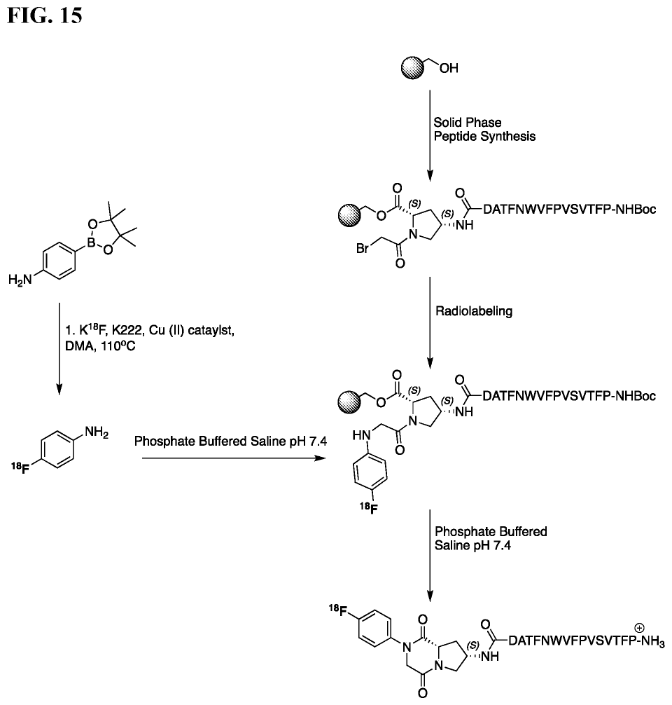

### Example 9: Synthesis of a Solid Supported DATFNWVFPVSVTFP (SEQ ID NO:2) Peptide (FIG. 15)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported DATFNWVFPVSVTFP (SEQ ID NO:2) Peptide

A solution of the 4-[¹⁸F]aniline in phosphate buffered saline pH 7.4 was added to the solid supported DATFNWVFPVSVTFP (SEQ ID NO:2) peptide and agitated for 20 minutes. The solution was collected and analyzed via HPLC to yield the radiolabeled compound in >95% purity with a retention time of 23.1 min.

### Synthesis and Purification of the Cold Standard of the DATFNWVFPVSVTFP (SEQ ID NO:2) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 23.1 min). The standard was identified by mass spectrometry (calculated for (M+H⁺) 1971.9370; measured 1971.9357).

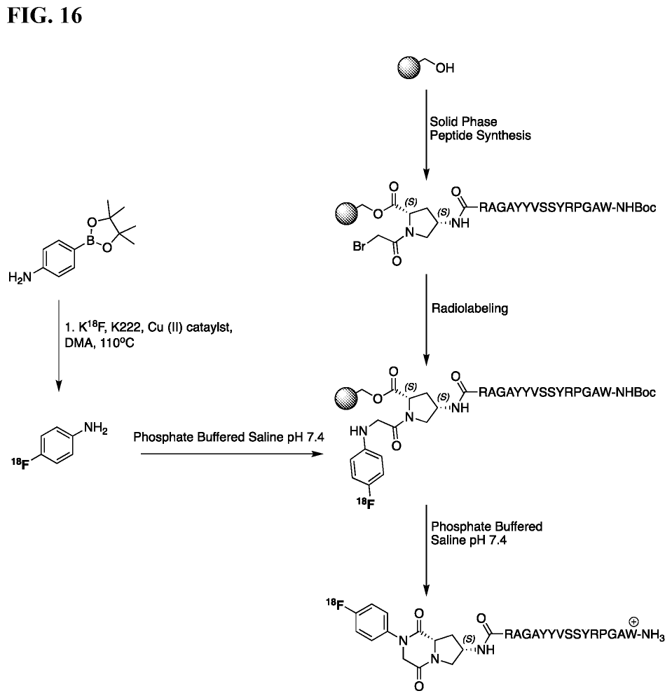

### Example 10: Synthesis of a Solid Supported RAGAYYVSSYRPGAW (SEQ ID NO:3) Peptide (FIG. 16)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported RAGAYYVSSYRPGAW (SEQ ID NO:3) Peptide

A solution of the 4-[¹⁸F]aniline in phosphate buffered saline pH 7.4 was added to the solid supported RAGAYYVSSYRPGAW (SEQ ID NO:3) peptide and agitated for 20 minutes. The solution was collected and analyzed via HPLC to yield the radiolabeled compound in >95% purity with a retention time of 14.9 min.

### Synthesis and Purification of the Cold Standard of the RAGAYYVSSYRPGAW (SEQ ID NO:3) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 14.9 min). The standard was identified by mass spectrometry (calculated for (M+H⁺) 1948.9180; measured 1948.9161).

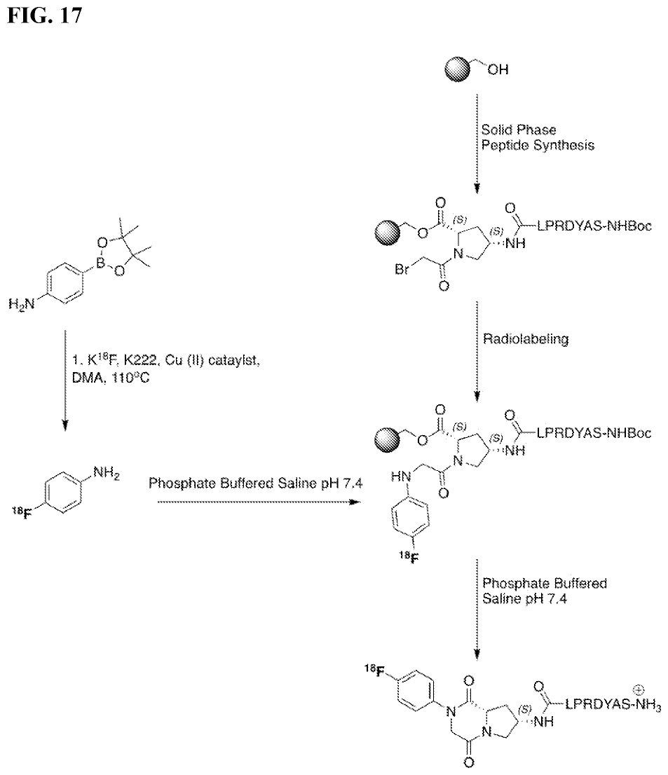

### Example 11: Synthesis of a Solid Supported LPRDYAS (SEQ ID NO:4) Peptide (FIG. 17)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported LPRDYAS (SEQ ID NO:4) Peptide

A solution of the 4-[¹⁸F]aniline in phosphate buffered saline pH 7.4 was added to the solid supported LPRDYAS (SEQ ID NO:4) peptide and agitated for 20 minutes. The solution was collected and analyzed via HPLC to yield the radiolabeled compound in >95% purity with a retention time of 5.7 min.

### Synthesis and Purification of the Cold Standard of the LPRDYAS (SEQ ID NO:4) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 5.7 min). The standard was identified by mass spectrometry (calculated for (M+H⁺) 1066.5050; measured 1066.5038).

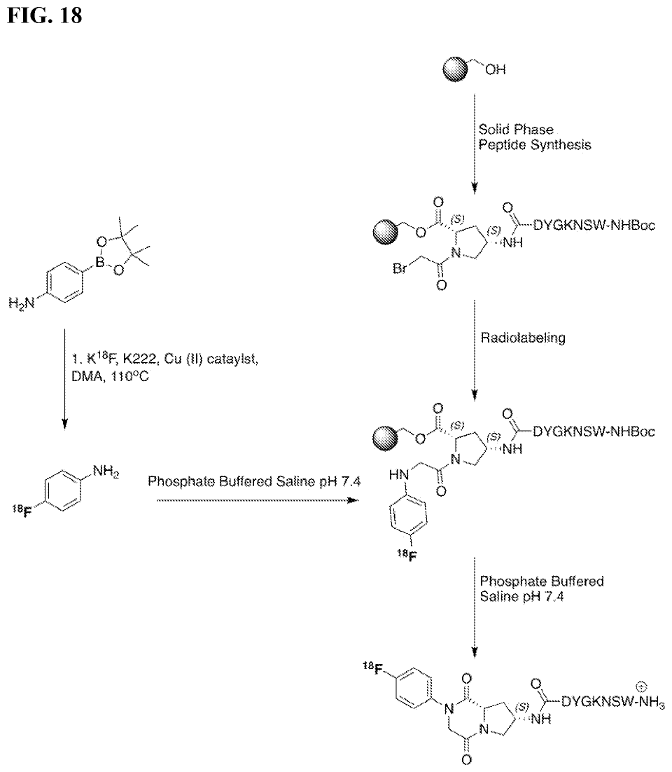

### Example 12: Synthesis of a Solid Supported DYGKNSW (SEQ ID NO:5) Peptide (FIG. 18)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported DYGKNSW (SEQ ID NO:5) Peptide

A solution of the 4-[¹⁸F]aniline in phosphate buffered saline pH 7.4 was added to the solid supported DYGKNSW (SEQ ID NO:5) peptide and agitated for 20 minutes. The solution was collected and analyzed via HPLC to yield the radiolabeled compound in >95% purity with a retention time of 6.8 min.

### Synthesis and Purification of the Cold Standard of the DYGKNSW (SEQ ID NO:5) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 6.8 min). The standard was identified by mass spectrometry (calculated for (M+H⁺) 1114.4680; measured 1114.4670).

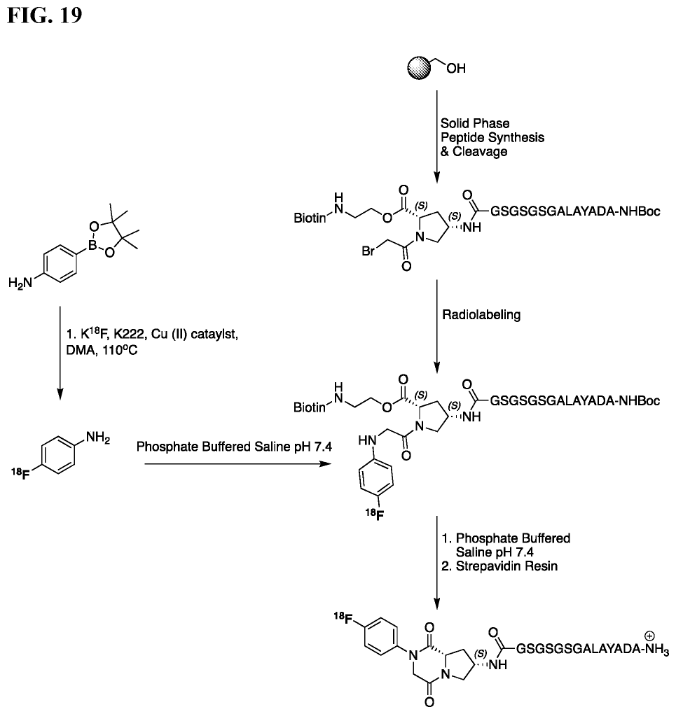

### Example 13: Synthesis of a Biotin GSGSGSGALAYADA (SEQ ID NO:1) Peptide (FIG. 19)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Biotin GSGSGSGALAYADA (SEQ ID NO:1) Peptide

A solution of the 4-[¹⁸F]aniline in phosphate buffered saline pH 7.4 was added to the biotin GSGSGSGALAYADA (SEQ ID NO:1) peptide and agitated for 20 minutes. The solution was slowly passed through a streptavidin resin cartridge (10 μL/min). The solution was collected and analyzed via HPLC to yield the radiolabeled compound in >95% purity with a retention time of 8.4 min.

### Synthesis and Purification of the Cold Standard of the GSGSGSGALAYADA (SEQ I^{D} NO:1) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 8.4 min). The standard was identified by mass spectrometry (calculated for (M+H⁺) 1428.6120; measured 1428.6111).

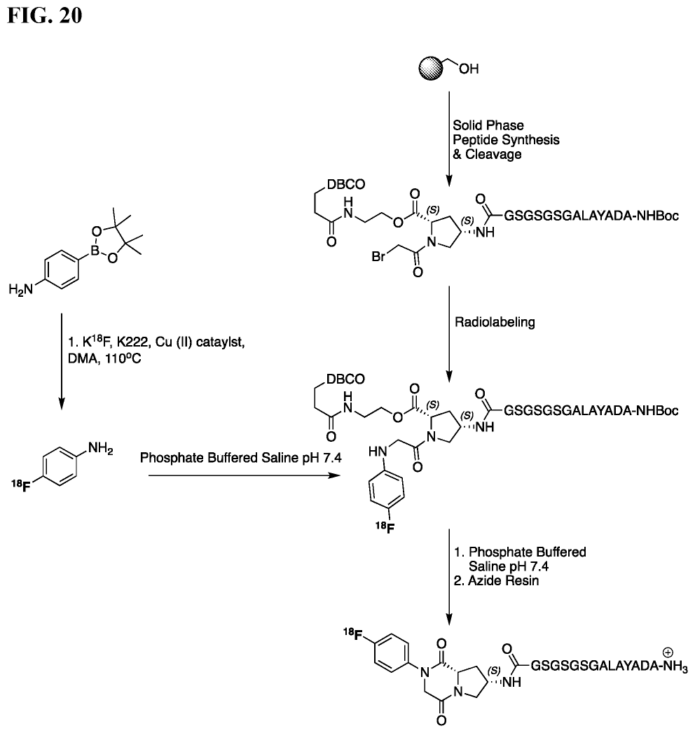

### Example 14: Synthesis of a DBCO GSGSGSGALAYADA (SEQ ID NO:1) Peptide (FIG. 20)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation of a DBCO GSGSGSGALAYADA (SEQ ID NO:1) Peptide

A solution of the 4-[¹⁸F]aniline in phosphate buffered saline pH 7.4 was added to the DBCO GSGSGSGALAYADA (SEQ ID NO:1) peptide and agitated for 20 minutes. The solution was slowly passed through an azide resin cartridge (10 μL/min). The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 8.4 min). The standard was identified by mass spectrometry (calculated for (M+H⁺) 1428.6120; measured 1428.6111).

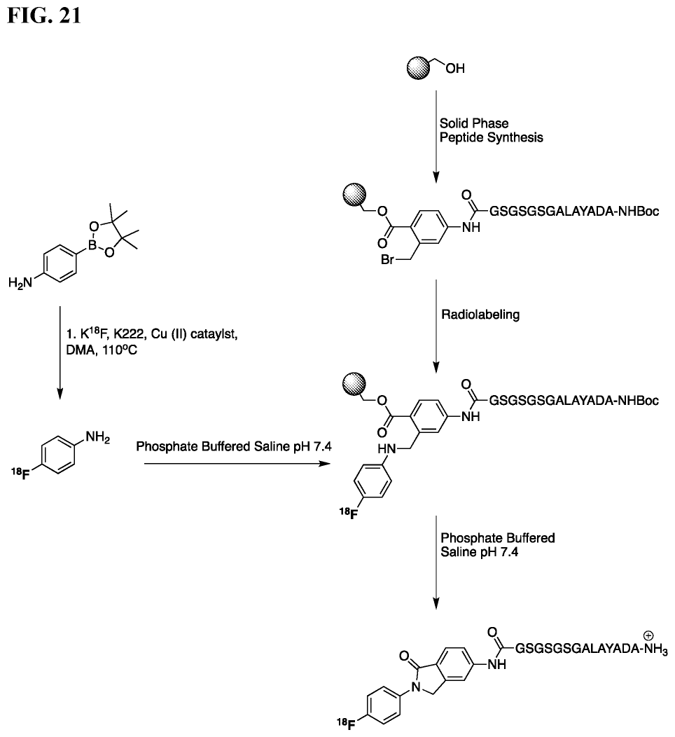

### Example 15: Synthesis of a Solid Supported GSGSGSGALAYADA (SEQ ID NO:1) Peptide (FIG. 21)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported GSGSGSGALAYADA (SEQ ID NO:1) Peptide

A solution of the 4-[¹⁸F]aniline in phosphate buffered saline pH 7.4 was added to the solid supported GSGSGSGALAYADA (SEQ ID NO:1) peptide and agitated for 20 minutes. The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 4-fluoroaniline. HPLC purification was used to purify the standard (retention time of 8.4 min). The standard was identified by mass spectrometry (calculated for (M+H) 1407.5905; measured 1407.5911).

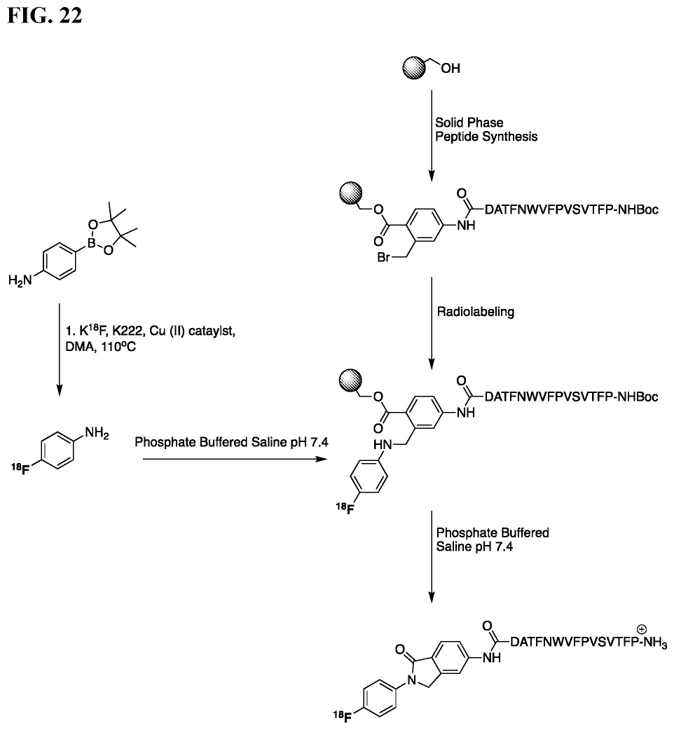

### Example 16: Synthesis of a Solid Supported DATFNWVFPVSVTFP (SEQ ID NO:2) Peptide (FIG. 22)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported DATFNWVFPVSVTFP (SEQ ID NO:2) Peptide

A solution of the 4-[¹⁸F]aniline in phosphate buffered saline pH 7.4 was added to the solid supported DATFNWVFPVSVTFP (SEQ ID NO:2) peptide and agitated for 20 minutes. The solution was collected and analyzed via HPLC to yield the radiolabeled compound in >95% purity with a retention time of 23.1 min.

### Synthesis and Purification of the Cold Standard of the DATFNWVFPVSVTFP (SEQ ID NO:2) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 23.1 min). The standard was identified by mass spectrometry (calculated for (M+H⁺) 1950.9155; measured 1950.9159).

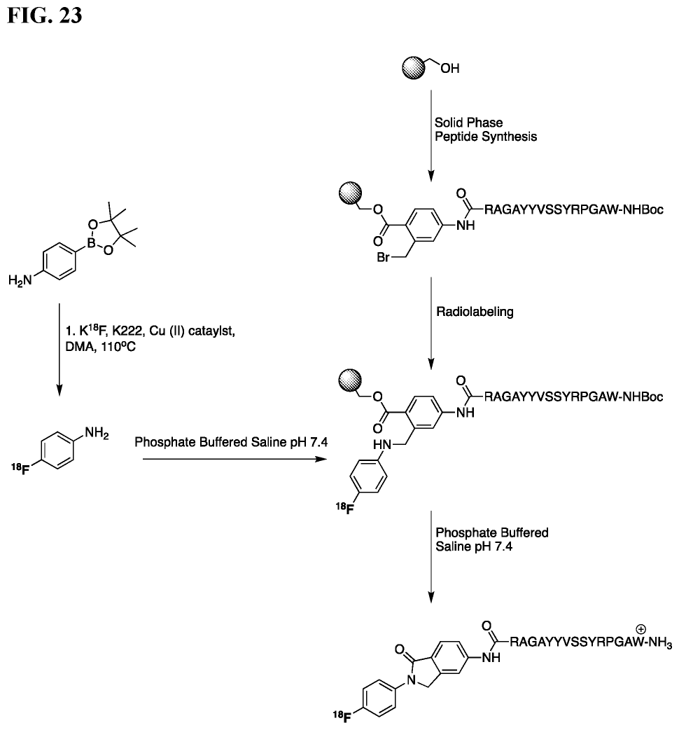

### Example 17: Synthesis of a Solid Supported RAGAYYVSSYRPGAW (SEQ ID NO:3) Peptide (FIG. 23)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported RAGAYYVSSYRPGAW (SEQ ID NO:3) Peptide

A solution of the 4-[¹⁸F]aniline in phosphate buffered saline pH 7.4 was added to the solid supported RAGAYYVSSYRPGAW (SEQ ID NO:3) peptide and agitated for 20 minutes. The solution was collected and analyzed via HPLC to yield the radiolabeled compound in >95% purity with a retention time of 14.9 min.

### Synthesis and Purification of the Cold Standard of the RAGAYYVSSYRPGAW (SEQ ID NO:3) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 14.9 min). The standard was identified by mass spectrometry (calculated for (M+H⁺) 1927.8965; measured 1927.8985).

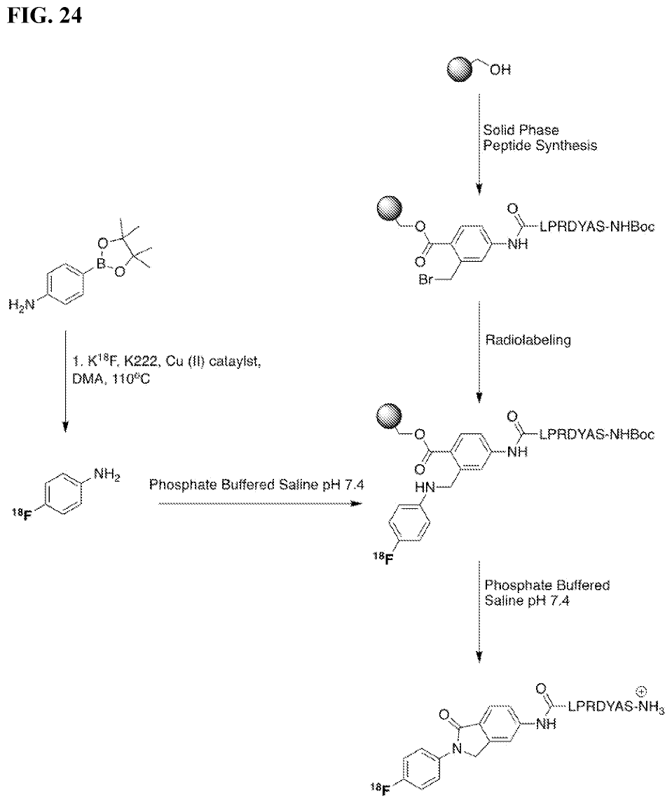

### Example 18: Synthesis of a Solid Supported LPRDYAS (SEQ ID NO:4) Peptide (FIG. 24)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported LPRDYAS (SEQ ID NO:4) Peptide

A solution of the 4-[¹⁸F]aniline in phosphate buffered saline pH 7.4 was added to the solid supported LPRDYAS (SEQ ID NO:4) peptide and agitated for 20 minutes. The solution was collected and analyzed via HPLC to yield the radiolabeled compound in >95% purity with a retention time of 5.7 min.

### Synthesis and Purification of the Cold Standard of the LPRDYAS (SEQ ID NO:4) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 5.7 min). The standard was identified by mass spectrometry (calculated for (M+H⁺) 1045.4835; measured 1045.4851).

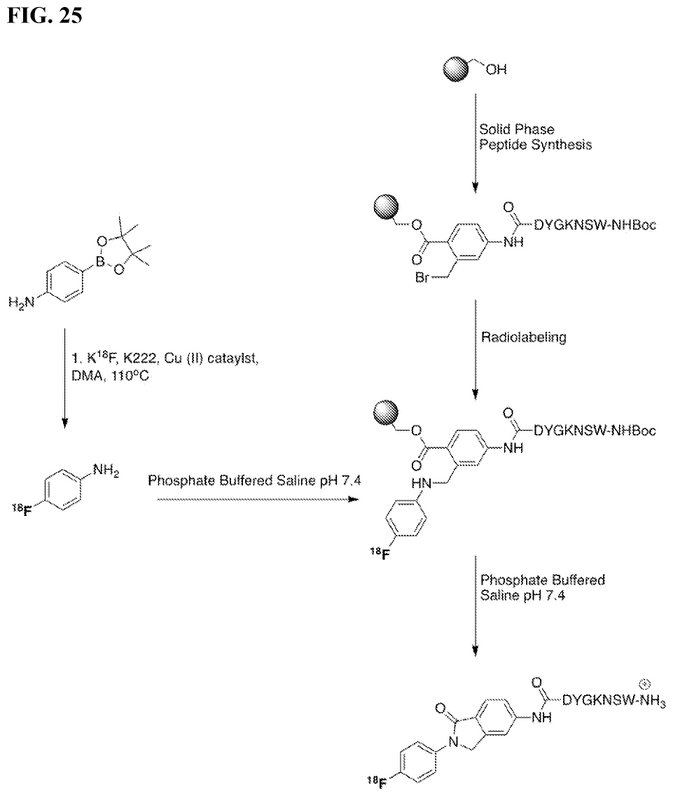

### Example 19: Synthesis of a Solid Supported DYGKNSW (SEQ ID NO:5) Peptide (FIG. 25)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Solid Supported DYGKNSW (SEQ ID NO:5) Peptide

A solution of the 4-[¹⁸F]aniline in phosphate buffered saline pH 7.4 was added to the solid supported DYGKNSW (SEQ ID NO:5) peptide and agitated for 20 minutes. The solution was collected and analyzed via HPLC to yield the radiolabeled compound in >95% purity with a retention time of 6.8 min.

### Synthesis and Purification of the Cold Standard of the DYGKNSW (SEQ ID NO:5) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 6.8 min). The standard was identified by mass spectrometry (calculated for (M+H⁺) 1093.4465; measured 1093.4476).

### Example 20: Synthesis of a Biotin GSGSGSGALAYADA (SEQ ID NO:1) Peptide (FIG. 26)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation and Cleavage of a Biotin GSGSGSGALAYADA (SEQ ID NO:1) Peptide

A solution of the 4-[¹⁸F]aniline in phosphate buffered saline pH 7.4 was added to the biotin GSGSGSGALAYADA (SEQ ID NO:1) peptide and agitated for 20 minutes. The solution was slowly passed through a streptavidin resin cartridge (10 μL/min). The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 8.4 min). The standard was identified by mass spectrometry (calculated for (M+H⁺) 1407.5905; measured 1407.5911).

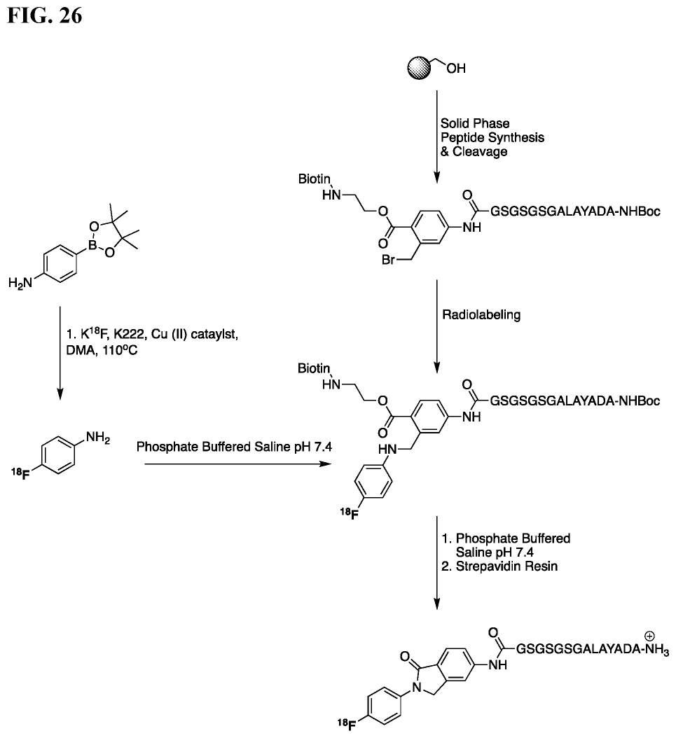

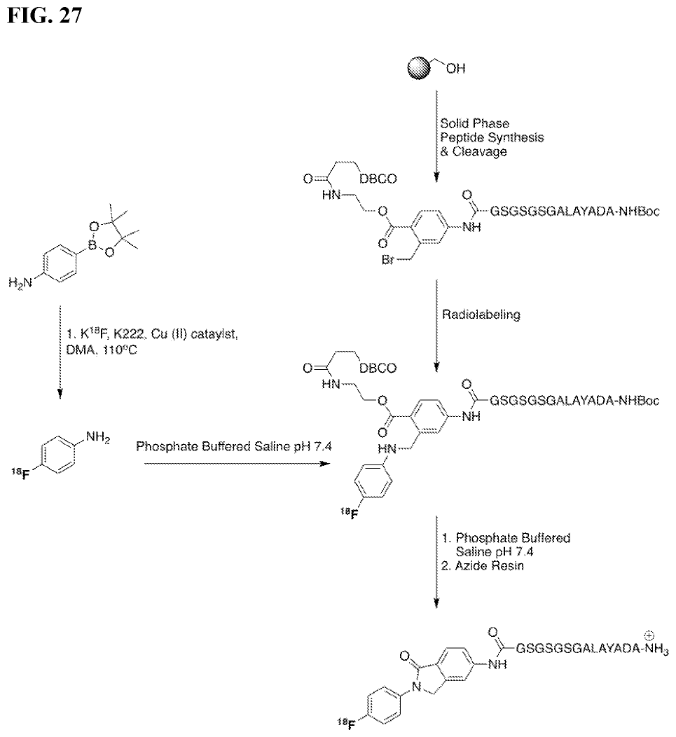

### Example 21: Synthesis of a DBCO GSGSGSGALAYADA (SEQ ID NO:1) Peptide (FIG. 27)

The peptide was assembled using conventional FMOC solid phase synthesis techniques using 10 equivalents of the amino acid, 10 equivalents of (1-[Bis(dimethylamino)methylene]-1H-1,2,3-triazolo[4,5-b]pyridinium 3-oxide hexafluorophosphate, 20 equivalents of diisopropylethylamine in N-methyl-pyrrolidinone (0.3 M). Coupling reactions were run for 1 hour. The FMOC protecting groups were removed with 20% piperidine in dimethylformamide. The deprotection reactions were run for 15 mins and repeated twice. The BOC protecting group was used for the last amino acid in the sequence in place of FMOC. After every coupling and deprotection step, the resin was washed with dimethylformamide, dichloromethane, and methanol.

### Radiolabeling and Purification of Allyl 4-[¹⁸F]aniline

4-[¹⁸F]aniline was performed as previously described by Tredwell et al. (Angew Chem IE, 2014, 126, 30, 7885-7889).

### Radio Ligation of a DBCO GSGSGSGALAYADA (SEQ ID NO:1) Peptide

A solution of the 4-[¹⁸F]aniline in phosphate buffered saline pH 7.4 was added to the DBCO GSGSGSGALAYADA (SEQ ID NO:1) peptide and agitated for 20 minutes. The solution was slowly passed through an azide resin cartridge (10 μL/min). The solution was collected and analyzed via HPLC to yield the radiolabeled compound in >95% purity with a retention time of 8.4 min.

### Synthesis and Purification of the Cold Standard of the GSGSGSGALAYADA (SEQ ID NO:1) Peptide

The standard was synthesized identically to the radiolabeled peptide using [¹⁹F] 2-((tert-butoxycarbonyl)amino)-4-fluorobenzoic acid. HPLC purification was used to purify the standard (retention time of 8.4 min). The standard was identified by mass spectrometry (calculated for (M+H⁺) 1407.5905; measured 1407.5911).

### Radiolabeling of N′-[(tert-butoxy)carbonyl]-4-nitrobenzohydrazide (FIG. 28)

A MYJA anion exchange resin was conditioned with 6 mL of ethanol followed by 6 mL of potassium bicarbonate followed by 6 mL of water. 500 MBq of an aqueous [¹⁸F]fluoride solution was then passed through the cartridge to immobilize the [¹⁸F]fluoride. The [¹⁸F]fluoride was eluted with a mixture of 1 mg of potassium bicarbonate and 11 mg of Kryptofix 2.2.2 in 800 μL of acetonitrile and 100 μL of water. The solution was dried under vacuum at 115° C. 5 mg of N′-[(tert-butoxy)carbonyl]-4-nitrobenzohydrazide was added in 800 μL of dimethylformamide. The mixture was heated to 90° C. for 20 minutes then cooled. An 800 μL of trifluoroacetic acid is then added and the mixture was again heated to 90° C. for 20 minutes then cooled.

### Purification of 4-[¹⁸F]fluorobenzohydrazide

The solution was diluted with 15 mL of water and purified on reverse phase HPLC. The product fractions were collected and diluted with 30 mL of water and trapped onto a C₁₈ cartridge. The cartridge was washed with water and the radiolabeled compound was eluted from the cartridge in ethanol then diluted in phosphate buffered saline.

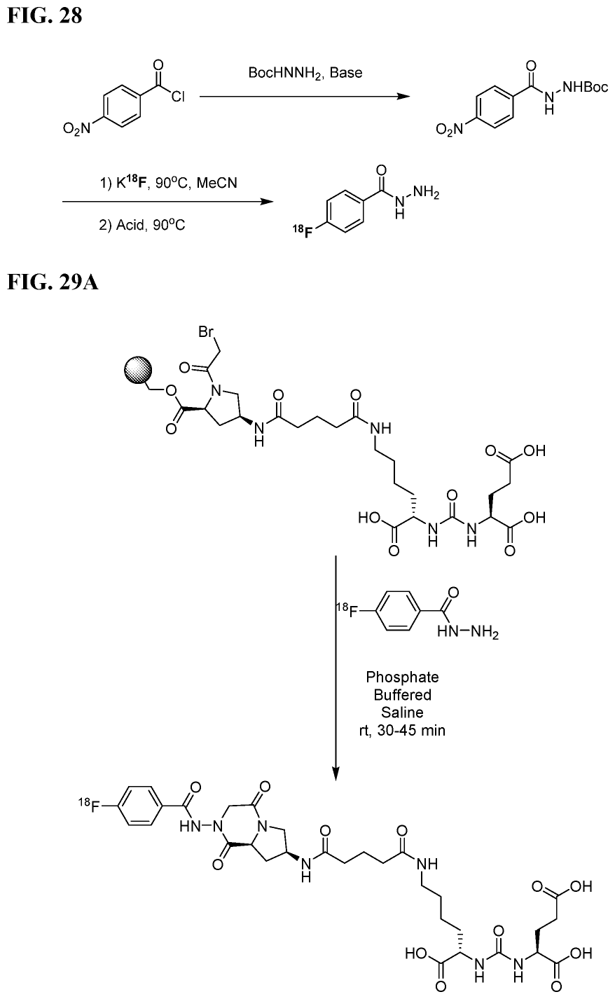

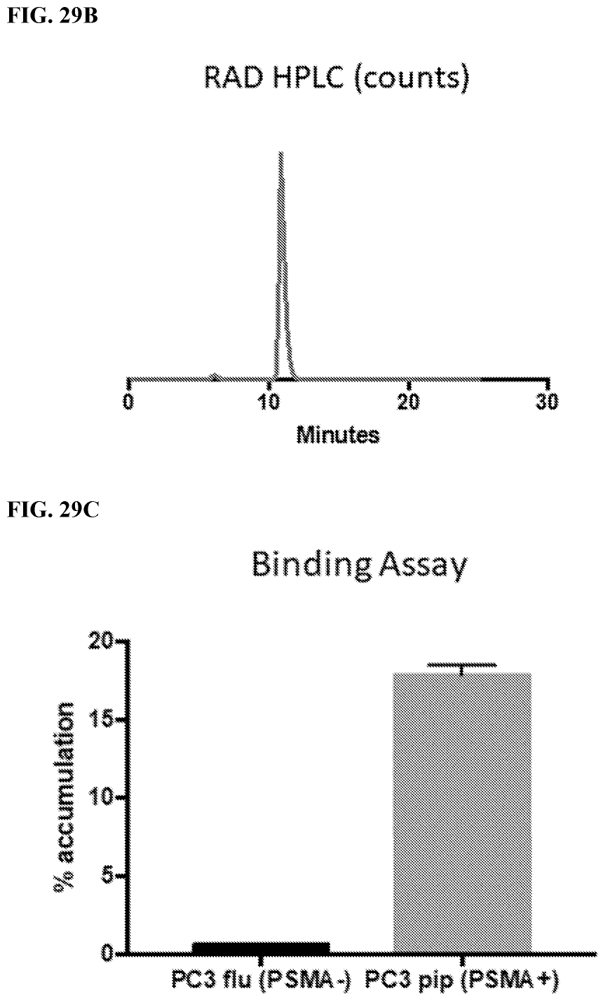

### Radiolabeling of Prostate Specific Membrane Antigen (PSMA) Binding Molecule (FIG. 29A)

A solution of the 4-[¹⁸F]fluorobenzohydrazide in phosphate buffered saline pH 7.4 was added to the solid supported PSMA binding molecule and agitated for 45 minutes. The solution was collected and analyzed via HPLC to yield the radiolabeled compound in >95% purity with a retention time of 10.8 minutes as a single peak by gamma detection.

### Biological Evaluation of Prostate Specific Membrane Antigen (PSMA) Binding Molecule (FIGS. 29B-29C)

The radiolabeled PSMA binding molecule was assayed using a standard cellular uptake assay using PC3 flu cell line (PSMA−) and PC3 pip cell line (PSMA+). The tracer was incubated with the cell for 90 minutes. The solution and cells were separated, washed and analyzed on a gamma counter. The tracer performed consistently to the literature with an observed ˜60-fold difference in uptake.

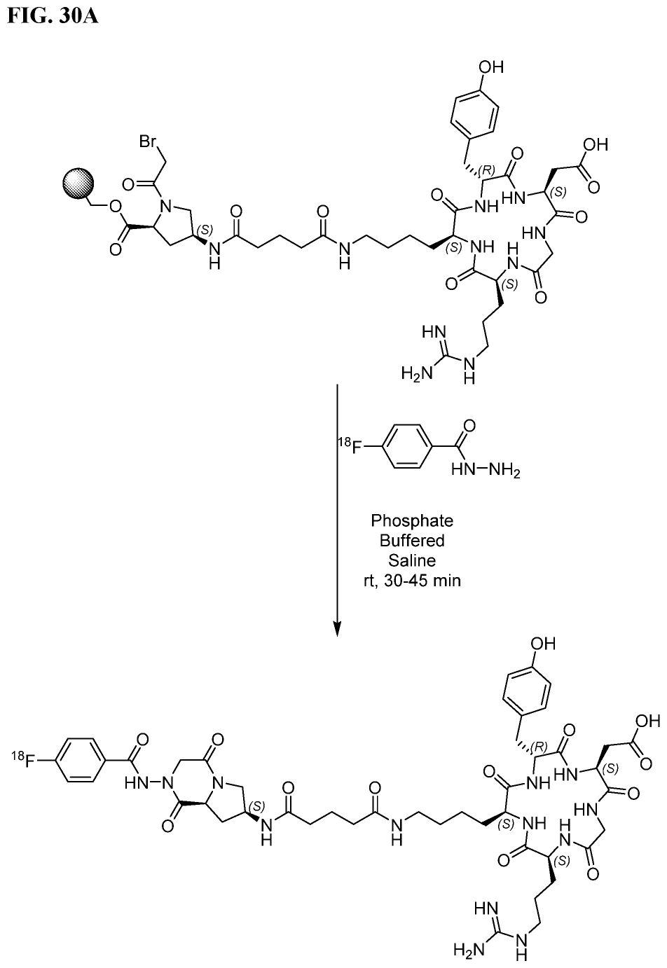

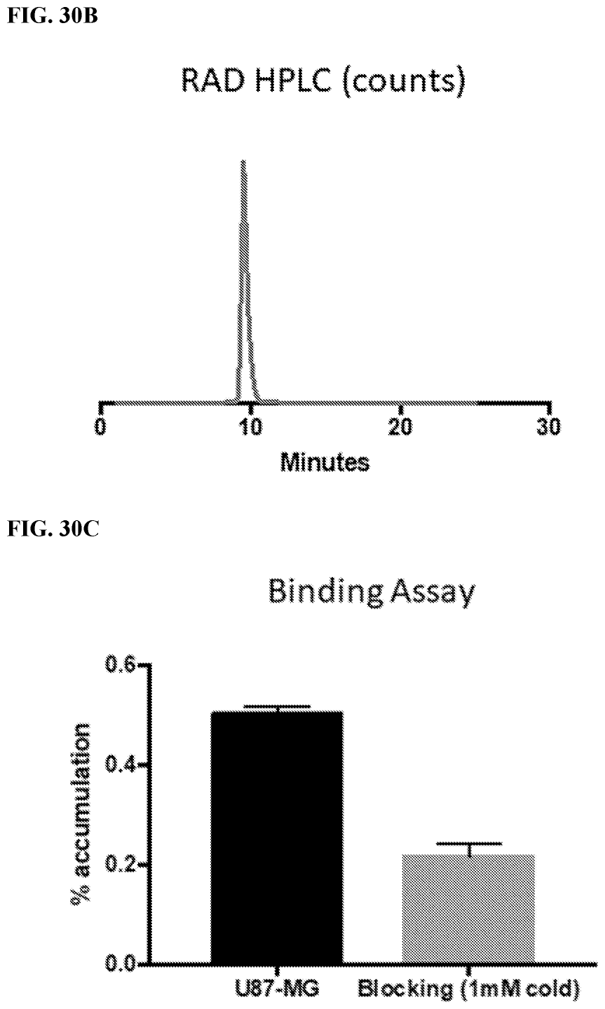

### Radiolabeling of an Integrin Receptor Binding Cyclopeptide (FIG. 30A)

A solution of the 4-[¹⁸F]fluorobenzohydrazide in phosphate buffered saline pH 7.4 was added to the solid supported integrin binding molecule and agitated for 45 minutes. The solution was collected and analyzed via HPLC to yield the radiolabeled compound in >95% purity with a retention time of 9.5 minutes as a single peak by gamma detection.

### Biological Evaluation of an Integrin Receptor Binding Cyclopeptide (FIGS. 30B-30C)

The radiolabeled integrin receptor binding molecule was assayed using a standard cellular blocking assay using U87-MG cell line in the presence and absence of 1 mM of the “cold” molecule. The tracer was incubated with the cell for 90 minutes. The solution and cells were separated, washed and analyzed on a gamma counter. The tracer performed consistently to the literature with an observed ˜60% blockage of uptake.

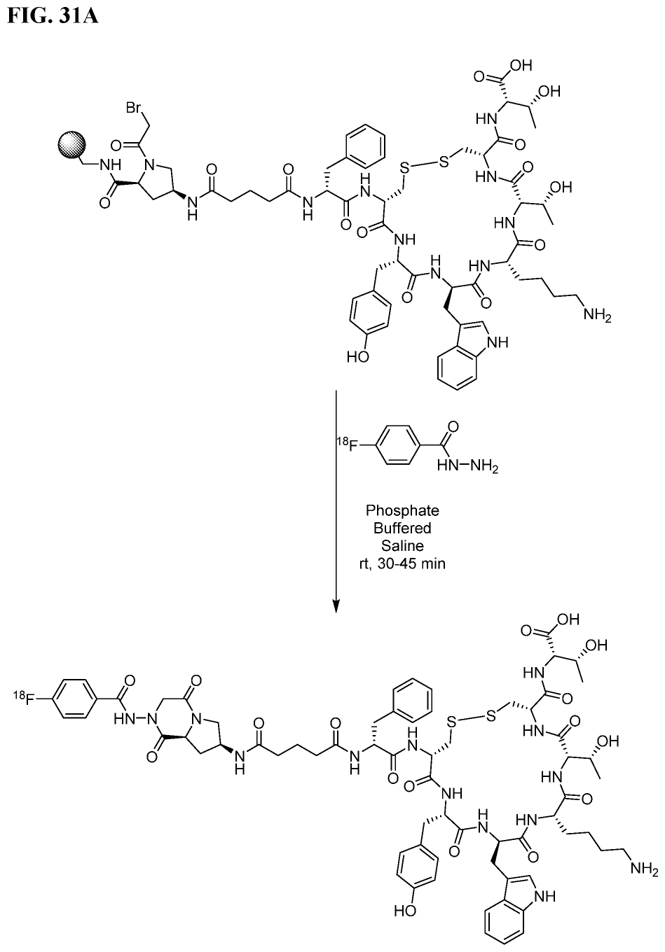

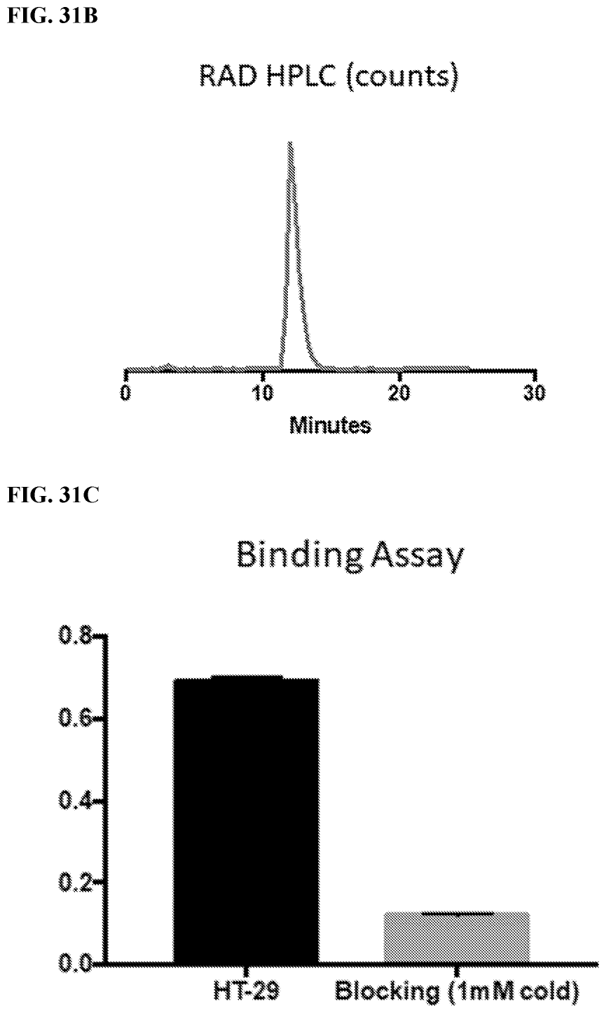

### Radiolabeling of a Somatostatin Receptor Binding Cyclopeptide (FIG. 31A)

A solution of the 4-[¹⁸F]fluorobenzohydrazide in phosphate buffered saline pH 7.4 was added to the solid supported somatostatin binding molecule and agitated for 45 minutes. The solution was collected and analyzed via HPLC to yield the radiolabeled compound in >95% purity with a retention time of 12.0 minutes as a single peak by gamma detection.

### Biological Evaluation of a Somatostatin Receptor Binding Cyclopeptide (FIGS. 31B-31C)

The radiolabeled somatostatin receptor binding molecule was assayed using a standard cellular blocking assay using HT-29 cell line in the presence and absence of 1 mM of the “cold” molecule. The tracer was incubated with the cell for 90 minutes. The solution and cells were separated, washed and analyzed on a gamma counter. The tracer performed consistently to the literature with an observed ˜₈₀% blockage of uptake.

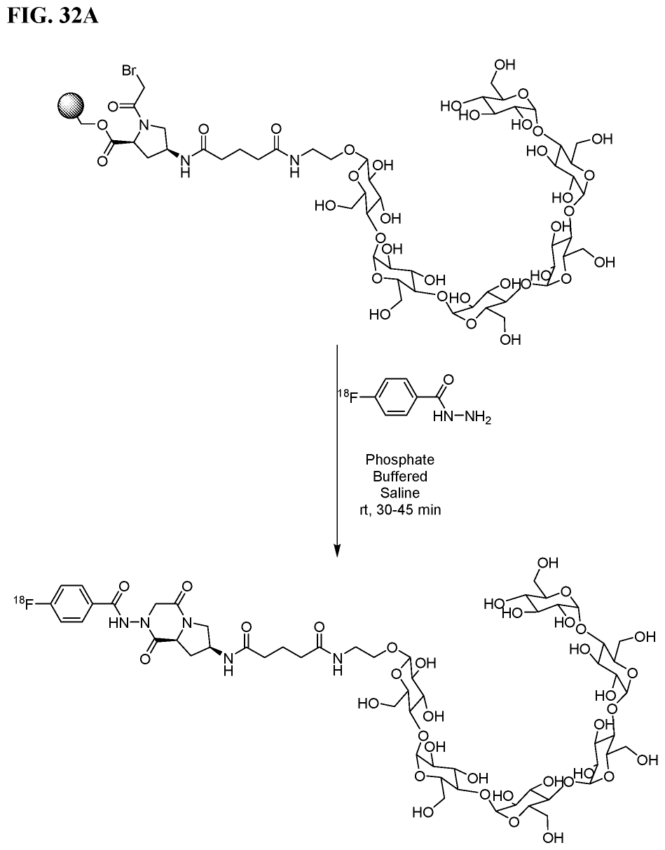

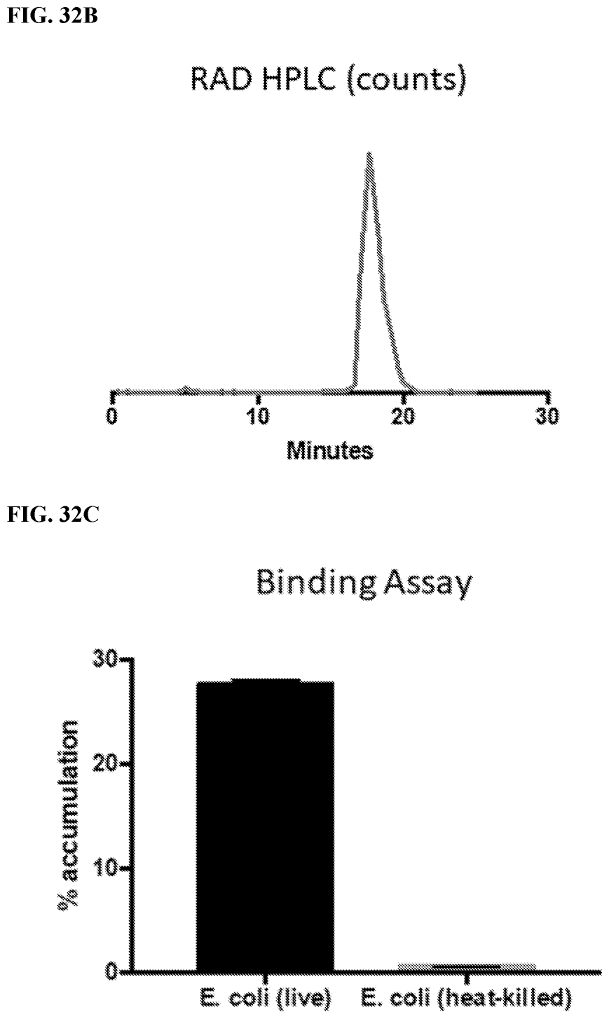

### Radiolabeling of a Bacteria Specific Maltohexaose Molecule (FIG. 32A)

A solution of the 4-[¹⁸F]fluorobenzohydrazide in phosphate buffered saline pH 7.4 was added to the solid supported maltohexaose and agitated for 45 minutes. The solution was collected and analyzed via HPLC to yield the radiolabeled compound in >95% purity with a retention time of 17.7 minutes as a single peak by gamma detection.

### Biological Evaluation of a Bacteria Specific Maltohexaose Molecule (FIGS. 32B-32C)

The radiolabeled bacteria-specific metabolite was assayed using a standard cellular uptake assay using live and heat-killed E. coli pathogen. The tracer was incubated with the cells for 90 minutes. The solution and cells were separated, washed and analyzed on a gamma counter. The tracer performed consistently to the literature with an observed ˜50-fold difference in uptake.

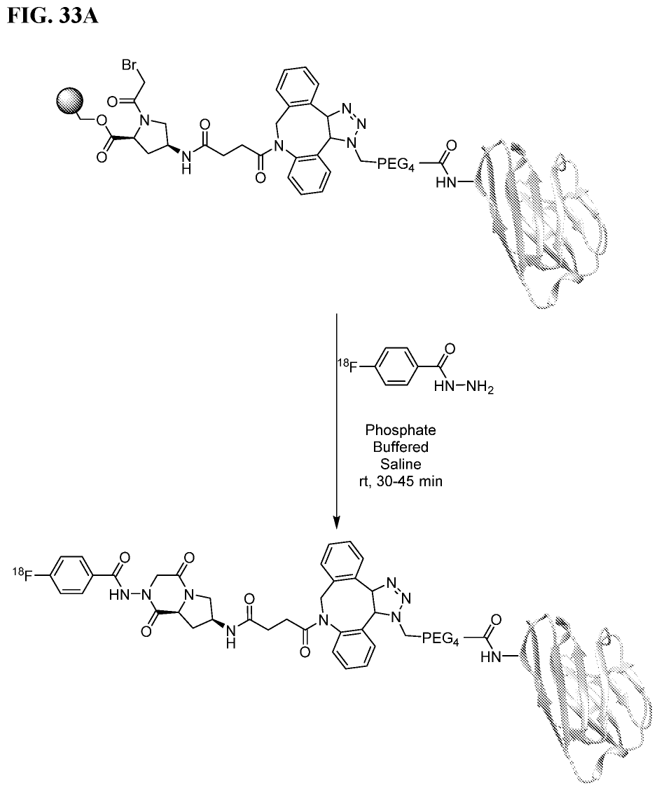

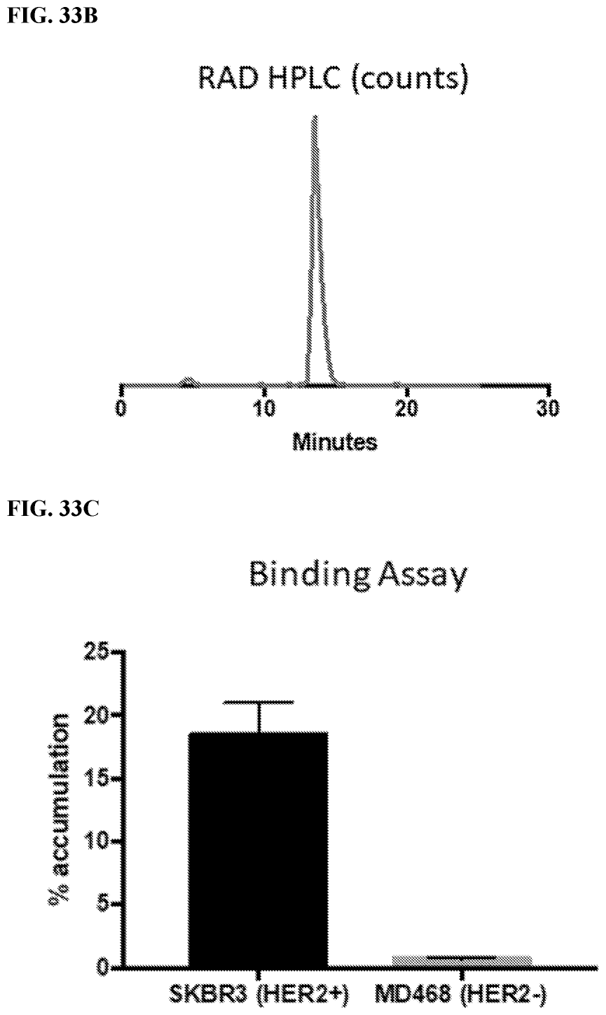

### Radiolabeling of a Her2 Receptor Binding Nanobody (FIG. 33A)

A solution of the 4-[¹⁸F]fluorobenzohydrazide in phosphate buffered saline pH 7.4 was added to the solid supported Her2 binding molecule and agitated for 45 minutes. The solution was collected and analyzed via HPLC to yield the radiolabeled compound in >95% purity with a retention time of 13.5 minutes as a single peak by gamma detection.

### Biological Evaluation of a Her2 Receptor Binding Nanobody (FIGS. 33B-33C)

The radiolabeled Her2 binding molecule was assayed using a standard cellular uptake assay using MD468 cell line (Her2−) and SKBR3 cell line (Her2+). The tracer was incubated with the cell for 90 minutes. The solution and cells were separated, washed and analyzed on a gamma counter. The tracer performed consistently to the literature with an observed ˜20-fold difference in uptake.

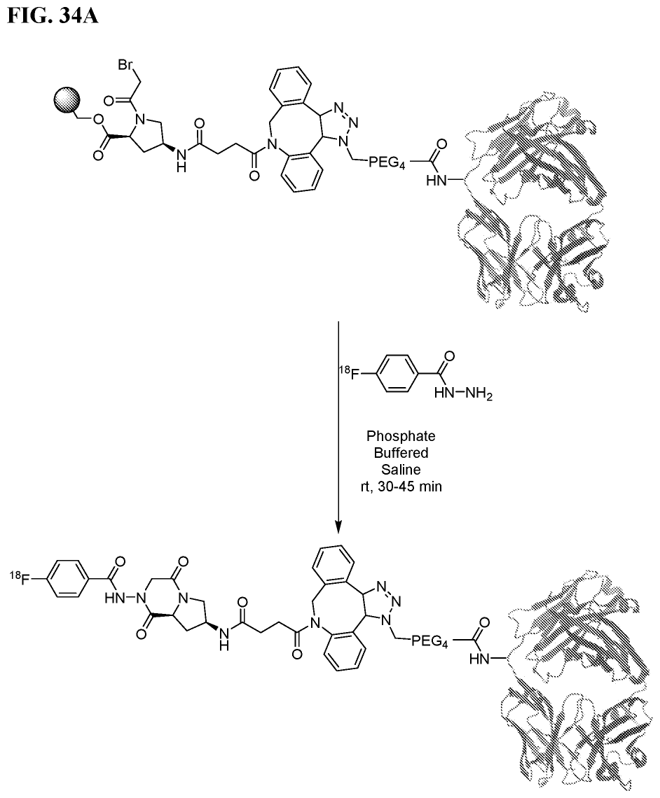

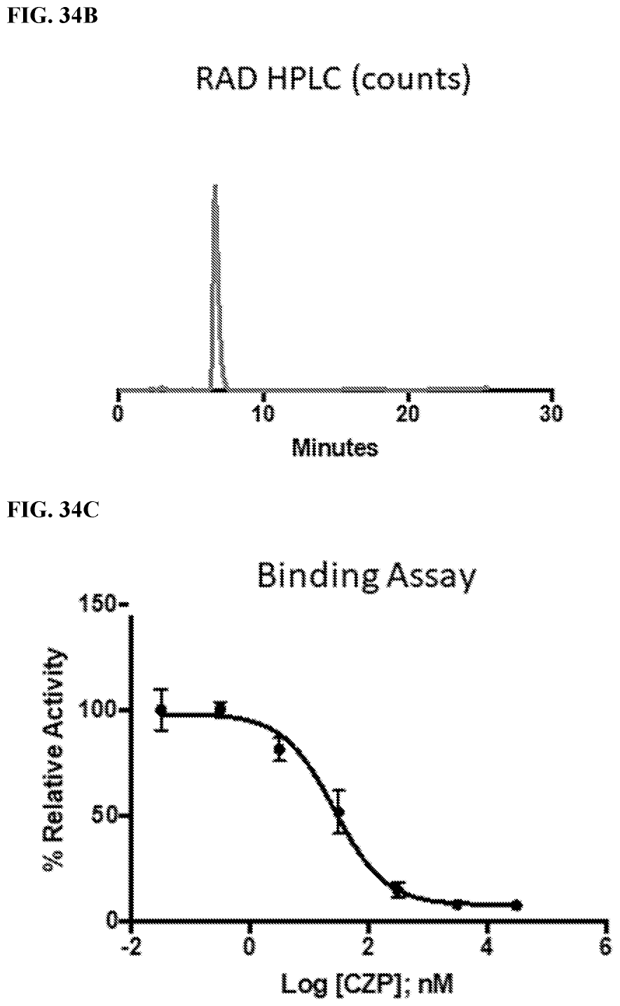

### Radiolabeling of a Tumor Necrosis Factor (TNF) Alpha Binding Antibody Fragment (FIG. 34A)

A solution of the 4-[¹⁸F]fluorobenzohydrazide in phosphate buffered saline pH 7.4 was added to the solid supported TNF alpha binding molecule and agitated for 45 minutes. The solution was collected and analyzed via HPLC to yield the radiolabeled compound in >95% purity with a retention time of 6.6 minutes as a single peak by gamma detection.

### Biological Evaluation of a Tumor Necrosis Factor (TNF) Alpha Binding Antibody Fragment (FIGS. 34B-34C)

The radiolabeled TNF-alpha binding molecule was assayed using a standard ELISA-type binding assay using immobilized TNF-alpha in a well plate in the presence of dilution series of the “cold” antibody fragment. The tracer was incubated with for 90 minutes. The solution and bound tracer were separated, washed and analyzed on a gamma counter. The tracer performed consistently to the literature with an observed IC50 of 26 nM.

What is claimed is:

1. A method of making compound (I^{B}), or a pharmaceutically acceptable alt thereof, said method comprising mixing compound (IV^{B}) or compound (V^{B}) and compound (A) together in a reaction vessel; wherein
compound (I^{B}) has the formula: 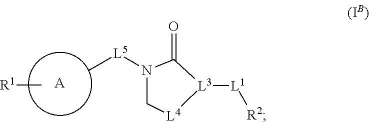
compound (IV^{B}) has the formula: 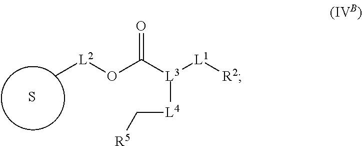
compound (V^{B}) has the formula: 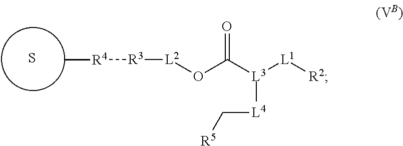
compound (A) has the formula: 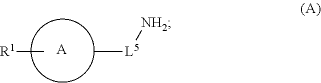
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
---- is a noncovalent or covalent bond; and
S is a solid support.

2. The method of claim 1, wherein
compound (I^{B}) has the formula: 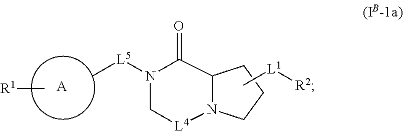
compound (IV^{B}) has the formula: 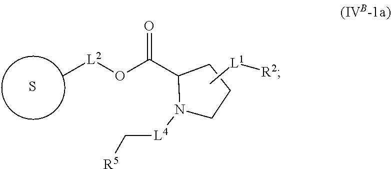
and
compound (V^{B}) has the formula:

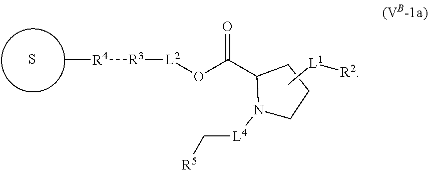

3. The method of claim 1, wherein R⁵ is a halogen,

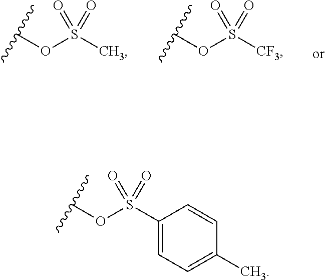 [SMILES: *OS(C)(=O)=O ; *OS(=O)(=O)C(F)(F)F ; *OS(=O)(=O)c1ccc(C)cc1]

4. The method of claim 1, wherein R⁵ is —Br.

5. The method of claim 1, wherein ---- is a noncovalent bond.

6. The method of claim 1, wherein S is a chromatographic material.

7. The method of claim 1, wherein Ring A is a C₃-C₆ cycloalkylene, 3 to 6 membered heterocycloalkylene, or phenylene.

8. The method of claim 1, wherein Ring A is phenylene.

9. The method of claim 1, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted to 6 membered heterocycloalkylene, substituted or unsubstituted phenylene, or substituted or unsubstituted 5 to 6 membered heteroarylene.

10. The method of claim 1, wherein L¹ is —NHC(O)—.

11. The method of claim 1, wherein L² is a substituted or unsubstituted alkylene or substituted or unsubstituted heteroalkylene.

12. The method of claim 1, wherein L² is an unsubstituted C₁-C₄ alkylene.

13. The method of claim 1, wherein L² is an unsubstituted methylene.

14. The method of claim 1, wherein L⁴ is —C(O)—.

15. The method of claim 1, wherein L⁵ is a bond, —NH—, —O—, or —C(O)NH—.

16. The method of claim 1, wherein R¹ is —¹⁸F.

17. The method of one of claims 1 to 16, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

18. The method of one of claims 1 to 16, wherein R² is a monovalent form of a drug or substituted heteroalkyl.

19. The method of one of claims 1 to 16, wherein R² is a monovalent form of a drug.

20. A compound, or a pharmaceutically acceptable salt thereof, having the formula: 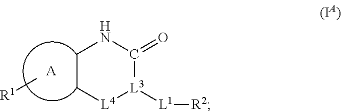
wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4; and
m2 and v2 are each independently 1 or 2.

21. The compound of claim 20, wherein L³ is an unsubstituted heterocycloalkylene.

22. The compound of claim 20, wherein Ring A is a C₃-C₆ cycloalkylene, 3 to 6 membered heterocycloalkylene, or phenylene.

23. The compound of claim 20, wherein Ring A is phenylene.

24. The compound of claim 20, having the formula:

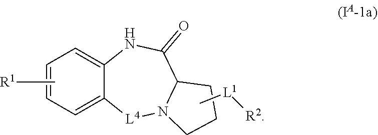

25. The compound of claim 20, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

26. The compound of claim 20, wherein L¹ is —NHC(O)—.

27. The compound of claim 20, wherein L⁴ is —C(O)— or unsubstituted methylene.

28. The compound of claim 20, wherein L⁴ is —C(O)—.

29. The compound of claim 20, wherein R¹ is —¹⁸F.

30. The compound of claim 20, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

31. The compound of claim 20, wherein R² is a monovalent form of a drug or substituted heteroalkyl.

32. The compound of claim 20, wherein R² is a monovalent form of a drug.

33. A pharmaceutical composition comprising a compound of one of claims 20 to 32, or a pharmaceutically acceptable salt thereof, and a pharmaceutically acceptable excipient.

34. A method of detecting a level of a compound in a subject, the method comprising:
(i) administering to the subject a compound of one of claims 20 to 32, or a pharmaceutically acceptable salt thereof, and
(ii) detecting the level of the compound in the subject.

35. The method of claim 34, wherein step (ii) further comprises detecting the level of the compound in the subject using positron emission tomography (PET), wherein R¹ is a PET detectable radioisotope.

36. The method of claim 35, further comprising detecting a physiological location of the compound in the subject using PET.

37. A method of detecting the level of CD44v6 in a subject, the method comprising administering to the subject an effective amount of a compound of one of claims 20 to 32, or a pharmaceutically acceptable salt thereof.

38. The method of claim 37, further comprising detecting the level of the compound using positron emission tomography.

39. A compound, or a pharmaceutically acceptable salt thereof, having the formula: 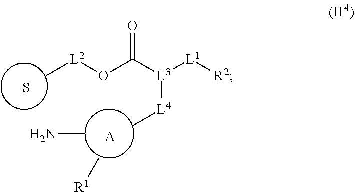
wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
S is a solid support.

40. The compound of claim 39, wherein L³ is an unsubstituted heterocycloalkylene.

41. The compound of claim 39, wherein Ring A is a C₃-C₆ cycloalkylene, 3 to 6 membered heterocycloalkylene, or phenylene.

42. The compound of claim 39, wherein Ring A is phenylene.

43. The compound of claim 39, having the formula:

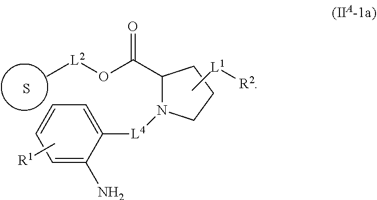

44. A compound, or a pharmaceutically acceptable salt thereof, having the formula: 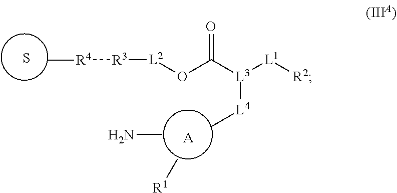
wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
60 R³ and R⁴ are an affinity ligand binding pair;
---- is a noncovalent or covalent bond; and
S is a solid support.

45. The compound of claim 44, wherein L³ is an unsubstituted heterocycloalkylene.

46. The compound of claim 44, wherein Ring A is a C₃-C₆ cycloalkylene, 3 to 6 membered heterocycloalkylene, or phenylene.

47. The compound of claim 44, wherein Ring A is phenylene.

48. The compound of claim 44, having the formula:

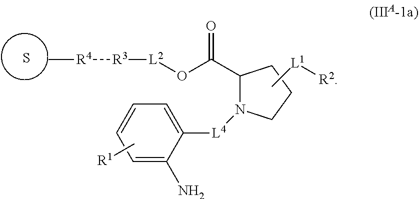

49. The compound of claim 44, wherein ---- is a noncovalent bond.

50. The compound of claim 39, wherein S is a chromatographic material.

51. The compound of claim 39, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

52. The compound of claim 39, wherein L¹ is —NHC(O)—.

53. The compound of claim 39, wherein L² is a substituted or unsubstituted alkylene or substituted or unsubstituted heteroalkylene.

54. The compound of claim 39, wherein L² is an unsubstituted C₁-C₄ alkylene.

55. The compound of claim 39, wherein L² is an unsubstituted methylene.

56. The compound of claim 39, wherein L⁴ is —C(O)— or unsubstituted methylene.

57. The compound of claim 39, wherein L⁴ is —C(O)—.

58. The compound of claim 39, wherein R¹ is —¹⁸F.

59. The compound of one of claims 39 to 58, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

60. The compound of one of claims 39 to 58, wherein R² is a monovalent form of a drug or substituted heteroalkyl.

61. The compound of one of claims 39 to 58, wherein R² is a monovalent form of a drug.

62. A method of making compound (I^{A}), or a pharmaceutically acceptable salt thereof, said method comprising mixing compound (II^{A}) or compound (III^{A}) in a reaction vessel; wherein
compound (I^{A}) has the formula: 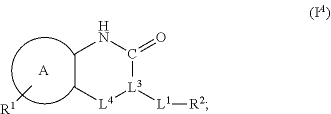
compound (II^{A}) has the formula: 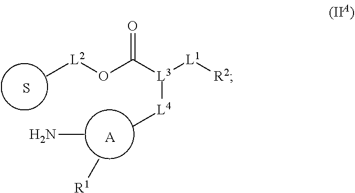
compound (III^{A}) has the formula: 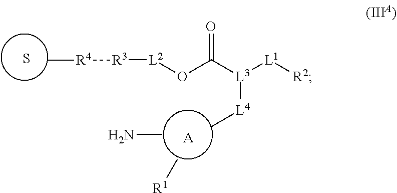
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C} and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

63. The method of claim 62, further comprising mixing compound (II^{A}) or compound (III^{A}) in a basic solution.

64. The method of claim 63, wherein the basic solution has a pH from 7.1 to 8.

65. The method of claim 63, wherein the basic solution has a pH of 7.4.

66. The method of claim 62, wherein L³ is an unsubstituted heterocycloalkylene.

67. The method of claim 62, wherein Ring A is a C₃-C₆ cycloalkylene, 3 to membered heterocycloalkylene, or phenylene.

68. The method of claim 62, wherein Ring A is phenylene.

69. The method of claim 62, wherein
compound (I^{A}) has the formula: 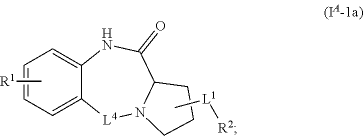
compound (II^{A}) has the formula: 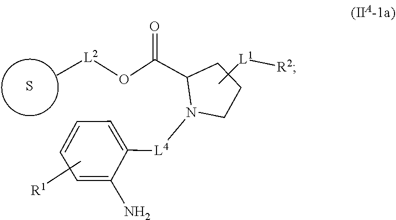
and
compound (III^{A}) has the formula:

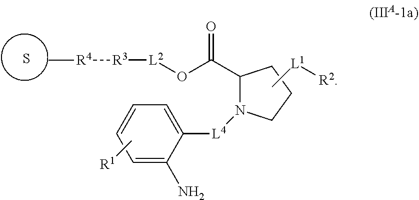

70. The method of claim 62, wherein ---- is a noncovalent bond.

71. The method of claim 62, wherein S is a chromatographic material.

72. The method of claim 62, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted to 6 membered heterocycloalkylene, substituted or unsubstituted phenylene, or substituted or unsubstituted 5 to 6 membered heteroarylene.

73. The method of claim 62, wherein L¹ is —NHC(O)—.

74. The method of claim 62, wherein L² is a substituted or unsubstituted alkylene or substituted or unsubstituted heteroalkylene.

75. The method of claim 62, wherein L² is an unsubstituted C₁-C₄ alkylene.

76. The method of claim 62, wherein L² is an unsubstituted methylene.

77. The method of claim 62, wherein L⁴ is —C(O)— or unsubstituted methylene.

78. The method of claim 62, wherein L⁴ is —C(O)—.

79. The method of claim 62, wherein R¹ is —¹⁸F.

80. The method of one of claims 62 to 79, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

81. The method of one of claims 62 to 79, wherein R² is a monovalent form of a drug or substituted heteroalkyl.

82. The method of one of claims 62 to 79, wherein R² is a monovalent form of a drug.

83. A compound, or a pharmaceutically acceptable salt thereof, having the formula: 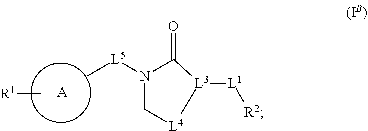
wherein
Ring A is a C₃-C₆ cycloalkylene, 3 to 6 membered heterocycloalkylene, phenylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4; and
m2 and v2 are each independently 1 or 2.

84. The compound of claim 83, wherein L³ is an unsubstituted heterocycloalkylene.

85. The compound of claim 83, having the formula:

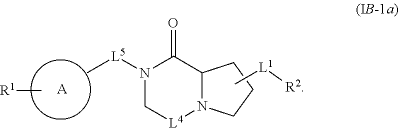

86. The compound of claim 83, wherein Ring A is a C₃-C₆ cycloalkylene, 3 to 6 membered heterocycloalkylene, or phenylene.

87. The compound of claim 83, wherein Ring A is phenylene.

88. The compound of claim 83, wherein L⁴ is —C(O)—.

89. The compound of claim 83, wherein L⁵ is a bond, —NH—, —O—, or —C(O)NH—.

90. The compound of claim 83, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

91. The compound of claim 83, wherein L¹ is —NHC(O)—.

92. The compound of claim 83, wherein R¹ is —¹⁸F.

93. The compound of claim 83, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

94. The compound of claim 83, wherein R² is a monovalent form of a drug or substituted heteroalkyl.

95. The compound of claim 83, wherein R² is a monovalent form of a drug.

96. A pharmaceutical composition comprising a compound of one of claims 83 to 95, or a pharmaceutically acceptable salt thereof, and a pharmaceutically acceptable excipient.

97. A method of detecting a level of a compound in a subject, the method comprising:
(i) administering to the subject a compound of one of claims 83 to 95, or a pharmaceutically acceptable salt thereof, and
(ii) detecting the level of the compound in the subject.

98. The method of claim 97, wherein step (ii) further comprises detecting the level of the compound in the subject using positron emission tomography (PET), wherein R¹ is a PET detectable radioisotope.

99. The method of claim 98, further comprising detecting a physiological location of the compound in the subject using PET.

100. A method of detecting the level of CD44v6 in a subject, the method comprising administering to the subject an effective amount of a compound of one of claims 83 to 95, or a pharmaceutically acceptable salt thereof.

101. The method of claim 100, further comprising detecting the level of the compound using positron emission tomography.

102. A compound, or a pharmaceutically acceptable salt thereof, having the formula: 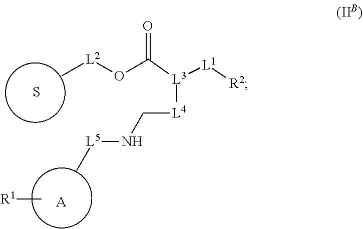
wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
S is a solid support.

103. The compound of claim 102, having the formula:

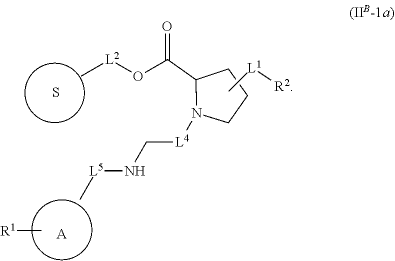

104. A compound, or a pharmaceutically acceptable salt thereof, having the formula: 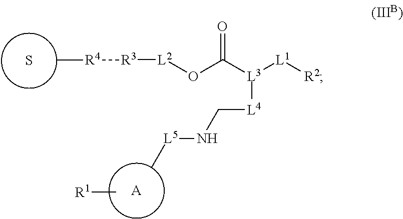
wherein
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C} and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

105. The compound of claim 104, having the formula:

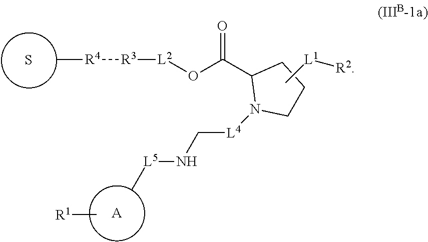

106. The compound of claim 104, wherein ---- is a noncovalent bond.

107. The compound of claim 102, wherein Ring A is a C₃-C₆ cycloalkylene, 3 to 6 membered heterocycloalkylene, or phenylene.

108. The compound of claim 102, wherein Ring A is phenylene.

109. The compound of claim 102, wherein R¹ is —¹⁸F.

110. A compound, or a pharmaceutically acceptable salt thereof, having the formula: 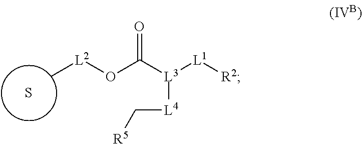
wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R⁵ is a leaving group; and
S is a solid support.

111. The compound of claim 110, having the formula:

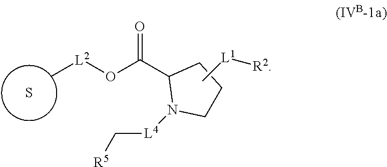

112. A compound, or a pharmaceutically acceptable salt thereof, having the formula: 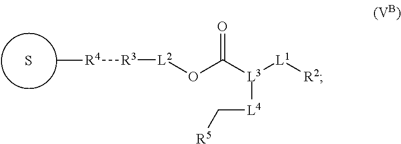
wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
---- is a noncovalent or covalent bond; and
S is a solid support.

113. The compound of claim 112, having the formula:

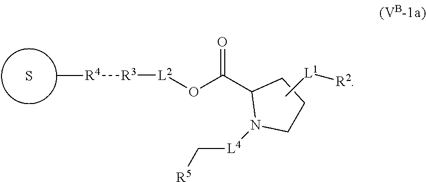

114. The compound of claim 112, wherein ---- is a noncovalent bond.

115. The compound of claim 110, wherein R⁵ is a halogen,

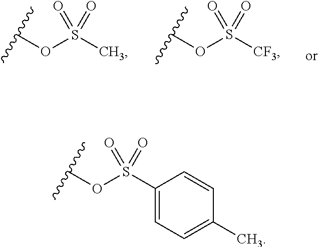 [SMILES: *OS(C)(=O)=O ; *OS(=O)(=O)C(F)(F)F ; *OS(=O)(=O)c1ccc(C)cc1]

116. The compound of claim 110, wherein R⁵ is —Br.

117. The compound of claim 102, wherein S is a chromatographic material.

118. The compound of claim 102, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

119. The compound of claim 102, wherein L¹ is —NHC(O)—.

120. The compound of claim 102, wherein L² is a substituted or unsubstituted alkylene or substituted or unsubstituted heteroalkylene.

121. The compound of claim 102, wherein L² is an unsubstituted C₁-C₄ alkylene.

122. The compound of claim 102, wherein L² is an unsubstituted methylene.

123. The compound of claim 102, wherein L⁴ is —C(O)—.

124. The compound of one of claims 102 to 123, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

125. The compound of one of claims 102 to 123, wherein R² is a monovalent form of a drug or substituted heteroalkyl.

126. The compound of one of claims 102 to 123, wherein R² is a monovalent form of a drug.

127. A method of making compound (I^{B}), or a pharmaceutically acceptable salt thereof, said method comprising mixing compound (II^{B}) or compound (III^{B}) in a reaction vessel; wherein
compound (I^{B}) has the formula: 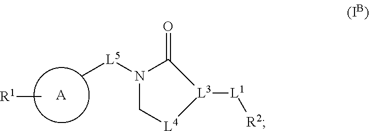
compound (II^{B}) has the formula: 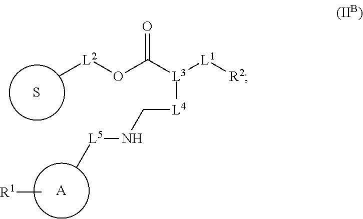
compound (III^{B}) has the formula: 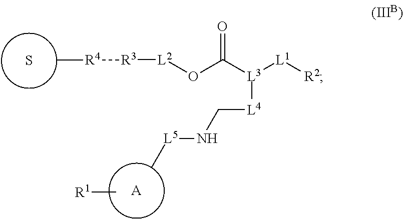
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L³ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C} and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
---- is a noncovalent or covalent bond; and
S is a solid support.

128. The method of claim 127, wherein
compound (I^{B}) has the formula: 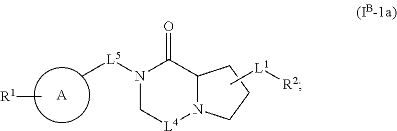
compound (II^{B}) has the formula: 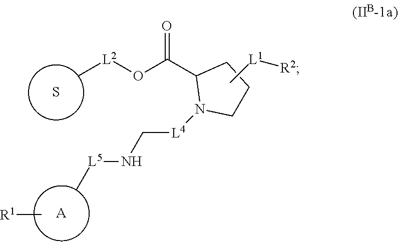
and
compound (III^{B}) has the formula:

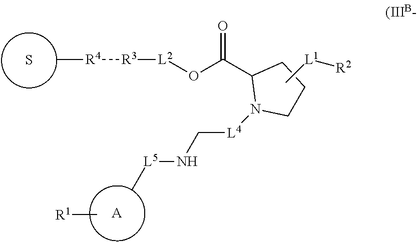

129. The method of claim 127, further comprising mixing compound (II^{B}) or compound (III^{B}) in a basic solution.

130. The method of claim 129, wherein the basic solution has a pH from 7.1 to 8.

131. The method of claim 129, wherein the basic solution has a pH of 7.4.

132. The method of claim 127, wherein ---- is a noncovalent bond.

133. The method of claim 127, wherein S is a chromatographic material.

134. The method of claim 127, wherein Ring A is a C₃-C₆ cycloalkylene, 3 to 6 membered heterocycloalkylene, or phenylene.

135. The method of claim 127, wherein Ring A is phenylene.

136. The method of claim 127, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

137. The method of claim 127, wherein L¹ is —NHC(O)—.

138. The method of claim 127, wherein L² is a substituted or unsubstituted alkylene or substituted or unsubstituted heteroalkylene.

139. The method of claim 127, wherein L² is an unsubstituted C₁-C₄ alkylene.

140. The method of claim 127, wherein L² is an unsubstituted methylene.

141. The method of claim 127, wherein L⁴ is —C(O)—.

142. The method of claim 127, wherein L⁵ is a bond, —NH—, —O—, or —C(O)NH—.

143. The method of claim 127, wherein R¹ is —¹⁸F.

144. The method of one of claims 127 to 143, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

145. The method of one of claims 127 to 143, wherein R² is a monovalent form of a drug or substituted heteroalkyl.

146. The method of one of claims 127 to 143, wherein R² is a monovalent form of a drug.

147. A compound, or a pharmaceutically acceptable salt thereof, having the formula: 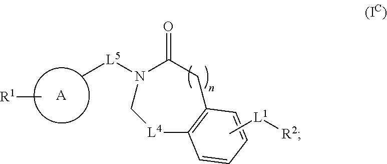
wherein
Ring A is a C₃-C₆ cycloalkylene, 3 to 6 membered heterocycloalkylene, phenylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2; and
n is 0 or 1.

148. The compound of claim 147, wherein Ring A is a C₃-C₆ cycloalkylene, 3 to 6 membered heterocycloalkylene, or phenylene.

149. The compound of claim 147, wherein Ring A is phenylene.

150. The compound of claim 147, wherein L⁴ is a bond.

151. The compound of claim 147, wherein L⁵ is a bond.

152. The compound of claim 147, wherein n is 0.

153. The compound of claim 147, wherein n is 1.

154. The compound of claim 147, having the formula:

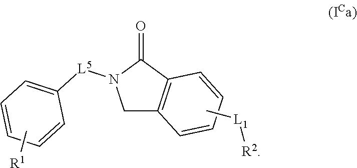

155. The compound of claim 147, having the formula:

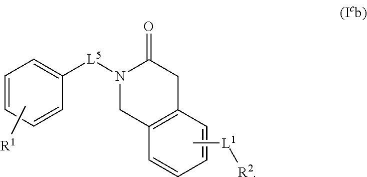

156. The compound of claim 147, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

157. The compound of claim 147, wherein L¹ is —NHC(O)—.

158. The compound of claim 147, wherein R¹ is —¹⁸F.

159. The compound of claim 147, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

160. The compound of claim 147, wherein R² is a monovalent form of a drug or substituted heteroalkyl.

161. The compound of claim 147, wherein R² is a monovalent form of a drug.

162. A pharmaceutical composition comprising a compound of one of claims 147 to 161, or a pharmaceutically acceptable salt thereof, and a pharmaceutically acceptable excipient.

163. A method of detecting the level of a compound in a subject, the method comprising:
(i) administering to the subject a compound of one of claims 147 to 161, or a pharmaceutically acceptable salt thereof, and
(ii) detecting the level of the compound in the subject.

164. The method of claim 163, wherein step (ii) further comprises detecting the level of the compound in the subject using positron emission tomography (PET), wherein R¹ is a PET detectable radioisotope.

165. The method of claim 164, further comprising detecting a physiological location of the compound in the subject using PET.

166. A method of detecting the level of CD44v6 in a subject, the method comprising administering to the subject an effective amount of a compound of one of claims 147 to 161, or a pharmaceutically acceptable salt thereof.

167. The method of claim 166, further comprising detecting the level of the compound using positron emission tomography.

168. A compound, or a pharmaceutically acceptable salt thereof, having the formula: 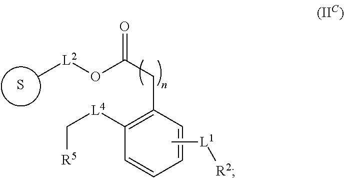
wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R⁵ is a leaving group;
n is 0 or 1; and
S is a solid support.

169. A compound, or a pharmaceutically acceptable salt thereof, having the formula: 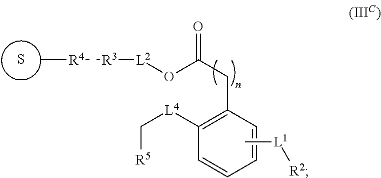
wherein
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰-, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C}, and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
n is 0 or 1;
---- is a noncovalent or covalent bond; and
S is a solid support.

170. The compound of claim 169, wherein ---- is a noncovalent bond.

171. The compound of claim 168, wherein S is a chromatographic material.

172. The compound of claim 168, wherein n is 0.

173. The compound of claim 168, wherein n is 1.

174. The compound of claim 168, wherein R⁵ is a halogen,

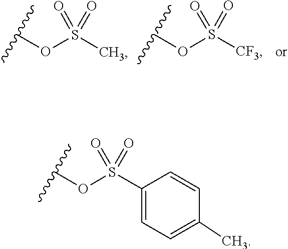 [SMILES: *OS(C)(=O)=O ; *OS(=O)(=O)C(F)(F)F ; *OS(=O)(=O)c1ccc(C)cc1]

175. The compound of claim 168, wherein R⁵ is —Br.

176. The compound of claim 168, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

177. The compound of claim 168, wherein L¹ is —NHC(O)—.

178. The compound of claim 168, wherein L² is a substituted or unsubstituted alkylene or substituted or unsubstituted heteroalkylene.

179. The compound of claim 168, wherein L² is an unsubstituted C₁-C₄ alkylene.

180. The compound of claim 168, wherein L² is an unsubstituted methylene.

181. The compound of claim 168, wherein L⁴ is a bond.

182. The compound of one of claims 168 to 181, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

183. The compound of one of claims 168 to 181, wherein R² is a monovalent form of a drug or substituted heteroalkyl.

184. The compound of one of claims 168 to 181, wherein R² is a monovalent form of a drug.

185. A method of making compound (I^{C}), or a pharmaceutically acceptable salt thereof, said method comprising mixing compound (II^{C}) or compound (III^{C}) and compound (A) together in a reaction vessel; wherein
compound (I^{C}) has the formula: 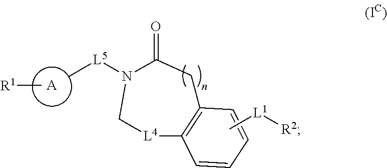
compound (II^{C}) has the formula: 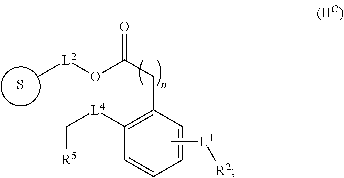
compound (III^{C}) has the formula: 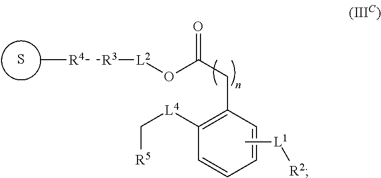
compound (A) has the formula: 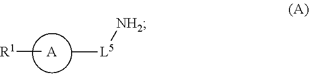
Ring A is a C₃-C₆ cycloalkylene, 3 to 6 membered heterocycloalkylene, C₅-C₆ arylene, or 5 to 6 membered heteroarylene;
L¹ is a bond, —N(R¹⁰)—, —S—, —S(O)₂—, —O—, —C(O)—, —C(O)O—, —OC(O)—, —N(R¹⁰)C(O)—, —C(O)N(R¹⁰)—, —NR¹⁰C(O)NR¹⁰—, —NR¹⁰C(NH)NR¹⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R²⁰)—, —S—, —S(O)₂—, —O—, —C(O)—, —C(O)O—, —OC(O)—, —N(R²⁰)C(O)—, —C(O)N(R²⁰)—, —NR²⁰C(O)NR²⁰—, —NR²⁰C(NH)NR²⁰—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R²⁰ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond, —C(O)—, substituted or unsubstituted C₁-C₆ alkylene, or substituted or unsubstituted 2 to 6 membered heteroalkylene;
L⁵ is a bond, —NH—, —O—, —S(O)₂—, —C(O)NH—, or substituted or unsubstituted C₁-C₆ alkylene;
R¹ is a radioisotope;
R² is a monovalent form of a drug, hydrogen, halogen, —CX²₃, —CHX²₂, —CH₂X², —OCX²₃, —OCHX²₂, —OCH₂X², —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR²C, —NR^{2A}OR²C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a monovalent form of a biomolecule;
R^{2A}, R^{2B}, R^{2C} and R^{2D} are each independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl, or substituted or unsubstituted heteroaryl;
X² is —F, —Cl, —Br, or —I;
n2 is an integer from 0 to 4;
m2 and v2 are each independently 1 or 2;
R³ and R⁴ are an affinity ligand binding pair;
R⁵ is a leaving group;
n is 0 or 1;
---- is a noncovalent or covalent bond; and
S is a solid support.

186. The method of claim 185, wherein ---- is a noncovalent bond.

187. The method of claim 185, wherein S is a chromatographic material.

188. The method of claim 185, wherein Ring A is a C₃-C₆ cycloalkylene, 3 to 6 membered heterocycloalkylene, or phenylene.

189. The method of claim 185, wherein Ring A is phenylene.

190. The method of claim 185, wherein L⁴ is a bond.

191. The method of claim 185, wherein L⁵ is a bond.

192. The method of claim 185, wherein R⁵ is a halogen,

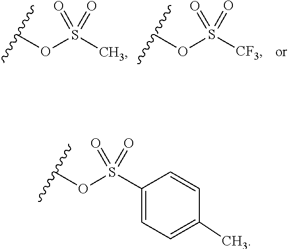 [SMILES: *OS(C)(=O)=O ; *OS(=O)(=O)C(F)(F)F ; *OS(=O)(=O)c1ccc(C)cc1]

193. The method of claim 185, wherein R⁵ is —Br.

194. The method of claim 185, wherein n is 0.

195. The method of claim 185, wherein n is 1.

196. The method of claim 185, wherein
compound (I^{C}) has the formula: 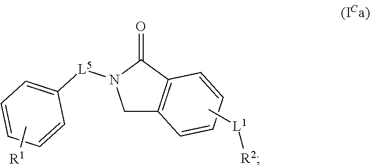
compound (II^{C}) has the formula: 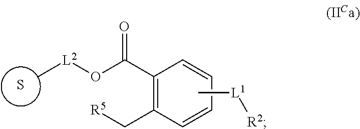
and
compound (III^{C}) has the formula:

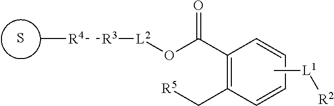

197. The method of claim 185, wherein
compound (I^{C}) has the formula: 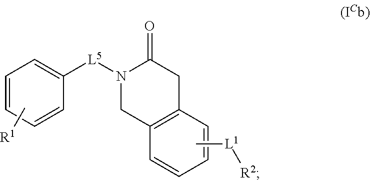
compound (II^{C}) has the formula: 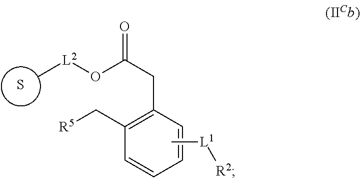
and
compound (III^{C}) has the formula:

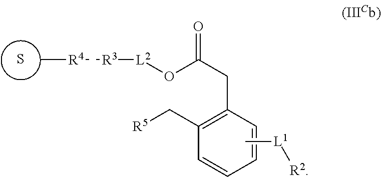

198. The method of claim 185, wherein L¹ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —C(S)—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

199. The method of claim 185, wherein L¹ is —NHC(O)—.

200. The method of claim 185, wherein L² is a substituted or unsubstituted alkylene or substituted or unsubstituted heteroalkylene.

201. The method of claim 185, wherein L² is an unsubstituted C₁-C₄ alkylene.

202. The method of claim 185, wherein L² is an unsubstituted methylene.

203. The method of claim 185, wherein R¹ is —¹⁸F.

204. The method of one of claims 185 to 203, wherein R² is a monovalent form of a drug, hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

205. The method of one of claims 185 to 203, wherein R² is a monovalent form of a drug or substituted heteroalkyl.

206. The method of one of claims 185 to 203, wherein R² is a monovalent form of a drug.


SEQUENCE LISTING (5 sequences)

SEQ ID NO: 1 - protein, 14 aa, Artificial Sequence
  Synthetic polypeptide
GSGSGSGALAYADA

SEQ ID NO: 2 - protein, 15 aa, Artificial Sequence
  Synthetic polypeptide
DATFNWVFPVSVTFP

SEQ ID NO: 3 - protein, 15 aa, Artificial Sequence
  Synthetic polypeptide
RAGAYYVSSYRPGAW

SEQ ID NO: 4 - protein, 7 aa, Artificial Sequence
  Synthetic polypeptide
LPRDYAS

SEQ ID NO: 5 - protein, 7 aa, Artificial Sequence
  Synthetic polypeptide
DYGKNSW